(19) United States
(12) Patent Application Publication
(10) Pub. No.: US 2024/0307175 A1
(43) Pub. Date: Sep. 19, 2024

(54) PROSTHETIC VALVES FOR IMPLANTATION

(71) Applicant: EDWARDS LIFESCIENCES CORPORATION, Irvine, CA (US)

(72) Inventors: Alexander H. Cooper, Costa Mesa, CA (US); Juliet Laura Schwartz, Tustin, CA (US); Hannah Marie Glas, Irvine, CA (US); Matthew Michael Becerra, Lake Forest, CA (US); David Robert Landon, Huntington Beach, CA (US); Siddharth Vad, Irvine, CA (US); Taylor Jacob Scheinblum, Newport Beach, CA (US); Matthew A. Peterson, Costa Mesa, CA (US); Wei Gan, Irvine, CA (US)

(21) Appl. No.: 18/673,188

(22) Filed: May 23, 2024

Related U.S. Application Data
(63) Continuation of application No. PCT/US2022/050161, filed on Nov. 16, 2022.
(60) Provisional application No. 63/282,662, filed on Nov. 23, 2021.

(51) Int. Cl. A61F 2/24

(52) U.S. Cl. CPC A61F 2/2418; A61F 2/2433; A61F 2220/0016; A61F 2220/0075; A61F 2230/0069; A61F 2250/006; A61F 2250/0068

(57) ABSTRACT

Apparatuses, systems, and methods for prosthetic valves. Examples of prosthetic valves may be configured to anchor to an interior surface of a heart valve. Such prosthetic valves, in examples, may include a single frame that supports a plurality of prosthetic valve leaflets. In examples, multiple frames may be utilized to anchor to a heart valve and one or more of the frames may support a plurality of prosthetic valve leaflets.

### CROSS-REFERENCE TO RELATED APPLICATIONS

This application is a continuation of International Application No. PCT/US2022/050161, filed Nov. 16, 2022, which designates the United States and was published in English by the International Bureau on Jun. 1, 2023 as WO2023/096804, which claims the benefit of U.S. Provisional Application No. 63/282,662, filed Nov. 23, 2021, the contents of which are hereby incorporated by reference.

### BACKGROUND

### Field

Certain examples disclosed herein relate generally to implants, including prosthetic valves for implantation.

### Background

Human heart valves, which include the aortic, pulmonary, mitral, and tricuspid valves, function essentially as one-way valves operating in synchronization with the pumping heart. The valves allow blood to flow downstream, but block blood from flowing upstream. Diseased heart valves exhibit impairments such as narrowing of the valve or regurgitation, which inhibit the valves' ability to control blood flow. Such impairments reduce the heart's blood-pumping efficiency and can be a debilitating and life-threatening condition. For example, valve insufficiency can lead to conditions such as heart hypertrophy and dilation of the ventricle. Thus, extensive efforts have been made to develop methods and apparatuses to repair or replace impaired heart valves.

Prostheses exist to correct problems associated with impaired heart valves. For example, mechanical and tissue-based heart valve prostheses can be used to replace impaired native heart valves. More recently, substantial effort has been dedicated to developing replacement heart valves, particularly tissue-based replacement heart valves that can be delivered with less trauma to the patient than through open heart surgery. Replacement valves are being designed to be delivered through minimally invasive procedures and even percutaneous procedures. Such replacement valves often include a tissue-based valve body that is connected to an expandable frame that is then delivered to the native valve's annulus.

These replacement valves may be deployed with anchors that are configured to anchor to a portion of a heart valve. Such anchoring processes, however, may be difficult to visualize and it may be difficult to determine if the anchoring has occurred properly. Improved anchoring methods may often result in larger, more bulky valves, that may require larger entry points into the body and larger delivery systems for the replacement valves.

### SUMMARY

Examples of prosthetic valves disclosed herein may be configured to anchor to an interior surface of a heart valve. Such prosthetic valves, in examples, may include a single frame that supports a plurality of prosthetic valve leaflets. In examples, multiple frames may be utilized to anchor to a heart valve and one or more of the frames may support a plurality of prosthetic valve leaflets.

Examples herein may be directed to improved assembly and preparation methods for implants such as prosthetic valves.

Examples as disclosed herein may include a prosthetic valve for implantation within a heart valve. The prosthetic valve may comprise a plurality of prosthetic valve leaflets, a valve frame configured to support the plurality of prosthetic valve leaflets, and a plurality of elongate anchor arms each having a first end portion coupled to the valve frame and each configured to extend radially outward from the valve frame to a second end portion that is configured to anchor to an interior surface of the heart valve.

Examples as disclosed herein may include a method. The method may comprise deploying a prosthetic valve within a heart valve. The prosthetic valve may include a plurality of prosthetic valve leaflets, a valve frame configured to support the plurality of prosthetic valve leaflets, and a plurality of elongate anchor arms each having a first end portion coupled to the valve frame and each configured to extend radially outward from the valve frame to a second end portion that is configured to anchor to an interior surface of the heart valve.

Examples as disclosed herein may include a prosthetic valve for implantation within a heart valve. The prosthetic valve may comprise a plurality of prosthetic valve leaflets, a valve frame configured to support the plurality of prosthetic valve leaflets, a plurality of anchors each configured to extend radially outward from the valve frame and be positioned radially outward of the heart valve to anchor to the heart valve, and a plurality of sealing bodies configured to extend radially outward from the valve frame, each positioned between two of the plurality of anchors and configured to be positioned radially inward of the heart valve to seal fluid flow.

Examples as disclosed herein may include a method. The method may comprise deploying a prosthetic valve within a heart valve. The prosthetic valve may include a plurality of prosthetic valve leaflets, a valve frame configured to support the plurality of prosthetic valve leaflets, a plurality of anchors each configured to extend radially outward from the valve frame and be positioned radially outward of the heart valve to anchor to the heart valve, and a plurality of sealing bodies configured to extend radially outward from the valve frame, each positioned between two of the plurality of anchors and configured to be positioned radially inward of the heart valve to seal fluid flow.

Examples as disclosed herein may include a prosthetic valve for implantation within a heart valve. The prosthetic valve may comprise a plurality of prosthetic valve leaflets, and an anchoring body coupled to the plurality of prosthetic valve leaflets and configured to anchor to the heart valve. The anchoring body may include a proximal frame and a distal frame and configured to form an annular recess between the proximal frame and the distal frame for receiving a portion of the heart valve, the proximal frame may extend radially outward from a proximal end portion to form a first shoulder positioned proximal of the annular recess, and the distal frame may extend radially outward from a distal end portion to form a second shoulder positioned distal of the annular recess.

Examples as disclosed herein may include a method. The method may comprise deploying a prosthetic valve within a heart valve. The prosthetic valve may include a plurality of prosthetic valve leaflets, and an anchoring body coupled to the plurality of prosthetic valve leaflets and configured to anchor to the heart valve. The anchoring body may include a proximal frame and a distal frame and configured to form an annular recess between the proximal frame and the distal frame for receiving a portion of the heart valve, the proximal frame extending radially outward from a proximal end portion to form a first shoulder positioned proximal of the annular recess, and the distal frame extending radially outward from a distal end portion to form a second shoulder positioned distal of the annular recess.

Examples as disclosed herein may include a prosthetic valve for implantation within a heart valve. The prosthetic valve may comprise a plurality of prosthetic valve leaflets, an inner frame supporting the plurality of prosthetic valve leaflets and including a plurality of anchors for anchoring the prosthetic valve to the heart valve, an outer frame configured to be positioned radially outward of the inner frame, and one or more tethers coupling the outer frame to the inner frame and configured to impede movement of the outer frame radially inward to reduce interlacing of the outer frame with the inner frame.

Examples as disclosed herein may include a method. The method may comprise deploying a prosthetic valve within a heart valve. The prosthetic valve may include a plurality of prosthetic valve leaflets, an inner frame supporting the plurality of prosthetic valve leaflets and including a plurality of anchors for anchoring the prosthetic valve to the heart valve, an outer frame configured to be positioned radially outward of the inner frame, and one or more tethers coupling the outer frame to the inner frame and configured to impede movement of the outer frame radially inward to reduce interlacing of the outer frame with the inner frame.

Examples as disclosed herein may include a method. The method may comprise compressing a prosthetic valve radially inward to crimp the prosthetic valve. The prosthetic valve may include a plurality of prosthetic valve leaflets, an inner frame supporting the plurality of prosthetic valve leaflets and including a plurality of anchors for anchoring the prosthetic valve to the heart valve, an outer frame configured to be positioned radially outward of the inner frame, and one or more tethers coupling the outer frame to the inner frame and configured to impede movement of the outer frame radially inward to reduce interlacing of the outer frame with the inner frame during the crimping of the prosthetic valve.

Examples as disclosed herein may include a prosthetic valve for implantation within a heart valve. The prosthetic valve may comprise a plurality of prosthetic valve leaflets, an inner frame supporting the plurality of prosthetic valve leaflets, and an outer frame configured to be positioned radially outward of the inner frame and configured to be assembled to the inner frame within a patient's body.

Examples as disclosed herein may include a method. The method may comprise deploying a prosthetic valve within a heart valve. The prosthetic valve may include a plurality of prosthetic valve leaflets, an inner frame supporting the plurality of prosthetic valve leaflets, and an outer frame configured to be positioned radially outward of the inner frame and configured to be assembled to the inner frame within a patient's body.

Examples as disclosed herein may include a prosthetic valve for implantation within a heart valve. The prosthetic valve may comprise a plurality of prosthetic valve leaflets, an inner valve body supporting the plurality of prosthetic valve leaflets and configured to be expanded with an inflatable body, and a self-expanding outer valve body configured to extend radially outward from the inner valve body.

Examples as disclosed herein may include a method. The method may comprise deploying a prosthetic valve within a heart valve. The prosthetic valve may include a plurality of prosthetic valve leaflets, an inner valve body supporting the plurality of prosthetic valve leaflets and configured to be expanded with an inflatable body, and a self-expanding outer valve body configured to extend radially outward from the inner valve body.

Examples as disclosed herein may include a system. The system may include a prosthetic valve configured to be deployed to a heart valve of a patient's heart, at least a portion of the prosthetic valve having a dimension that has been reduced with electropolishing.

Examples as disclosed herein may include a method. The method may include electropolishing at least a portion of a body for use with a prosthetic valve, the electropolishing reducing a dimension of at least the portion of the body.

Examples as disclosed herein may include a system. The system may include a prosthetic valve configured to be deployed to a heart valve of a patient's heart, at least a portion of the prosthetic valve being configured to include one or more drugs for interaction with the patient's body.

### BRIEF DESCRIPTION OF THE DRAWINGS

Features and advantages of the systems, apparatuses, and methods as disclosed herein will become appreciated as the same become better understood with reference to the specification, claims, and appended drawings wherein:

FIG. 1 illustrates a side view of a frame of a prosthetic valve according to examples of the present disclosure.

FIG. 2 illustrates a perspective view of the frame shown in FIG. 1.

FIG. 3 illustrates a schematic cross sectional view of a prosthetic valve utilizing the frame shown in FIG. 1.

FIG. 4 illustrates a plan view of the frame of the prosthetic valve shown in FIG. 1.

FIG. 5 illustrates a schematic view of a delivery apparatus approaching a left ventricle of a patient's heart.

FIG. 6 illustrates a side view of a distal end of a delivery apparatus.

FIG. 7 illustrates a schematic cross sectional view of the prosthetic valve shown in FIG. 3 approaching a native heart valve.

FIG. 8 illustrates a schematic cross sectional view of the prosthetic valve shown in FIG. 3 being deployed.

FIG. 9 illustrates a schematic cross sectional view of the prosthetic valve shown in FIG. 3 being deployed.

FIG. 10 illustrates a schematic cross sectional view of the prosthetic valve shown in FIG. 3 being deployed.

FIG. 11 illustrates a schematic cross sectional view of the prosthetic valve shown in FIG. 3 deployed.

FIGS. 12A-F illustrate a plan view of anchor arms according to examples herein.

FIG. 13 illustrates a plan view of a frame of a prosthetic valve according to examples of the present disclosure.

FIG. 14 illustrates a perspective view of the frame shown in FIG. 13.

FIG. 15 illustrates a schematic cross sectional view of a prosthetic valve utilizing the frame shown in FIG. 13.

FIG. 16 illustrates a schematic cross sectional view of a prosthetic valve utilizing the frame shown in FIG. 13 deployed to a heart valve.

FIG. 17 illustrates a schematic cross sectional view of a prosthetic valve according to examples of the present disclosure.

FIG. 18 illustrates a schematic cross sectional view of the prosthetic valve shown in FIG. 17 approaching a native heart valve.

FIG. 19 illustrates a schematic cross sectional view of the prosthetic valve shown in FIG. 17 being deployed to a native heart valve.

FIG. 20 illustrates a schematic cross sectional view of the prosthetic valve shown in FIG. 17 deployed to a native heart valve.

FIG. 21 illustrates a plan view of a frame of a prosthetic valve according to examples of the present disclosure.

FIG. 22 illustrates a schematic cross sectional view of the prosthetic valve utilizing the frame shown in FIG. 21 deployed to a native heart valve.

FIG. 23 illustrates a perspective view of a prosthetic valve according to examples of the present disclosure.

FIG. 24 illustrates a side view of the prosthetic valve shown in FIG. 23 being deployed.

FIG. 25 illustrates a side view of the prosthetic valve shown in FIG. 23 being deployed.

FIG. 26 illustrates a side view of the prosthetic valve shown in FIG. 23 being deployed.

FIG. 27 illustrates a plan view of a frame of a prosthetic valve according to examples of the present disclosure.

FIG. 28 illustrates a side view of a prosthetic valve utilizing the frame shown in FIG. 27 being deployed.

FIG. 29 illustrates a side view of a prosthetic valve utilizing the frame shown in FIG. 27 being deployed.

FIG. 30A illustrates a side view of a covering body covering anchoring protrusions.

FIG. 30B illustrates a side cross sectional view of the covering body shown in FIG. 30A covering anchoring protrusions.

FIG. 30C illustrates a side cross sectional view of the covering body shown in FIG. 30A being retracted from an anchoring arm.

FIG. 31A illustrates a side view of a covering body covering anchoring protrusions.

FIG. 31B illustrates a side cross sectional view of the covering body shown in FIG. 31A covering anchoring protrusions.

FIG. 31C illustrates a side cross sectional view of the covering body shown in FIG. 31A being removed from an anchoring arm.

FIG. 32A illustrates a cross sectional view of a prosthetic valve in an undeployed configuration.

FIG. 32B illustrates a cross sectional view of the prosthetic valve shown in FIG. 32A being deployed.

FIG. 32C illustrates a cross sectional view of the prosthetic valve shown in FIG. 32A deployed.

FIG. 33A illustrates a cross sectional view of a prosthetic valve.

FIG. 33B illustrates a side perspective view of a portion of the prosthetic valve shown in FIG. 33A.

FIG. 33C illustrates a side view of the prosthetic valve shown in FIG. 33A being deployed.

FIG. 33D illustrates a side view of the prosthetic valve shown in FIG. 33A being deployed.

FIG. 33E illustrates a side view of the prosthetic valve shown in FIG. 33A deployed.

FIG. 34 illustrates a schematic cross sectional view of a prosthetic valve according to examples of the present disclosure.

FIG. 35 illustrates a schematic side view of a frame that may be utilized with the prosthetic valve of FIG. 34.

FIG. 36 illustrates a schematic side view of the prosthetic valve shown in FIG. 34 within a capsule of a delivery apparatus according to examples of the present disclosure.

FIG. 37 illustrates a schematic side view of the prosthetic valve shown in FIG. 34 being deployed to a native heart valve.

FIG. 38 illustrates a schematic side view of the prosthetic valve shown in FIG. 34 being deployed to a native heart valve.

FIG. 39 illustrates a schematic side view of the prosthetic valve shown in FIG. 34 being deployed to a native heart valve.

FIG. 40 illustrates a schematic cross sectional view of a prosthetic valve according to examples of the present disclosure.

FIG. 41 illustrates a schematic side view of a frame according to examples of the present disclosure.

FIG. 42 illustrates a schematic cross sectional view of a prosthetic valve according to examples of the present disclosure.

FIG. 43 illustrates a schematic side view of the prosthetic valve shown in FIG. 42 deployed to a native heart valve.

FIG. 44 illustrates a perspective view of a frame according to examples of the present disclosure.

FIG. 45 illustrates a perspective view of a frame according to examples of the present disclosure.

FIG. 46 illustrates a perspective view of the frame shown in FIG. 44 combined with the frame shown in FIG. 45 according to examples of the present disclosure.

FIG. 47 illustrates a plan view of a frame pattern.

FIG. 48 illustrates a perspective view of the frame pattern shown in FIG. 47.

FIG. 49 illustrates a schematic cross sectional view of a frame of a prosthetic valve utilizing the frame pattern shown in FIG. 47.

FIG. 50 illustrates a schematic cross sectional view of a frame of a prosthetic valve utilizing the frame pattern shown in FIG. 47.

FIG. 51 illustrates a perspective view of a prosthetic valve according to examples of the present disclosure.

FIG. 52 illustrates a schematic cross sectional view of the prosthetic valve shown in FIG. 51.

FIG. 53 illustrates a side view of the prosthetic valve shown in FIG. 51 being deployed.

FIG. 54 illustrates a side view of the prosthetic valve shown in FIG. 51 deployed.

FIG. 55 illustrates a side view of the prosthetic valve shown in FIG. 51 being deployed.

FIG. 56 illustrates a perspective cross sectional view of a prosthetic valve according to examples of the present disclosure.

FIG. 57 illustrates a perspective view of the prosthetic valve shown in FIG. 56.

FIG. 58 illustrates a perspective cross sectional view of a prosthetic valve according to examples of the present disclosure.

FIG. 59 illustrates a perspective cross sectional view of a prosthetic valve according to examples of the present disclosure.

FIG. 60 illustrates a bottom perspective view of a frame for a prosthetic valve according to examples of the present disclosure.

FIG. 61 illustrates a side view of a frame for a prosthetic valve according to examples of the present disclosure.

FIG. 62 illustrates a perspective view of the outer frame shown in FIG. 61 interlaced with the inner frame shown in FIG. 61 according to examples of the present disclosure.

FIG. 63 illustrates a side view of a frame for a prosthetic valve according to examples of the present disclosure.

FIG. 64 illustrates a top view of the frame shown in FIG. 63.

FIG. 65 illustrates a schematic cross sectional view of the frame shown in FIG. 63 being crimped.

FIG. 66 illustrates a schematic cross sectional view of the frame shown in FIG. 63 being crimped.

FIG. 67 illustrates a schematic cross sectional view of the frame shown in FIG. 63 crimped.

FIG. 68 illustrates a schematic cross sectional view of a delivery apparatus approaching a left ventricle according to examples of the present disclosure.

FIG. 69 illustrates a schematic cross sectional view of a frame of a prosthetic valve being deployed to a mitral valve according to examples of the present disclosure.

FIG. 70 illustrates a schematic cross sectional view of a frame of a prosthetic valve being deployed to a mitral valve according to examples of the present disclosure.

FIG. 71 illustrates a schematic cross sectional view of a prosthetic valve deployed to a mitral valve according to examples of the present disclosure.

FIG. 72 illustrates a schematic cross sectional view of the prosthetic valve shown in FIG. 71 being assembled.

FIG. 73 illustrates a schematic cross sectional view of the prosthetic valve shown in FIG. 71 assembled.

FIG. 74 illustrates a schematic view of a frame positioned within an electropolishing bath.

FIG. 75 illustrates a side schematic view of an anchor positioned within an electropolishing bath.

FIG. 76 illustrates a side cross sectional view of an anchor extending around a native valve leaflet.

FIG. 77 illustrates a schematic view of an anchor positioned within an electropolishing bath.

FIG. 78 illustrates a side schematic view of an anchor.

FIG. 79 illustrates a perspective schematic view of a body within an electropolishing bath.

FIG. 80 illustrates a perspective view of a prosthetic valve.

FIG. 81 illustrates a bottom perspective view of the prosthetic valve shown in FIG. 80.

FIG. 82 illustrates a side cross sectional view of the prosthetic valve shown in FIG. 80.

FIG. 83 illustrates a side cross sectional view of the prosthetic valve shown in FIG. 80 deployed to a native heart valve.

FIG. 84 illustrates a side cross sectional view of the prosthetic valve shown in FIG. 80 deployed to a native heart valve.

FIG. 85 illustrates a side cross sectional view of the prosthetic valve shown in FIG. 80 deployed to a native heart valve.

FIG. 86 illustrates a side cross sectional view of a prosthetic valve deployed to a native heart valve.

FIG. 87 illustrates a perspective view of a prosthetic valve.

FIG. 88 illustrates a side cross sectional view of an anchor.

FIG. 89 illustrates a side cross sectional view of an anchor.

FIG. 90 illustrates a schematic side view of a delivery apparatus approaching a native heart valve.

FIG. 91 illustrates a schematic side view of a prosthetic valve being deployed to a native heart valve.

FIG. 92 illustrates a schematic side view of a prosthetic valve being deployed to a native heart valve.

FIG. 93 illustrates a schematic side view of a prosthetic valve deployed to a native heart valve.

FIG. 94 illustrates a schematic side view of a delivery apparatus approaching a native heart valve.

FIG. 95 illustrates a schematic side view of a prosthetic valve being deployed to a native heart valve.

FIG. 96 illustrates a schematic side view of a prosthetic valve being deployed to a native heart valve.

FIG. 97 illustrates a schematic side view of a prosthetic valve being deployed to a native heart valve.

FIG. 98 illustrates a schematic side view of a prosthetic valve deployed to a native heart valve.

FIG. 99 illustrates a schematic side view of a delivery apparatus approaching a native heart valve.

FIG. 100 illustrates a schematic side view of a prosthetic valve being deployed to a native heart valve.

FIG. 101 illustrates a schematic side view of a prosthetic valve being deployed to a native heart valve.

FIG. 102 illustrates a schematic side view of a prosthetic valve being deployed to a native heart valve.

### DETAILED DESCRIPTION

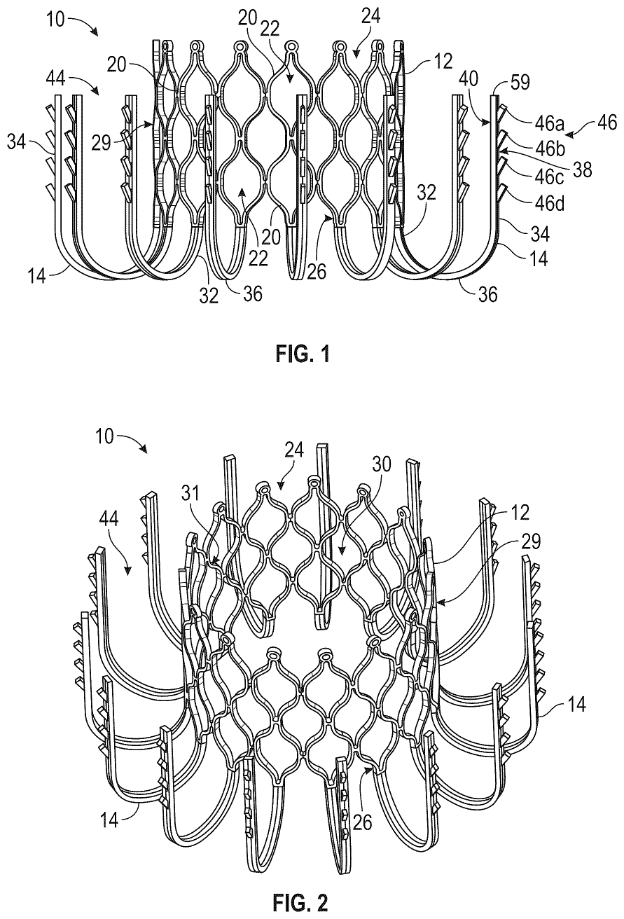

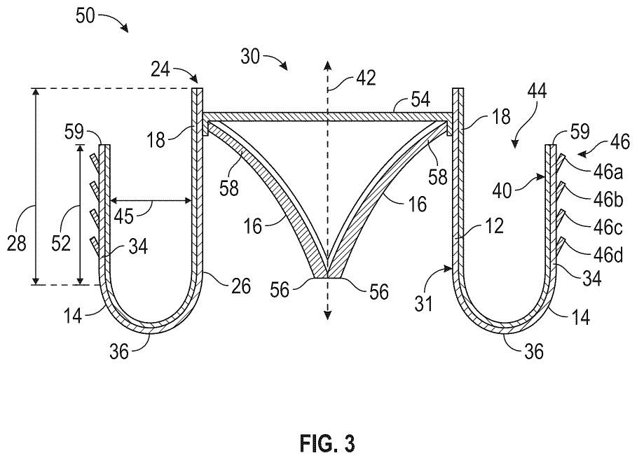

FIG. 1 illustrates a side view of a frame 10 of a prosthetic valve that may be utilized according to examples herein. The frame 10 may include a valve frame 12 and a plurality of elongate anchor arms 14. The prosthetic valve may include additional features as shown in FIG. 3, for example, including a plurality of prosthetic valve leaflets 16 and a sealing skirt 18, which are excluded from view in FIGS. 1 and 2 for clarity.

The valve frame 12 may be configured to support the plurality of prosthetic valve leaflets 16. The valve frame 12 may include a plurality of struts 20 that are connected to each other and may include openings 22 therebetween. The plurality of struts 20 may form a lattice structure with struts 20 connected at junctures between the struts 20. The valve frame 12 may comprise an expandable valve frame. The lattice structure, for example, may be configured to allow the valve frame 12 to expand from an undeployed configuration radially outward to a deployed configuration.

The valve frame 12 may include a proximal end portion 24 and a distal end portion 26 and may have a length 28 (marked in FIG. 3) between the proximal end portion 24 and the distal end portion 26. The valve frame 12 may include an outer surface 29 configured to face radially outward and may include an inner surface 31 configured to face radially inward.

The valve frame 12 may have a cylindrical shape in examples. The valve frame 12 may surround a central channel 30 for fluid flow, which may pass through the valve frame 12 between the proximal end portion 24 and the distal end portion 26. The inner surface 31 may face radially inward in examples and the outer surface 29 may face radially outward. The proximal end portion 24 in examples may comprise an inlet portion and the distal end portion 26 in examples may comprise an outlet portion based on the direction of flow allowed by the prosthetic valve leaflets 16 shown in FIG. 3. In examples, the direction of flow by the prosthetic valve leaflets 16 may allow the proximal end portion 24 to comprise an outlet portion and the distal end portion 26 to comprise an inlet portion, based on the intended use of the prosthetic valve. For example, in an example in which a mitral or tricuspid valve delivery is intended, the proximal end portion 24 may comprise an inlet portion and the distal end portion 26 may comprise an outlet portion. In an example in which an aortic or pulmonary valve delivery is intended, the distal end portion 26 may comprise an inlet portion and the proximal end portion 24 may comprise an outlet portion. Other configurations may be utilized for other delivery locations as desired.

In examples, the valve frame 12 may have another configuration as desired.

The plurality of elongate anchor arms 14 may each have a first end portion 32 coupled to the valve frame 12 and each configured to extend radially outward from the valve frame 12 to a second end portion 34 that is configured to anchor to an interior surface of a heart valve. The second end portion 34 may be positioned radially outward from the valve frame 12 in a deployed configuration. The first end portions 32 of the elongate anchor arms 14 may be coupled to the distal end portion 26 of the valve frame 12 and may extend radially outward from the distal end portion 26 of the valve frame 12. The elongate anchor arms 14, for example, may each couple to distal struts 20 of the valve frame 12, which may comprise the distalmost struts of the valve frame. As shown in FIG. 1, the first end portions 32 may couple to the apices of the valve frame 12 and extend distally from the apices. In examples, other locations of coupling may be utilized, for example, to other junctures of the valve frame 12 or other portions of struts 20. In examples, the plurality of elongate anchor arms 14 may be formed integral with the valve frame 12 and may extend distally from the distal apices of the valve frame 12. The valve frame 12 and the elongate anchor arms 14 may form an integral structure.

Bend portions 36 may be positioned between the first end portions 32 and the second end portions 34. The bend portions 36 may extend radially outward from the first end portions 32 and may be configured to direct the second end portion 34 to extend in a different direction than the first end portions 32. The first end portions 32 as shown in FIG. 1 may extend in a first direction and the bend portion 36 is configured to direct the second end portion 34 to extend in a second direction that is opposed to the first direction. The bend portion 36 may angle the second end portion 34 to extend in the proximal direction in the deployed configuration. The first direction, for example, may be a distal direction and the second direction may be a proximal direction as shown in FIG. 1. The bend portion 36 may comprise a curved portion, which may have a convex shape directed distally as shown in FIG. 1. In examples, other configurations of bend portions may be utilized, including angled configurations or other configurations as desired. The bend portions 36 may space the second end portions 34 radially from the first end portions 32. Each of the plurality of anchor arms 14 may hook due to the bend portions 36. Each elongate anchor arm 14 may have a hook shape in a deployed configuration.

The second end portions 34 may extend from the bend portions 36 and may extend proximally as shown in FIG. 1. The second end portions 34 may extend straight as shown in FIG. 1 or may have other configurations such as curvatures or angled portions as desired. The second end portions 34 may extend parallel with the outer surface 29 of the valve frame 12 in examples or may be angled with respect to the outer surface 29 in examples. The second end portions 34 may each include a radially outward facing surface 38 and a radially inward facing surface 40. The radially outward facing surface 38 may comprise a surface configured to anchor to an interior surface of a heart valve, and the radially inward facing surface 40 may comprise a surface configured to face towards the valve frame 12. In examples, second end portions 34 extending parallel with the outer surface 29 or with a central axis 42 (marked in FIG. 3) of the prosthetic valve may increase the surface area of anchoring engagement between the second end portions 34 and the interior surface of the heart valve.

The plurality of elongate anchor arms 14 may be circumferentially spaced from each other. The spacing may be an equal spacing as shown in FIG. 2 for example or varied spacing may be utilized as desired.

The second end portions 34 may be arranged to form a ring about the valve frame 12. Each of the second end portions 34 may collectively form an outward facing surface for the prosthetic valve that is radially spaced from the valve frame 12 and extends circumferentially about the valve frame 12 and anchors to the interior surface of the heart valve.

The second end portions 34 may be radially spaced from the valve frame 12 to form an annular gap 44 between the outer surface 29 of the valve frame 12 and the ring formed by the second end portions 34. The annular gap 44 may be bounded in the distal direction by the bend portions 36 and may be open at the proximal portion of the annular gap 44. The ring may have a diameter that is larger than a diameter of the valve frame 12 such that the annular gap 44 is provided between the second end portions 34 of the elongate anchor arms 14 and the valve frame 12.

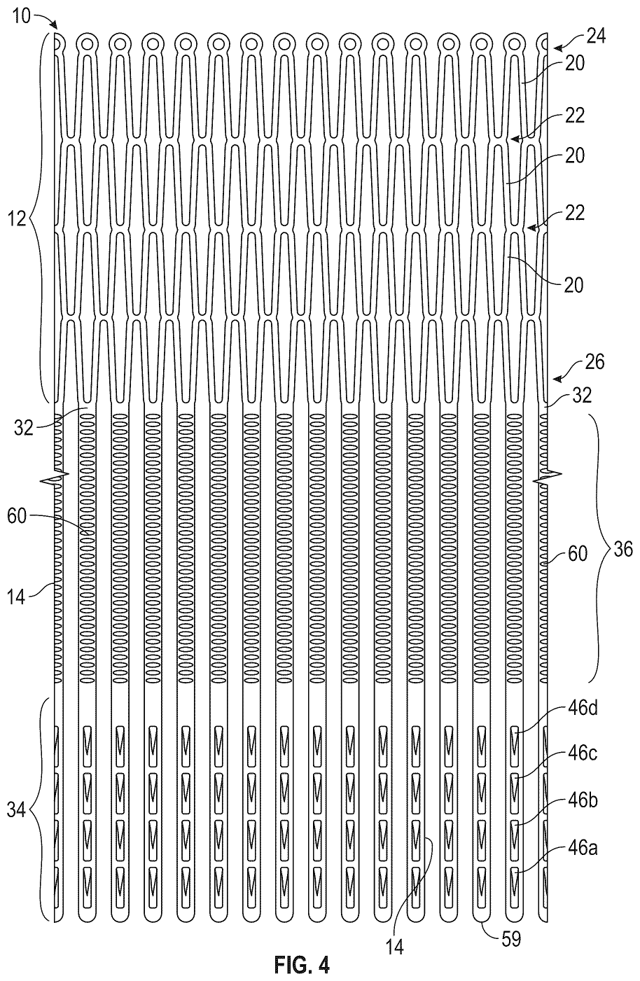

In examples, each of the plurality of elongate anchor arms 14 may be configured to deflect radially inward to accommodate a shape of an interior surface of a heart valve to which the prosthetic valve is deployed. For example, each of the plurality of elongate anchor arms 14 may be flexible to allow for a radial distance 45 (marked in FIG. 3) between a respective anchor arm 14 and the valve frame 12 to vary. In examples, the bend portions 36 may be configured to deflect to provide for such flexibility, and in examples the bend portions 36 may include one or more cuts 60 (shown in FIG. 4) to provide for such flexibility. Each of the elongate anchor arms may be independent from the other elongate anchor arms. Each of the plurality of elongate anchor arms 14 may be configured to deflect independent of each other, to allow for a variety of shapes of the resulting ring about the valve frame 12 formed by the elongate anchor arms 14. For example, with a circular heart valve, the ring may form a circular shape as shown in FIGS. 1-3. With a “D” shaped valve or ovoid shaped valve, the ring may deform to accommodate such a shape (e.g., form a respective “D” shaped valve or ovoid shaped valve). The shape of the valve frame 12 may be remain circular as shown in FIGS. 1 and 2 in examples with the configuration of the anchor arms 14 varying.

Each of the second end portions 34 may be configured to anchor to an interior surface of the heart valve. The anchoring may have a variety of forms including frictional engagement with the interior surface of the heart valve, or anchoring features that may be utilized to anchor to the interior surface of the heart valve. The anchoring features may have a variety of forms. The anchoring features may comprise apertures configured to receive sutures or the like for anchoring to the interior surface. In examples, the anchoring features may comprise one or more anchoring protrusions 46. Each of the plurality of elongate anchoring arms 14 may include one or more anchoring protrusions 46 for engaging the interior surface of the heart valve. The anchoring protrusions 46 may be provided along an outer surface of each elongate anchor arm 14. The anchoring protrusions 46 may be shaped for engaging a leaflet of the native heart valve. The anchoring protrusions 46 may be positioned on the radially outward facing surface 38 of the second end portions 34 in examples. The anchoring protrusions 46 may extend radially outward from the second end portions 34 in examples. Anchoring protrusions 46a-d are marked in FIG. 1.

The anchoring protrusions 46 may be configured to engage the interior surface of the heart valve by puncturing or otherwise gripping the interior surface of the heart valve. For example, one or more leaflets or an annulus of the heart valve may be punctured by one or more of the anchoring protrusions to anchor the prosthetic valve to the interior surface of the heart valve. The anchoring protrusions may comprise barbs or may have another configuration in examples. The positions of the anchoring protrusions 46a-d on the anchoring arms may produce circumferential anchoring for the prosthetic valve with the interior surface of the heart valve.

In examples, the anchoring protrusions 46 may be angled, and may be angled proximally as shown in FIG. 1 for example. The proximal angle, in examples, may increase the anchoring of the prosthetic valve opposite the inflow direction of the prosthetic valve (for example in a mitral or tricuspid implementation). The direction of angle accordingly may oppose a force that a ventricle may apply to the prosthetic valve. The anchoring protrusions 46 may extend in a distal direction in an undeployed configuration and may extend in a proximal direction in a deployed configuration. In examples, other angles of anchoring and configurations of anchors may be utilized as desired.

Four anchoring protrusions 46a-d may be utilized in examples as shown in FIG. 1, although in other examples other numbers (e.g., at least 1, 2, 3, 4, 5, or greater, may be utilized in examples). In examples, other forms of anchoring may be utilized, not limited to anchoring protrusions.

FIG. 2 illustrates a top perspective view of the frame 10 shown in FIG. 1. FIG. 3 illustrates a schematic cross sectional view of a prosthetic valve 50 utilizing the frame 10 shown in FIGS. 1 and 2. The elongate anchor arms 14 are shown to extend radially outward from the valve frame to form the annular gap 44 between the outer surface 29 of the valve frame 12 and the plurality of elongate anchor arms 14.

In examples, the second end portions 34 may have a length 52 that is less than the length 28 of the valve frame 12 between the proximal end portion 24 and the distal end portion 26. In examples, the length 52 may be the same or greater than the length 28 of the valve frame 12.

The valve frame 12 may support the plurality of prosthetic valve leaflets 16 within the central channel 30. The prosthetic valve leaflets 16 may extend radially inward of the inner surface 31 of the valve frame 12 and may be aligned with the central axis 42 and may surround the central axis 42 as shown in FIG. 3. A support body 54 may be coupled to the prosthetic valve leaflets 16 for example and may couple the prosthetic valve leaflets 16 to the inner surface 31 of the valve frame 12. The support body 54 may comprise an intermediate body that couples to the prosthetic valve leaflets 16 via sutures or another form of coupler, and couples to the valve frame 12 via sutures or another form of coupler as well. In examples, the prosthetic valve leaflets 16 may be coupled directly to the valve frame 12.

The prosthetic valve leaflets 16 may be positioned within the central channel 30 such that the prosthetic valve leaflets 16 are surrounded by the valve frame 12 and the second end portions 34 of the plurality of anchors 14, as shown in FIG. 3. In examples, the prosthetic valve leaflets 16 may have their length vary or may be axially displaced relative to the second end portions 34 of the plurality of anchors 14 such that another configuration results. Distal ends 56 of the prosthetic valve leaflets 16 may be positioned proximal of the bend portions 36 in examples, and proximal ends 58 of the prosthetic valve leaflets 16 may be positioned proximal of tips 59 of the elongate anchors 14 in examples, although other configurations may be utilized in examples.

A sealing skirt 18 may extend along the valve frame 12 and along the plurality of elongate anchor arms 14 in examples. The sealing skirt 18, for example, may extend from the proximal end portion 24 of the valve frame 12 distally to the distal end portion 26 of the valve frame 12. The sealing skirt 18 may extend from the distal end portion 26 of the valve frame 12 along the bend portions 36 and along the second end portions 34 of the plurality of elongate anchor arms 14. The sealing skirt 18 may extend to the tips 59 of the elongate anchor arms 14 in examples. The sealing skirt 18 may form an annular covering extending circumferentially about the valve frame 12 and the elongate anchor arms 14.

The sealing skirt 18 may extend along the outer surface 29 of the valve frame 12 in examples and along the radially inward facing surface 40 of the plurality of elongate anchor arms 14. In examples, the sealing skirt 18 may extend along the inner surface 31 of the valve frame 12 and the radially outward facing surface 38 of the elongate anchor arms 14. In such an example, the anchoring protrusions 46 may protrude through the sealing skirt 18 or the sealing skirt 18 may be excluded from extending to the anchoring protrusions 46, although other configurations may be utilized as desired. The sealing skirt 18 may be flexible in examples.

The sealing skirt 18 may extend over the plurality of elongate anchor arms 14 for providing a seal and preventing regurgitation. The sealing skirt 18 may be configured to form a seal with the interior surface of the heart valve and may reduce the possibility of fluid flowing outside of the flow path provided by the prosthetic valve leaflets 16. In examples, the configuration of the sealing skirt 18 may vary from the configuration shown in FIG. 3. For example, the scaling skirt 18 may be configured to billow between the valve frame 12 and the elongate arms 14 in examples or may have another configuration.

The prosthetic valve 50 shown in FIG. 3 and the frame 10 shown in FIGS. 1 and 2 are in a deployed configuration. The deployed configuration may be the configuration of the prosthetic valve 50 upon deployment to a desired implantation site. In examples, the prosthetic valve 50 may be configured to move from an undeployed configuration to the deployed configuration.

FIG. 4 illustrates a plan view of the frame 10 in an undeployed configuration. In the undeployed configuration, the valve frame 12 may be radially compressed, which may reduce the diameter of the valve frame 12 and increase the length of the valve frame 12. The struts 20 are drawn towards each other as shown in FIG. 4. The width of the openings 22 has reduced and the length of the openings 22 has increased.

In the undeployed configuration, the plurality of elongate anchor arms 14 may each extend longitudinally from the first end portion 32 to the second end portion 34. Each elongate anchor arm 14 may be substantially straight in the undeployed configuration. For example, as shown in FIG. 4, the bend portions 36 may be straightened to position the second end portion 34 in axial alignment with the first end portion 32 and with the respective bend portions 36. As such, each of the plurality of elongate anchor arms 14 may extend longitudinally from the distal end portion 26 of the valve frame 12 to the tips 59 of the elongate anchor arms 14. The diameter formed between the elongate anchor arms 14 in the undeployed configuration may be less than the diameter shown in FIGS. 1-3 for example in the deployed configuration, and may be the same as the diameter of the valve frame 12 in the undeployed configuration. The first end portions 32 of the plurality of elongate anchor arms 14 may be coupled to the distal end portion 26 of the valve frame 12 in the undeployed configuration.

In examples, each of the elongate anchor arms 14 may be configured to deflect from the undeployed configuration to the deployed configuration. The plurality of elongate anchor arms 14 may be configured to move from the undeployed configuration to the deployed configuration, with the plurality of elongate anchor arms extending longitudinally from the first end portion 32 to the second end portion 34 in the undeployed configuration, with the plurality of elongate anchor arms 14 deflecting radially outward from the undeployed configuration to the deployed configuration. Each of the elongate anchor arms 14 may be bent in the deployed configuration. Each of the elongate anchor arms 14 may be configured to rotate about the bend portion 36 to move from the undeployed configuration to the deployed configuration. For example, each of the plurality of elongate anchor arms 14 may extend distally in the undeployed configuration and may be configured to rotate about the bend portion 36 to extend proximally in the deployed configuration. The rotation may be in the radially outward direction. The rotation of the elongate anchor arms 14 may result in the configuration of the elongate anchor arms 14 shown in FIGS. 1-3.

In examples, each of the bend portions 36 may include a plurality of cuts 60. The cuts 60 may be positioned along a surface of the bend portions 36 and may enhance a flexibility of the respective one of the anchor arms 14. For example, in examples the cuts 60 may be positioned on a surface of the bend portions 36 that faces radially outward in the undeployed configuration as shown in FIG. 4 and that faces proximally in the deployed configuration shown in FIGS. 1-3. The cuts 60 may enhance the ability of the bend portion to deflect and direct the second end portion 34 proximally from the undeployed configuration shown in FIG. 4. The cuts 60 may further enhance the ability of each of the elongate anchor arms 14 to deflect radially inward to accommodate a shape of an interior surface of a heart valve in examples.

The longitudinal configuration of the frame 10 may allow for a relatively narrow diameter frame 10, to reduce the overall diameter of a delivery system that may be utilized to deploy the prosthetic valve. In examples, other undeployed configurations may be utilized, for example, with the anchor arms 14 extending proximally as shown in FIGS. 1-3 in an undeployed configuration. Other configurations may be utilized in examples.

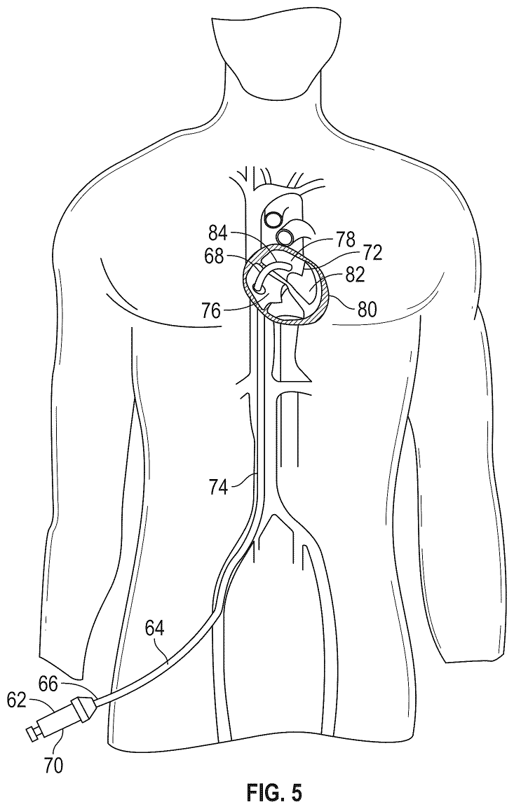

The prosthetic valve utilizing the frame 10 may be deployed for implantation utilizing a delivery apparatus. FIG. 5 for example, illustrates a configuration of a delivery apparatus 62 that may be utilized according to examples herein. The delivery apparatus 62 may include an elongate shaft 64 having a proximal end portion 66 and a distal end portion 68. The proximal end portion 66 may couple to a housing 70 such as a handle. The housing 70 may be configured to be gripped to be manipulated by a user during an implantation procedure or may have another form in examples. The elongate shaft 64 may be configured to be passed through a patient's vasculature to approach a desired implantation site. For example, as shown in FIG. 5, the elongate shaft 64 may be passed through vasculature such as a femoral vein to reach a desired implantation site. The elongate shaft 64 may pass percutaneously to the desired implantation site. Other entry methods may be utilized in examples.

The elongate shaft 64 may approach a heart valve for an implantation procedure, including a mitral valve 72 as shown in FIG. 5, or other valves as desired such as a tricuspid valve, an aortic valve, or a pulmonary valve, among other implantation sites. In examples, to approach the mitral valve 72, the elongate shaft 64 may pass through the patient's vasculature 74, through the inferior vena cava, and into the right atrium 76. In examples, the elongate shaft 64 may pass transseptal from the right atrium 76 to the left atrium 78 of the patient's heart 80 and may be directed from the left atrium 78 towards the left ventricle 82. In examples, other approaches may be utilized. For example, transapical approaches may be utilized along with other approaches to the mitral valve.

In examples, an approach to the tricuspid valve may include deflecting the elongate shaft 64 within the inferior vena cava or the right atrium 76 towards the tricuspid valve. Aortic or pulmonary implantation may include other approaches.

The distal end portion 68 of the elongate shaft 64 in examples may be configured to be deflectable to allow the distal end portion 68 to be oriented in a desired position within the patient's body. In examples, the distal end portion 68 may include an implant retention area that may retain an implant such as a prosthetic valve prior to delivery. The implant retention area, for example, may include a capsule 84 that may extend over the implant when the implant is in an undeployed configuration. The capsule 84 may retract or otherwise move relative to the implant to release the implant.

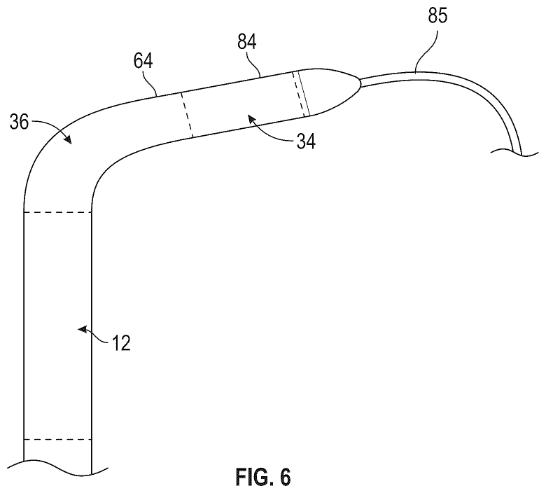

In examples, the prosthetic valve 50 may be flexible and configured to bend with the deflection of the elongate shaft 64. FIG. 6, for example, illustrates the distal end of the elongate shaft 64 deflecting along a guide wire 85. The prosthetic valve 50 (represented in dashed lines) may bend along with the elongate shaft. The bend portions 36 of the anchors arms (indicated between dashed lines) between the second end portions 34 of the anchor arms (indicated between dashed lines) and the valve frame 12 (indicated between dashed lines), for example, may comprise a more flexible portion of the prosthetic valve 50 that may be configured to bend with the elongate shaft 64. The prosthetic valve 50 may bend to reach a deployment site.

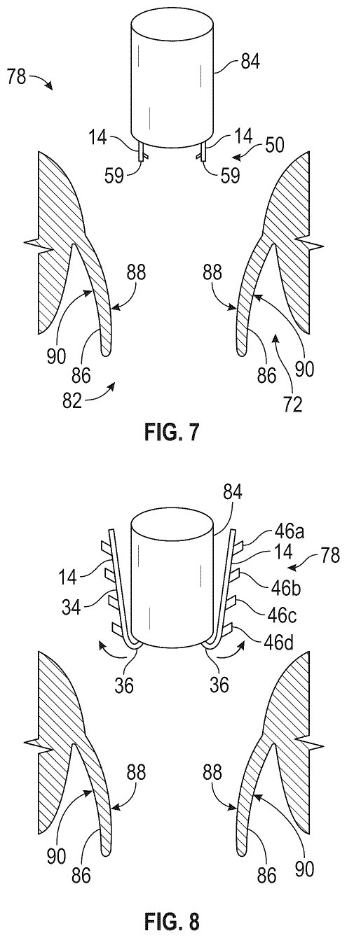

FIG. 7, for example, illustrates a deployment of the prosthetic valve 50, with the prosthetic valve 50 positioned within the capsule 84. The prosthetic valve may be in the undeployed configuration and the frame 10 may have the elongate arms 14 extended longitudinally as shown in FIG. 4 for example. The capsule 84 may approach a heart valve such as the mitral valve 72 from the left atrium 78 in a direction towards the left ventricle 82.

As the capsule 84 extends towards the mitral valve 72, the elongate arms 14 may advance distally relative to the capsule 84. Tips 59 of the elongate arms 14 may extend distally as shown in FIG. 7 and may be directed distally.

The elongate arms 14 may continue to extend distally relative to the capsule 84 until the entirety of the second end portions 34 are uncovered by the capsule 84 and the bend portions 36 may allow the elongate arms 14 to deflect proximally. FIG. 8, for example, illustrates such a configuration. The elongate arms 14 have rotated about the bend portions 36 and extend proximally. Such rotation may occur within the atrium 78 in examples or may occur within the heart valve annulus or may occur within a ventricle in examples. Various other locations may be utilized for deflection of the elongate arms 14.

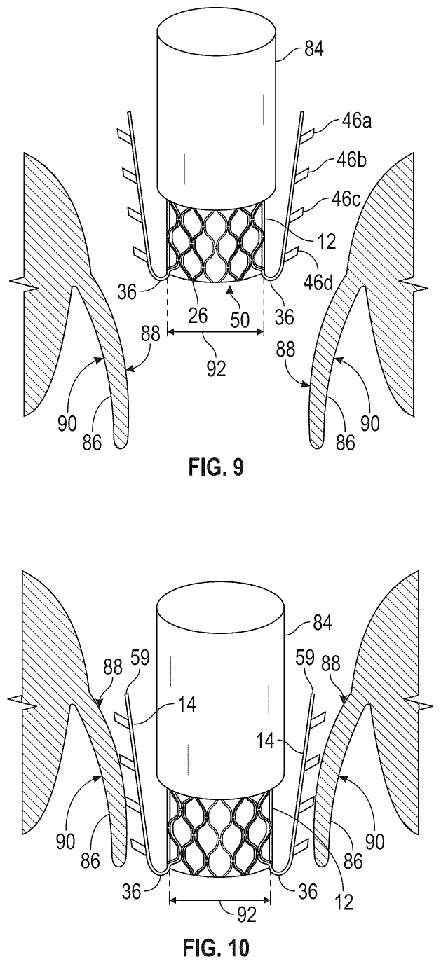

In examples, the prosthetic valve 50 may be further deployed from the capsule 84 with a portion of the valve frame 12 remaining positioned within the capsule 84. For example, the proximal end portion 24 of the prosthetic valve 50 may remain within the capsule 84 and the distal end portion 26 of the valve frame 12 may be extended distally from the capsule 84. FIG. 9, for example, illustrates the distal end portion 26 of the valve frame 12 having been advanced distally relative to the capsule 84. In such a configuration, the distal end portion 26 may be configured to expand radially outward, yet the proximal end portion 24 retained within the capsule 84 may prevent the distal end portion 26 and the valve frame 12 from fully expanding radially outward. As such, the distal end portion 26 may have a diameter 92 that is less than a fully expanded diameter of the distal end portion 26, and may result in a diameter of the elongate anchor arms 14 that is less than a fully expanded diameter of the elongate anchor arms 14 as shown in FIG. 11.

In examples, with the prosthetic valve 50 partially extended from the capsule 84, both the capsule 84 and the prosthetic valve 50 may be advanced distally as shown in FIG. 10. The prosthetic valve 50 may be positioned within the valve annulus in examples, and between the leaflets 86 of the valve 72 in examples. The prosthetic valve 50 may be positioned between the interior surfaces 88 of the valve 72 which may comprise the interior surface 88 of the leaflets 86 in examples. The interior surface 88 may face opposite an exterior surface 90 in examples.

The elongate anchor arms 14 may be positioned between the interior surface 88 of the valve 72, yet in examples may not be fully anchored to the interior surface 88. The proximal end portion 24 of the prosthetic valve 50 may remain restrained by the capsule 84 from fully expanding radially outward, thus retaining the distal end portion 26 at the diameter 92 and reducing the expansion of the elongate anchor arms 14 from fully anchoring to the leaflets 86 as shown in FIG. 10.

In a configuration as shown in FIG. 10, the prosthetic valve 50 and the desired implantation site may be imaged to determine a desired orientation of the prosthetic valve 50 relative to the implantation site. For example, the position of the prosthetic valve 50 relative to features of the valve 72 such as the valve annulus and the leaflets 86 may be determined. Imaging may include various forms of imaging, such as ultrasound (e.g., echocardiography) and/or fluoroscopy, among others. The imaging may visualize one or more features of the prosthetic valve 50, including the position of the bend portions 36 and/or the tips 59 of the anchors 14 relative to the valve 72. The axial position of the prosthetic valve 50 may be determined relative to the valve 72, and the capsule 84 and prosthetic valve 50 may be advanced distally or retracted proximally to place the prosthetic valve 50 in the desired position relative to the valve 72 prior to a full release of the prosthetic valve 50.

With the prosthetic valve 50 in the desired position, the capsule 84 may be retracted fully relative to the proximal end portion 24 of the valve frame 12 to release the proximal end portion 24. FIG. 11, for example, illustrates such a configuration with the capsule fully retracted and released from the valve frame 12. The capsule 84 may retract to allow the proximal end portion 24 to expand radially outward and allow the valve frame 12 to expand radially outward to a diameter 94 that is greater than the diameter 92 shown in FIG. 10. The expansion of the valve frame 12 may allow the elongate anchor arms 14 to expand radially outward and anchor to the interior surface 88 of the valve 72 as shown in FIG. 11. As shown in FIG. 11, the anchoring protrusions 46 may anchor to the interior surface 88 of the valve 72 by penetrating the leaflets 86, although other forms of anchoring may be utilized in examples.

The configuration of the prosthetic valve 50 may allow for axial alignment of the prosthetic valve 50 relative to the valve 72, with the prosthetic valve 50 in a reduced diameter state, and then radial expansion of the prosthetic valve 50 to a greater diameter state when the prosthetic valve 50 is in the desired orientation relative to the valve 72. The axial position of the prosthetic valve 50 may be imaged to determine the desired axial position prior to the full radial expansion of the prosthetic valve 50.

The prosthetic valve 50 may include anchors 14 configured to anchor to the interior surface 88 of the valve 72, which may allow for improved case of deployment relative to an example in which anchors may extend over the valve leaflets 86 to be positioned on the exterior surface 90 of the leaflets 86. In such configurations, imaging must often be utilized to determine if the leaflets 86 are properly captured by the anchors of such a prosthetic valve, and whether the anchors are positioned exterior of the leaflets. With a prosthetic valve as shown in FIGS. 1-11, imaging of leaflet capture may be excluded because the anchors 14 anchor to the interior surface 88 of the valve 72. A simplified deployment sequence may result.

In examples, the deployment sequence represented in FIGS. 5-11 may be varied as desired. For example, steps may be modified, excluded, substituted across examples, or otherwise varied as desired. In examples, a deployment to a tricuspid valve may include similar steps as shown in FIGS. 6-11, with the prosthetic valve 50 deployed to the tricuspid rather than mitral valve. In examples, other implantation locations may be utilized.

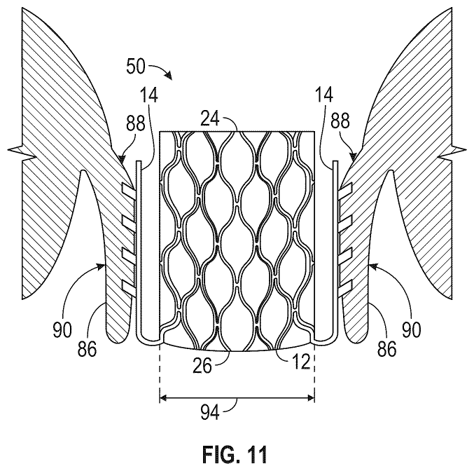

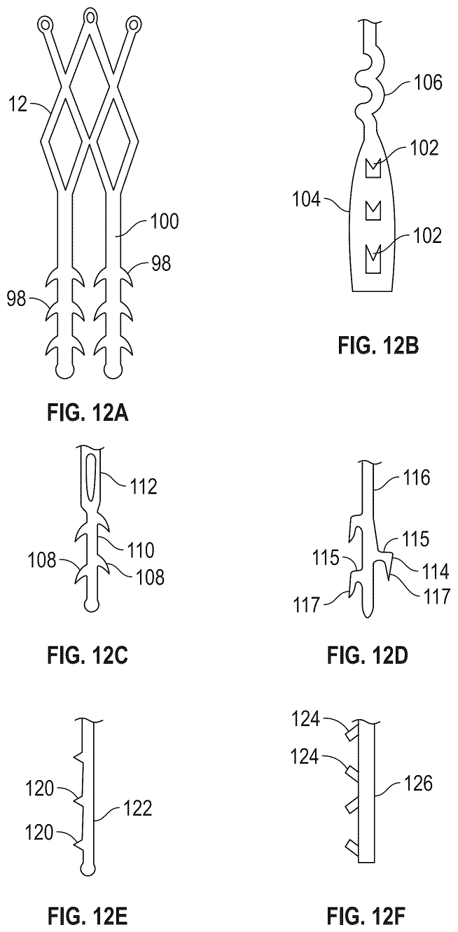

Features of the prosthetic valve 50 may be varied as desired. FIG. 12A, for example, illustrates an example in which anchoring protrusions 98 may be positioned upon elongate anchor arms 100 and may be curved. The anchoring protrusions 98 may be curved to extend proximally when the prosthetic valve is in the deployed configuration. The anchoring protrusions 98 may be angled to extend proximally. Pairs of the anchoring protrusions 98 may be horizontally aligned with each other and sets of the pairs may be spaced from each other axially.

FIG. 12B illustrates an example in which the anchoring protrusions 102 comprise cut-outs in the elongate anchor arms 104 that are configured to be bent and protrude outward from the anchor arms 104. The anchoring protrusions 102 may extend proximally when the prosthetic valve is in the deployed configuration. The anchoring protrusions 102 may be angled to extend proximally. FIG. 12B further illustrates the bend portions 106 of the elongate anchor arms 104 may include an undulating shape to allow for improved flexibility of the bend portion 106. Any example of bend portion disclosed herein may include such a shape or other configuration to provide for flexibility of the bend portion as desired.

FIG. 12C illustrates an example in which anchoring protrusions 108 may be positioned upon elongate anchor arms 110 and may be curved. The anchoring protrusions 108 may be curved to extend proximally when the prosthetic valve is in the deployed configuration. The anchoring protrusions 108 may be angled to extend proximally. Pairs of the anchoring protrusions 108 may be axially offset with each other and sets of the pairs may be spaced from each other axially. FIG. 12C further illustrates the bend portions 112 of the elongate anchor arms 110 may include an opening to allow for improved flexibility of the bend portion 112. Any example of bend portion disclosed herein may include such a shape or other configuration to provide for flexibility of the bend portion as desired.

FIG. 12D illustrates an example in which anchoring protrusions 114 may include branches 115 extending horizontally with respect to the elongate anchor arms 116 and then angled to include tips 117 extending proximally when the prosthetic valve is in the deployed configuration. The anchoring protrusions 114 may be angled to extend proximally. The anchoring protrusions 114 may be positioned axially offset with each other.

FIG. 12E illustrates an example in which anchoring protrusions 120 may be positioned upon elongate anchor arms 122 and may extend horizontally with respect to the elongate anchor arm 122. The anchoring protrusions 120 may be spaced from each other axially.

FIG. 12F illustrates an example in which anchoring protrusions 124 may be positioned upon elongate anchor arms 126 and certain of the anchoring protrusions 124 may be angled proximally and certain of the anchoring protrusions 124 may be angled distally when the prosthetic valve is in the deployed configuration. In examples, at least one of the one or more anchoring protrusions 124 may be angled proximally. At least one of the one or more anchoring protrusions 124 may be angled distally. A distally angled protrusion may resist distal movement of the prosthetic valve. Any example of anchoring protrusion disclosed herein may be angled in a distal direction, or a proximal direction, or a combination of distal and proximal directions in examples.

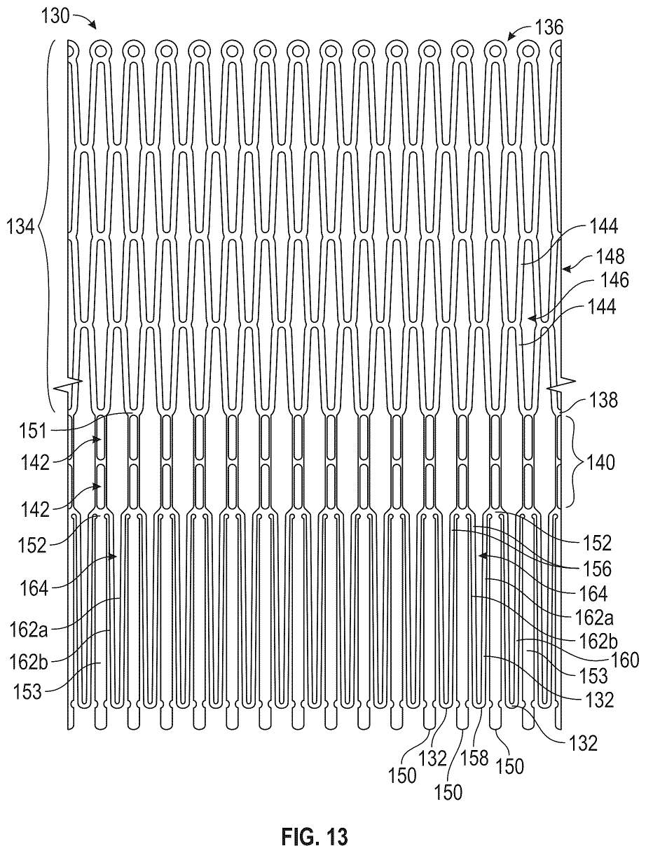

FIG. 13 illustrates an example of a frame 130 for a prosthetic valve configured similarly as the frame 10 shown in FIGS. 1-4 yet including a plurality of sealing bodies 132 configured to extend radially outward from the valve frame 134, each positioned between two anchors and configured to be positioned radially inward of a heart valve to seal fluid flow. The frame 130 may include a valve frame 134 with a proximal end portion 136 having a different diameter than the distal end portion 138 in the deployed configuration, and may include bend portions 140 having openings 142 to allow for improved flexibility of the respective bend portion 140.

The valve frame 134 may be configured similarly as the valve frame 12 shown in FIGS. 1-4 and may include a plurality of struts 144 separated by openings 146. The proximal end portion 136 of the valve frame 134, however, may have a larger diameter than the distal end portion 138 as shown in FIG. 14. A central portion 148 of the valve frame 134 for example, may have a curvature that curves radially outward in the direction from the distal end portion 138 towards the proximal end portion 136 to thus form the larger diameter at the proximal end portion 136. The valve frame 134 accordingly may have a bulb shape with the proximal end portion 136 being larger than the distal end portion 138. The valve frame 134 may have an hourglass shape in examples. Any example disclosed herein may have an hourglass shape as desired.

Referring to FIG. 13, the bend portions 140 of the anchors may include elongate openings 142 that may reduce the overall material of the bend portions 140. As such, increased flexibility of the bend portions 140 may result.

The anchors may be in the form of elongate anchor arms 150 that may be configured similarly as the elongate anchor arms 14 shown in FIGS. 1-4. The elongate anchor arms 150, for example, may be configured to anchor to an interior surface of a heart valve. Each elongate anchor arm 150 may extend from a first end portion 151 to a second end portion 153. The elongate anchor arms 150 may include a coupling portion 152 that may couple to the sealing bodies 132. The coupling portion 152, in examples, may comprise a mid-portion of each of the elongate anchor arms 150 that may be positioned between the bend portions 140 and the second end portions 153. The coupling portion 152 may have alternative locations in examples.

Each sealing body 132 may include a first end portion 156 and may extend to a second end portion 158. The first end portion 156 may be configured to couple to the coupling portion 152. The second end portion 158 may be positioned between the second end portions 153 of adjacent anchor arms 150 as shown in FIG. 13.

Each sealing body 132 may comprise a sealing strut 160 that extends from the first end portion 156 to the second end portion 158. The sealing strut 160 may have a first length 162a that extends from the first end portion 156 to the second end portion 158 and then bends at the second end portion 158 to form a second length 162b that extends from the second end portion 158 to the first end portion 156. An opening 164 may be positioned between the lengths 162a, b of the scaling strut 160.

FIG. 13 illustrates the frame 130 in an undeployed configuration in which the sealing bodies 132 and the elongate anchor arms 150 each extend longitudinally. The elongate anchor arms 150 are shown to extend distally to a greater length than the sealing bodies 132. The elongate anchor arms 150 alternate in position with the plurality of sealing bodies 132.

FIG. 14 illustrates the frame 130 in a deployed configuration with the elongate anchor arms 150 extending proximally and positioned to anchor to the interior surface of a heart valve as disclosed with respect to FIGS. 1-11. The sealing bodies 132 may deflect with the second end portions 158 of the sealing bodies 132 extending proximally. The sealing bodies 132 and elongate anchor arms 150 accordingly may form a ring extending circumferentially about the valve frame 134.

The elongate anchor arms 150 may alternate with the sealing bodies 132 circumferentially about the valve frame 134. The sealing bodies 132 may be positioned in the space between adjacent elongate anchor arms 150. The sealing bodies 132 may comprise protrusions extending radially outward from the valve frame 134. A width of the opening 164 between the lengths 162a, b may increase upon the sealing bodies 132 moving to the deployed configuration, to span the distance between the adjacent elongate anchor arms 150 and fill the space between the adjacent elongate anchor arms 150.

The sealing bodies 132 may be utilized to enhance a fluid seal with the heart valve between the positions of the adjacent elongate anchor arms 150. The sealing bodies 132, for example, may press against the interior surface of the heart valve to provide for additional sealing in the spaces between the adjacent elongate anchor arms 150.

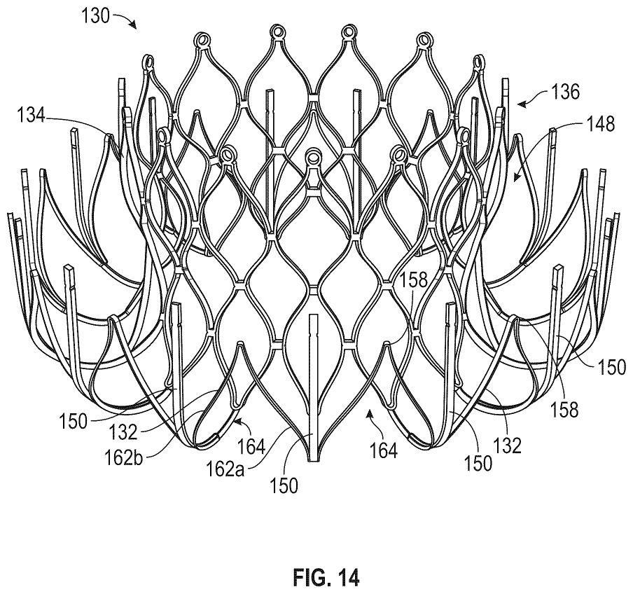

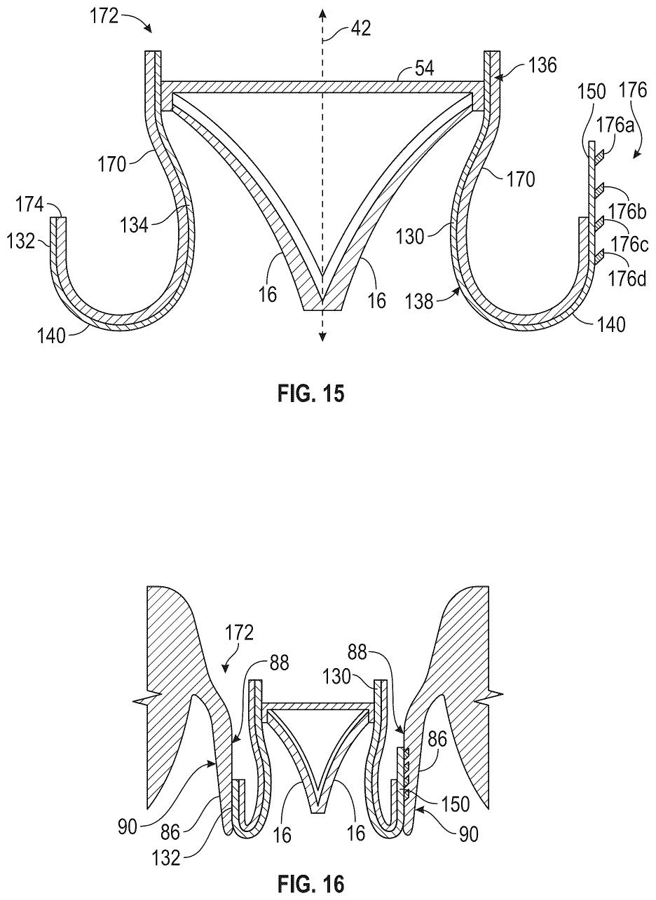

In examples, a sealing skirt 170 may be provided that may extend along the plurality of sealing bodies 132. FIG. 15 illustrates a schematic cross sectional view of a prosthetic valve 172 utilizing the frame 130 shown in FIGS. 13 and 14. The prosthetic valve 172 and the frame 130 are shown in the deployed configuration in FIG. 15. The sealing skirt 170 may extend from the proximal end portion 136 of the valve frame 134 distally to the distal end portion 138 of the valve frame 134. The sealing skirt 170 may extend from the distal end portion 138 of the valve frame 134 along the bend portions 140 and to the tips 174 of the sealing bodies 132. In examples, the sealing skirt 170 may extend to a mid-portion of the elongate anchor arms 150 as shown in FIG. 15 or may extend to a tip of the respective anchor arm 150 in a similar manner as shown in FIG. 3 for example. In examples, the sealing skirt 170 may be positioned on an interior facing surface of the valve frame 130 and an outward facing surface of the elongate anchor arms 150 and/or the sealing bodies 132. Other configurations may be utilized as desired.

The sealing skirt 170 may extend radially outward from the valve frame 134 and may form a disk that extends radially outward from the valve frame 134 and along the sealing bodies 132 and the anchor arms 150.

The anchor arms 150 may be configured similarly as the anchor arm 14 shown in FIGS. 1-4, for example, and may include a plurality of anchoring protrusions 176 (marked as anchoring protrusions 176a-d) that may be utilized to anchor to the interior surface of the heart valve.

The prosthetic valve 172 may be deployed in a similar manner as shown in FIGS. 5-11 for example. The prosthetic valve 172 may be deployed with the elongate anchor arms 150 anchoring to the interior surface 88 of the heart valve in a similar manner as shown in FIGS. 5-11. The sealing bodies 132 may contact the interior surface 88 of the heart valve to seal fluid flow between the elongate anchor arms 150. FIG. 16, for example, illustrates an exemplary deployment of the prosthetic valve 172. Improved sealing with the heart valve may result.

The features of the sealing bodies 132 of FIGS. 13-16 may be utilized with an example of FIGS. 1-12F or any other example disclosed herein.

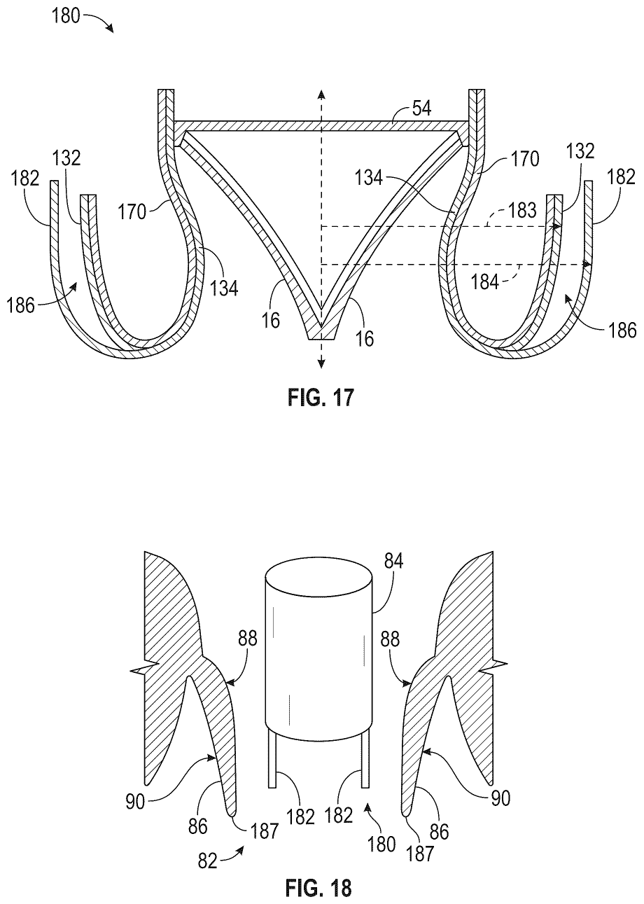

FIG. 17 illustrates an example of a prosthetic valve 180 in which anchors 182 are configured to be positioned radially outward from the valve frame 134 further than the sealing bodies 132. The prosthetic valve 180 may be otherwise configured similarly as the prosthetic valve 172 shown in FIG. 15.

The anchors 182 may be configured to extend radially outward from the valve frame 134 and be positioned radially outward of a heart valve to anchor to the heart valve. The anchors 182, for example, may comprise elongate anchor arms that are shaped to extend radially outward further than the anchor arms 150 shown in FIG. 14 and configured to hook around distal tips of valve leaflets 86 to be positioned exterior of the exterior surface 90 of the valve leaflets 86.

In examples, the anchors 182 may alternate in position with the sealing bodies 132 as shown in FIG. 14. The anchors 182, however, may extend radially outward from the position shown in FIG. 14 to a greater radius 184 than the radius 183 of the sealing bodies 132 as marked in FIG. 17. The sealing bodies 132 may each be positioned between two of the plurality of anchors 182 and may be configured to be positioned radially inward of the heart valve to seal fluid flow, which may be in a similar manner as disclosed with respect to the sealing bodies 132 of FIGS. 13-16. An area 186 that may be formed between the anchors 182 and the sealing bodies 132 may be ring shaped and may extend around the prosthetic valve 180 between the anchors 182 and the sealing bodies 132. The scaling bodies 132 and anchors 182 may be configured to position a portion of a heart valve within the area 186 upon deployment of the prosthetic valve 180. The portion of the heart valve may be valve leaflets in examples.

In examples, the sealing bodies 132 and anchors 182 may clamp the portion of the heart valve between the anchors 182 and the sealing bodies 132. The anchors 182 may be positioned radially outward of the heart valve and the sealing bodies 132 may be positioned radially inward of the heart valve, with the portion of the heart valve clamped between the anchors 182 and the sealing bodies 132. The anchors 182 may anchor the prosthetic valve 180 to the heart valve by extending over distal tips 187 of valve leaflets 86 and being positioned exterior of the exterior surface 90 of the valve leaflets 86, with the sealing bodies 132 providing a seal against the interior surface 88 of the valve leaflets 86.

FIG. 18 illustrates a deployment of the prosthetic valve 180, in which a capsule 84 advances distally towards the ventricle 82. The prosthetic valve 180 advances relative to the capsule 84, with the anchors 182 protruding from the capsule 84. The anchors 182 may be deployed distally from the capsule 84 to hook around the distal tips 187 of the valve leaflets 86 and be positioned exterior of the exterior surface 90 of the leaflets 86. The sealing bodies 132 may be deployed to be positioned interior of the interior surface 88 of the leaflets 86.

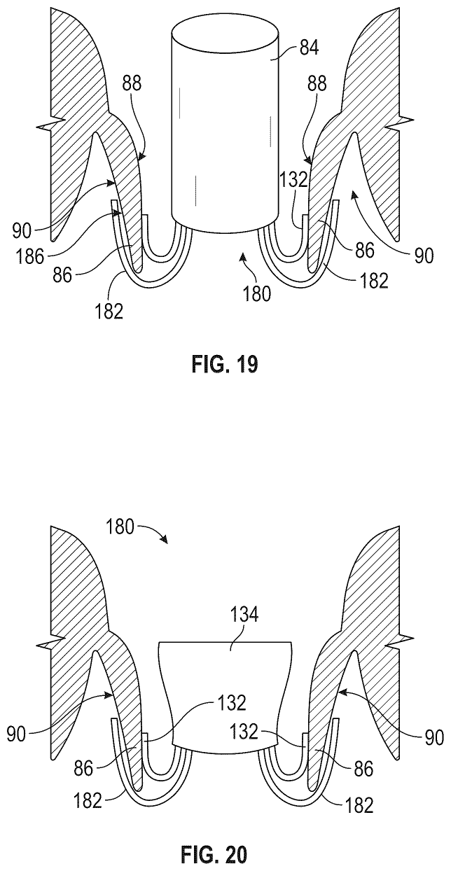

FIG. 19 illustrates the prosthetic valve 180 being deployed from the capsule 84 with the anchors 182 positioned exterior of the exterior surface 90 of the leaflets 86 and the sealing bodies 132 positioned interior of the interior surface 88 of the leaflets 86. The leaflets 86 may be clamped in the area 186 between the sealing bodies 132 and the anchors 182. With the sealing bodies 132 and anchors 182 in the desired position, the capsule 84 may be fully retracted relative to the prosthetic valve 180.

FIG. 20 illustrates the prosthetic valve 180 deployed to the implantation site.

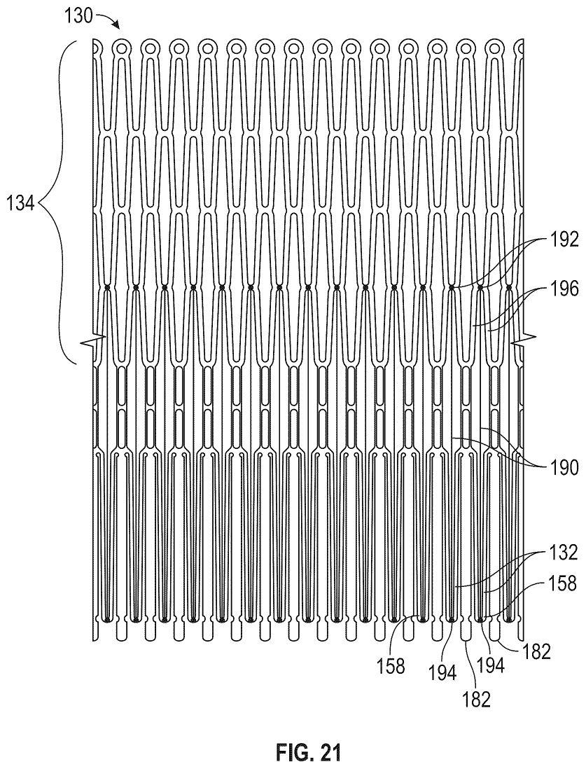

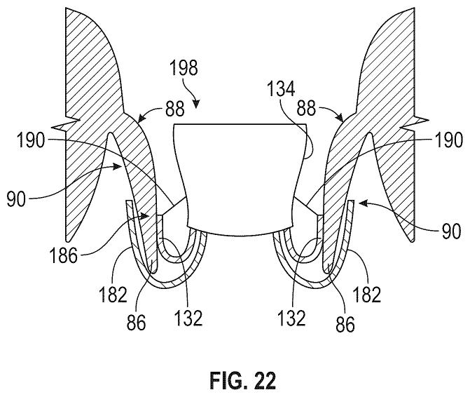

FIGS. 21-22 illustrate a modification of the prosthetic valve 180 including one or more tethers 190 configured to couple the sealing bodies 132 to the valve frame 134 and configured to vary a height of the sealing bodies 132. The tethers 190, for example, may have a first end portion 192 coupled to the valve frame 134 and a second end portion 194 coupled to the sealing bodies 132.

The first end portion 192 of the tethers 190 may couple to the struts 196 of the valve frame 134 and may couple to a junction of the struts 196 of the valve frame 134. The junction may be a distal junction of the struts 196 as shown in FIG. 21 or other junctions or portions of the struts 196 may comprise coupling points in examples. The first end portion 192 of the tethers 190 may couple to a portion of the struts 196 that are configured to move as the valve frame 134 expands. The coupling point to the struts 196 may move longitudinally as the valve frame 134 is expanded and the height of the valve frame 134 decreases. As such, the coupling point to the struts 196 may move longitudinally distal to allow a height of the sealing bodies 132 to reduce. The one or more tethers 190 accordingly may be configured to reduce a height of the plurality of scaling bodies 132 upon the height of the valve frame 134 decreasing.

The second end portion 194 of the tethers 190 may be coupled to a portion of the sealing bodies 132 such as the tip of the sealing body 132 shown in FIG. 21 or another location as desired. The one or more tethers 190 may allow the sealing bodies 132 to delay contact with the interior surface 88 of the valve leaflets 86. Contact may be delayed until full deployment is achieved. Such a feature may allow the valve leaflets 86 to better fit within the area 186 between the sealing bodies 132 and the anchors 182 shown in FIG. 17 because the size of the area 186 may be increased prior to contact between the sealing bodies 132 and the interior surface 88. The one or more tethers 190, for example, may pull the second end portions 158 radially inward until a desired time to allow the tethers 190 to extend radially outward further and reduce the height of the sealing bodies 132. The reduced height of the sealing bodies 132 may cause the sealing bodies 132 to contact the interior surface 88 of the valve leaflets 86.

The height of the sealing bodies 132 may be controlled via the radial expansion of the valve frame 134. For example, as the diameter of the valve frame 134 increases, the height of the valve frame 134 may decrease. The reduced height of the valve frame 134 may lower the tethers 190 and cause the sealing bodies 132 to reduce in height and contact the interior surface 88 of the valve leaflets 86 at a later time than they may without the use of the tethers 190.

FIG. 22 illustrates a prosthetic valve 198 utilizing the frame 130 with the tethers 190 shown in FIG. 21. The tethers 190 have lowered the height of the sealing bodies 132 to contact the interior surface 88 of the heart valve. The heart valve may be clamped between the anchors 182 and the sealing bodies 132.

The features of the examples of FIGS. 17-22 may be utilized with any other example disclosed herein.

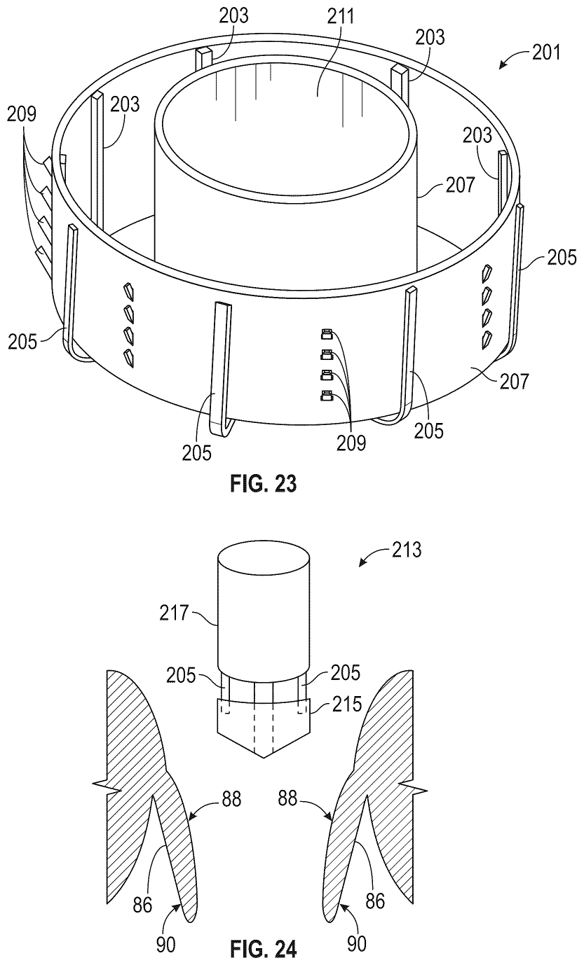

FIGS. 23-26 illustrate an example of a prosthetic valve 201 including a first plurality of elongate anchor arms 203 each configured to anchor to an interior surface of a heart valve. The prosthetic valve 201 may include a second plurality of elongate anchor arms 205 that are each configured to hook over a distal tip of a leaflet of the heart valve.

The first plurality of elongate anchor arms 203 may be configured similarly as the elongate anchor arms 14 discussed with respect to FIGS. 1-4, for example. A sealing skirt 207 is shown in the perspective view of FIG. 23 and may be positioned on the outer surfaces of the first plurality of elongate anchor arms 203, with the anchoring protrusions 209 extending through the sealing skirt 207. The sealing skirt 207 may extend to and cover the valve frame 211. In examples, the sealing skirt 207 may be positioned on an interior facing surface of the first plurality of elongate anchor arms 203.

The second plurality of elongate anchor arms 205 may have a similar shape as the first plurality of elongate anchor arms 203 yet may be configured to be positioned radially outward of the first plurality of elongate anchor arms 203. The second plurality of elongate anchor arms 205 may be configured to move independent of the first plurality of elongate anchor arms 203. For example, the second plurality of elongate anchor arms 205 may be configured to rotate and bend from an undeployed configuration to a deployed configuration independent of the first plurality of elongate anchor arms 203.

At least one of the second plurality of elongate anchor arms 205 may be positioned between two of the first plurality of elongate anchor arms 203. The position of the second plurality of elongate anchor arms 205 may alternate with the first plurality of elongate anchor arms 203 as shown in FIG. 23, or other configurations may be utilized in examples as desired.

In deployment, the second plurality of elongate anchor arms 205 may be configured to be deployed to a position radially outward of a heart valve leaflet, and the first plurality of elongate anchor arms 203 may be configured to deployed to an interior surface of the heart valve leaflet. The second plurality of elongate anchor arms 205 accordingly may provide additional anchoring against a force in a proximal or atrial direction.

FIGS. 24-26 illustrate an exemplary deployment sequence for the prosthetic valve 201. Referring to FIG. 24, a delivery apparatus 213 may approach an implantation site, such as a mitral heart valve, although other implantation sites may be utilized as desired. The delivery apparatus 213 may be configured to retain the second plurality of elongate anchor arms 205 independent from the first plurality of elongate anchor arms 203. The delivery apparatus 213, for example, may be configured to deploy the second plurality of elongate anchor arms 205 independent from the deployment of the first plurality of elongate anchor arms 203.

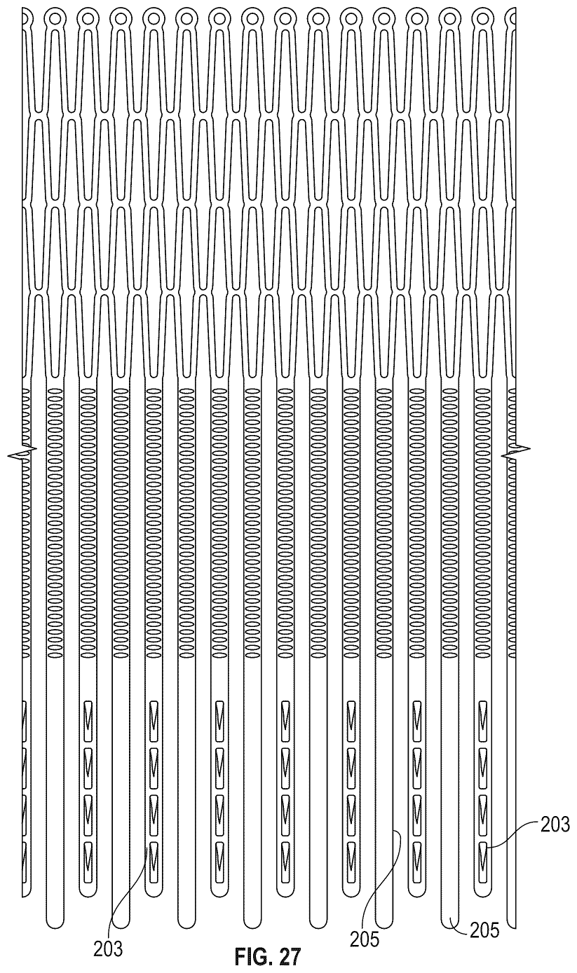

The delivery apparatus 213, for example, may include a retaining body 215 that retains the second plurality of elongate anchor arms 205 independent from a retaining body such as a capsule 217 that may retain the first plurality of elongate anchor arms 203. In the example shown in FIGS. 24-26, the first plurality of elongate anchor arms 203 may be retained extending proximally in an undeployed configuration, although other forms of retention may be utilized as desired to retain the first plurality of elongate anchor arms 203 in an undeployed configuration extending distally. FIGS. 27-29, for example, illustrate an example in which a first plurality of elongate anchor arms are retained in an undeployed configuration extending distally.

FIG. 24 illustrates the retaining body 215 as a cap of the delivery apparatus 213 that may be movable independent of the capsule 217. Other forms of retaining bodies such as sutures, or movable arms may be utilized as desired. The retaining body 215 may retain the second plurality of elongate anchor arms 205.

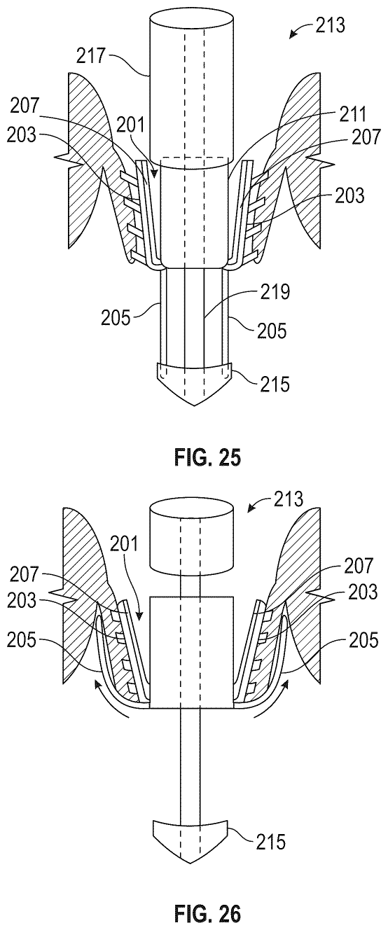

FIG. 25 illustrates the delivery apparatus 213 having deployed the first plurality of elongate anchor arms 203 such that the first plurality of elongate anchor arms 203 are positioned radially inward of the native valve leaflets. The capsule 217, for example, has retracted to deploy the first plurality of elongate anchor arms 203. The elongate anchor arms 203 may fully or partially anchor to the interior surfaces of the native heart valve leaflets or may not yet be anchored to the interior surfaces.

The retaining body 215 may retain the second plurality of elongate anchor arms 205 in an undeployed configuration. For example, FIG. 25 illustrates the second plurality of elongate anchor arms 205 straightened and positioned distal of the distal tips of the native heart valve leaflets. The retaining body 215, for example, may be coupled to an inner shaft 219 that may extend distally from the distal end of the capsule 217 and may be slidable relative to the capsule 217 and the prosthetic valve 201. At a desired time, the retaining body 215 may be advanced distally relative to the second plurality of elongate anchor arms 205 to release the arms 205 and allow the arms 205 to rotate to hook around the distal tips of the leaflets.

FIG. 26, for example, illustrates the retaining body 215 having been advanced distally relative to the second plurality of elongate anchor arms 205 to allow the arms 205 to rotate over the leaflets. The arms 205 may be positioned radially outward of the leaflets. The first plurality of elongate anchor arms 203 may fully anchor to the interior surfaces of the leaflets. The delivery apparatus 213 may be retracted with the prosthetic valve 201 remaining in position.

The features of the prosthetic valve and delivery system shown in FIGS. 23-26 may be utilized solely or in combination with any other example disclosed herein.

In examples, the lengths of the first plurality of elongate anchor arms 203 may be varied from the second plurality of elongate anchor arms 205. FIG. 27, for example, illustrates such a flat pattern of such a configuration, with the second plurality of elongate anchor arms 205 being longer than the first plurality of elongate anchor arms 203 and the tips of the first plurality of anchor arms 203 extending further than the second plurality of elongate anchor arms 205.

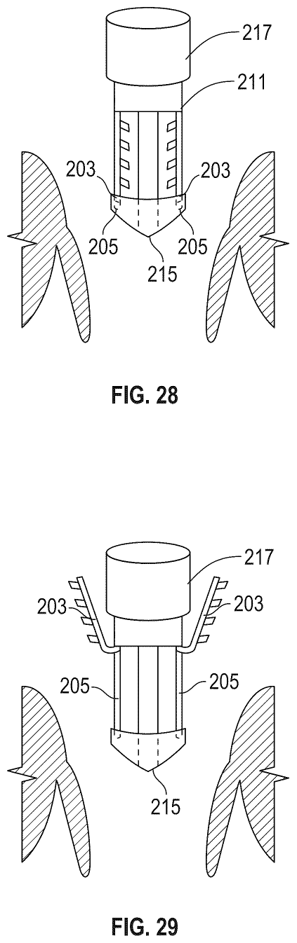

Referring to FIG. 28, in an undeployed configuration the second plurality of elongate anchor arms 205 may extend further than the first plurality of elongate anchor arms 203. As such, the retaining body 215 may be advanced distally relative to the arms 203, 205 to release the first plurality of elongate anchor arms 203 prior to release of the second plurality of elongate anchor arms 205. The second plurality of elongate anchor arms 205 may be deployed independently and sequentially with the first plurality of elongate anchor arms 203.

FIG. 29, for example, illustrates the retaining body 215 advanced distally relative to the arms 203, 205, with the first plurality of elongate anchor arms 203 moved to a deployed configuration similar to the configuration shown in FIGS. 8-11. The first plurality of elongate anchor arms 203 may be deployed in a similar manner as shown in FIGS. 8-11. The second plurality of elongate anchor arms 205 may be released by the retaining body 215 advancing distally, and the arms 205 may be deployed in a similar manner as shown in FIG. 26. In such an example, the use of a capsule 217 may be excluded, as another form of retaining body may retain the proximal end portion of the valve frame 211.

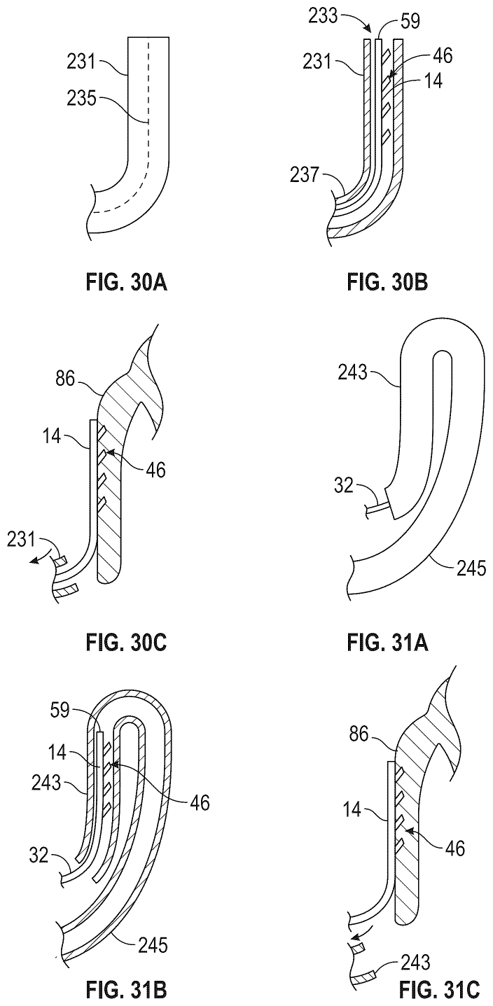

In examples, one or more covering bodies may be utilized to cover at least one of the one or more anchoring protrusions of the elongate anchor arms. FIGS. 30A-30C, for example, illustrate an example in which a covering body comprises a sheath 231 extending along one of the anchor arms 14 (shown in the cross sectional view of FIG. 30B). The sheath 231 may extend along the elongate anchor arm 14 to the tip 59 of the elongate anchor arm 14 and may have an opening 233 at the tip 59 of the elongate anchor 14 (as marked in FIG. 30B).

The sheath 231 may include a seam 235 extending along the length of the sheath 231 that may be configured to split or otherwise separate from the elongate anchor arm 14 to allow the sheath 231 to separate from the elongate anchor arm 14.

Each elongate anchor arm 14 may include a sheath 231 positioned thereon. A plurality of sheaths 231 may each extend along a respective one of the plurality of elongate anchor arms. The sheath 231 may be configured to cover the anchoring protrusions 46 to serve as a barrier layer or wall that reduces the possibility of contact between the anchoring protrusions 46 and the anchoring site for the anchoring protrusions 46. Thus, upon deployment, the sheath 231 may cover the anchoring protrusions 46 until a desired time at which the sheath 231 uncovers the anchoring protrusion 46 to allow the anchoring protrusions 46 to anchor.

FIG. 30B, for example, illustrates a cross sectional view of the anchor arm 14 within the sheath 231. A proximal portion 237 of the sheath 231 may be coupled to a retraction apparatus, such as gripping arms that may be part of the delivery apparatus. Upon delivery of the prosthetic implant, the retraction apparatus may retract the sheath 231 to expose the anchoring protrusions 46. FIG. 30C, for example, illustrates the sheath 231 having been retracted. The sheath 231 may open along the seam 235 to allow the retraction apparatus to remove the sheath 231 as desired, or the sheath 231 may remain in place proximal of the anchoring protrusions 46 as desired.

With the anchoring protrusions 46 exposed, the anchoring protrusions 46 may anchor to the heart valve leaflet as shown in FIG. 30C.

The movement of each sheath 231 may be independently controllable, to allow each sheath 231 to be retracted in a sequence as desired. Further, the sheath 231 may be advanced to cover the anchoring protrusions 46 as desired. In examples, the retraction apparatus may be configured to retract or advance each sheath 231 simultaneously or in a sequence as desired.

Variations in the configuration of the sheath may be provided in examples. FIGS. 31A-31C, for example, illustrate an example in which a sheath 243 is pulled from the elongate anchor arm 14 in a direction from the first end portion 32 of the anchor arm towards the distal tip 59 of the elongate anchor arm 14. The sheath 243, for example, may extend off from the distal tip 59 and may loop back radially inward to a retraction portion 245 of the sheath 243 that may be retracted to pull the sheath 243 off from the arm 14. FIG. 31B, for example, illustrates a cross sectional view of the sheath 243 showing the sheath 243 covering the anchoring protrusions 46. A retraction mechanism may pull the retraction portion 245 to uncover the anchoring protrusions 46.

FIG. 31C, for example, illustrates the sheath 243 having been removed from the elongate anchoring arm 14 to expose the anchoring protrusions 46 and to allow the anchoring protrusions 46 to anchor to the heart valve leaflet.

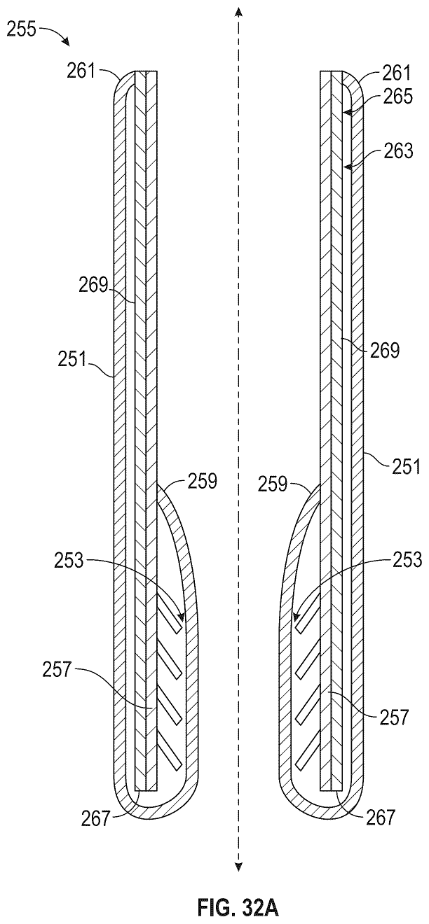

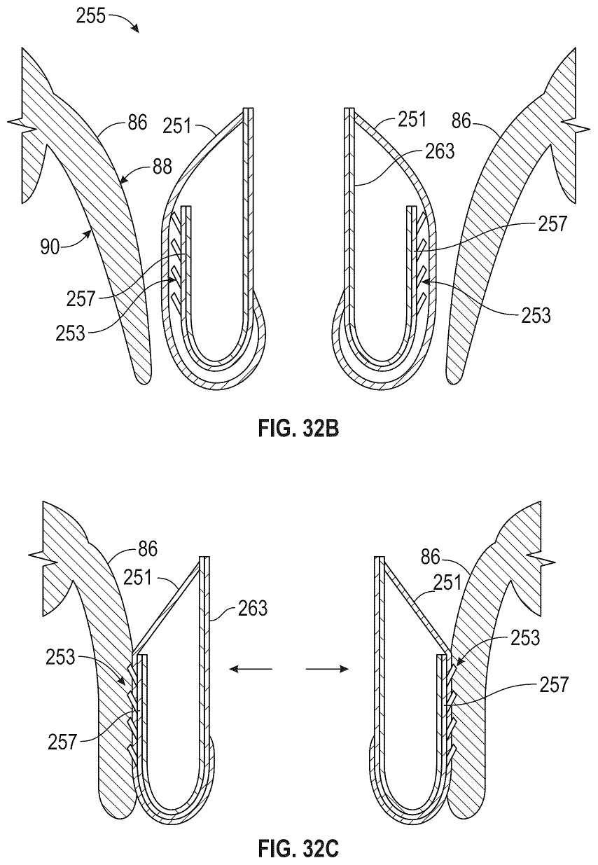

Other forms of covering bodies may be utilized in examples. FIGS. 32A-32C, for example, illustrates an example of a covering body comprising a skirt 251 that may cover the anchoring protrusions 253. FIG. 32A illustrates a prosthetic valve 255 that may be configured similarly as the prosthetic valve 10 shown in FIGS. 1-4, yet may include the skirt 251 coupled thereto. The prosthetic valve 255 is shown in an undeployed configuration with the elongate anchoring arms 257 being straightened and extending distally. The anchoring protrusions 253 may face radially inward as shown in FIG. 32A.

The skirt 251 may comprise a flexible skirt that the elongate anchoring arms 257 are positioned within. The skirt 251, for example, may comprise a container for the elongate anchoring arms 257 such as a pouch. The container may extend circumferentially about each of the elongate anchoring arms 257 and may cover each of the elongate anchoring arms 257. The skirt 251 may have a first end portion 259 that couples to an interior surface of the prosthetic valve 255 in an undeployed configuration and a second end portion 261 that may couple to an exterior surface of the prosthetic valve 255 in an undeployed configuration. The interior surface of the prosthetic valve 255, for example, may comprise an end portion of the elongate anchoring arms 257 or an interior surface of the valve body 263, among other positions. The exterior surface of the prosthetic valve 255 may comprise a proximal portion 265 of the valve body 263, among other positions. The skirt 251 may extend around a tip 267 of each of the elongate anchoring arms 257 in examples. A sealing skirt 269 may be coupled to the valve body 263 if desired, similar to the sealing skirt 18 discussed with respect to FIG. 3.

The skirt 251 may be stretchable and configured to be drawn taut as the prosthetic valve 255 moves to a deployed configuration. FIG. 32B, for example, illustrate the prosthetic valve 255 in a configuration with the elongate anchoring arms 257 rotated from the position shown in FIG. 32A. The elongate anchoring arms 257 may rotate within the skirt 251 to move from the undeployed configuration to the deployed configuration shown in FIG. 32B. In the configuration shown in FIG. 32B, the valve body 263 may not yet be expanded fully radially outward. As such, the skirt 251 may remain loose and configured to allow the elongate anchoring arms 257 to rotate therein. Further, the skirt 251 may cover the anchoring protrusions 253 and reduce the possibility of anchoring of the anchoring protrusions 253 with the interior surfaces 88 of the native heart valve leaflets at this point. The prosthetic valve 255 may be slid proximally or distally within the heart valve annulus for a desired positioning, without the anchoring protrusions 253 yet anchoring to the native heart valve leaflets.

At a desired time, the valve body 263 may be expanded radially outward as shown in FIG. 32C, which may cause the anchoring arms 257 to press against the skirt 251 and allow the anchoring protrusions 253 to pass through the skirt 251. The anchoring protrusions 253 may pass through the skirt 251 to penetrate and anchor to the heart valve leaflets. The expansion of the valve body 263 may cause the skirt 251 to be pulled taut, which may aid the penetration of the skirt 251 by the anchoring protrusions 253. The skirt 251 may remain in position to provide a seal with the heart valve. The anchoring protrusions 253 may puncture the skirt 251 or in examples the skirt 251 may include openings for the anchoring protrusions 253 to pass through.

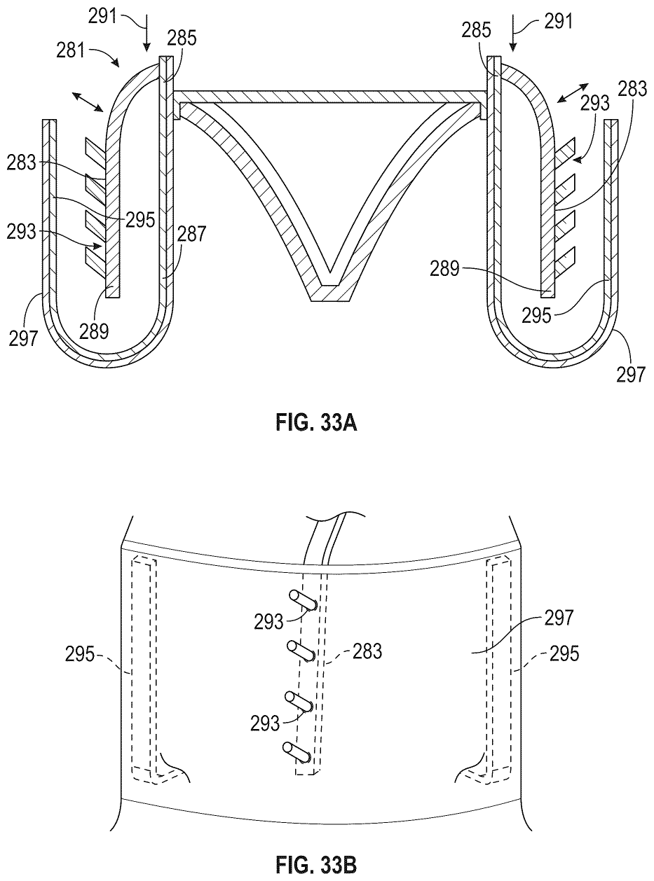

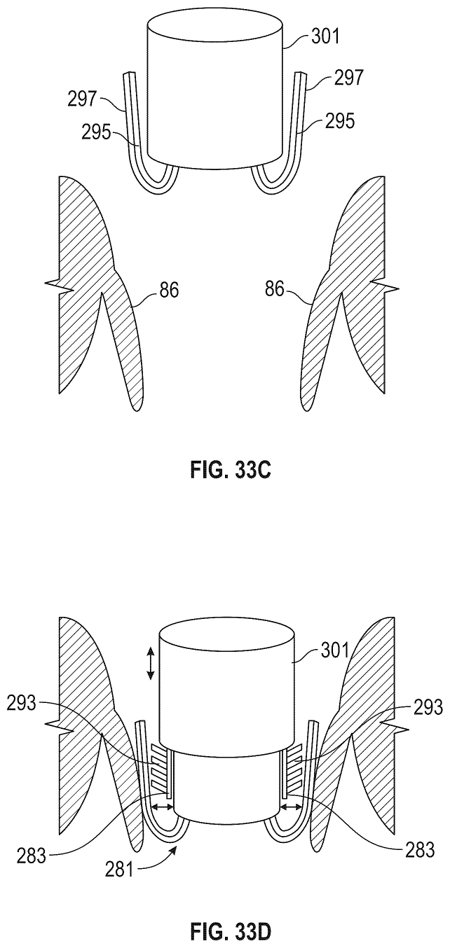

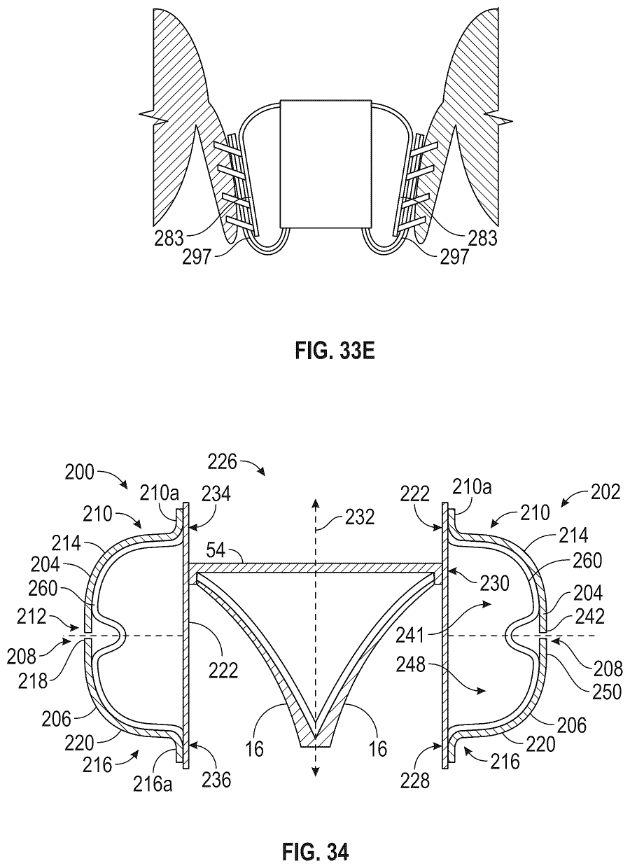

FIGS. 33A-33E illustrate an example of a prosthetic valve 281 in which the elongate anchoring arms 283 at a first end portion are each coupled to a proximal end portion 285 of the valve frame 287 and extend distally to a second end portion or distal end 289 of the respective elongate anchoring arm 283. Each elongate anchoring arm 283 may be configured to deflect radially inward and outward upon a force being applied to the respective elongate anchoring arm 283. For example, a force applied distally (in a direction indicated by arrows 291) or radially inward to the elongate anchoring arms 283 may cause the arms 283 to move radially inward. A removal of the force may allow the elongate anchoring arms 283 to move radially outward. For example, the elongate anchoring arms 283 may be biased to deflect radially outward and a force applied by a delivery apparatus may deflect the elongate anchoring arms 283 radially inward. A capsule of a delivery apparatus, for example, may apply the force to move the elongate anchoring arms 283 radially inward. In such a manner, a radial position of the elongate anchoring arms 283 may be controlled by a force applied to the arms 283.

Each of the second end portions of the arms 283 may include anchoring protrusions 293 that may be configured similarly as the anchoring protrusions 46 shown in FIGS. 1-4. The anchoring protrusions 293 may be configured to anchor to an interior surface of heart valve leaflets upon contacting the interior surface of the heart valve leaflet.

A plurality of arms 295 may extend radially outward of the valve frame 287 and may be configured to be positioned radially outward of the elongate anchoring arms 283 when the elongate anchoring arms 283 are deflected radially inward. Each of the plurality of arms 295 may be configured to hook in a similar manner as the elongate anchor arms 14 shown in FIGS. 1-4. The arms 295 may form a ring extending around the valve frame 287. The arms 295 may support a skirt 297 that may form a ring around the valve frame 287. The skirt 297 may extend circumferentially about the arms 295, the second end portions of the elongate anchoring arms 283, and the valve frame 287.

FIG. 33B, for example, illustrates a perspective view of the skirt 297 supported by the arms 295. The arms 295 may be circumferentially spaced from each other and may extend radially outward from the valve frame 287 in a similar manner as the elongate anchor arms 14 shown in FIGS. 1-4. The arms 295, however, may lack anchoring protrusions in examples. The elongate anchoring arms 283 may be positioned between the arms 295 and may include anchoring protrusions 293 that are configured to pass through the skirt 297 to anchor to a surface. The skirt 297, for example, may include openings that the anchoring protrusions 293 may pass through, or the anchoring protrusions 293 may puncture through the skirt 297 to anchor to a surface.

A distance between the anchoring protrusions 293 and the skirt 297, however, may be controlled by the force applied to the elongate anchoring arms 283 as discussed with respect to FIG. 33A. With a distal or radially inward force applied to the elongate anchoring arms 283, the anchoring protrusions 293 may be retracted radially inward from the skirt 297. With the distal force removed from the elongate anchoring arms 283, the elongate anchoring arms 283 may extend radially outward to pass through the skirt 297. As such, during deployment, a user may determine the point during the deployment sequence that the anchoring protrusions 293 pass through the skirt 297 to anchor to a surface by controlling the force applied to the elongate anchoring arms 283. Prior to such a point, the prosthetic valve 281 may be slid proximally and distally within the heart valve annulus without the anchoring protrusions 293 anchoring to the surface. Further, upon anchoring of the anchoring protrusions 293 to the surface through the skirt 297, the anchoring protrusions 293 may be retracted by a distal or radially inward force applied to the arms 283 to allow for disengagement of the protrusions 293 with the surface. A recapture or repositioning of the prosthetic valve 281 may occur.

FIGS. 33C-33E, for example, illustrate an exemplary deployment sequence of the prosthetic valve 281. In FIG. 33C, the elongate arms 295 may be deployed in a similar manner as the elongate anchoring arms 14 discussed with respect to FIGS. 1-4. The arms 295 may support the skirt 297. The arms 295 and skirt 297 may be slid freely with respect to the heart valve leaflets 86 due to the lack of anchoring protrusions. A capsule 301 or other retaining body may apply a force to retain the elongate anchoring arms 283 radially inward.

Referring to FIG. 33D, the elongate arms 295 and skirt 297 may be slid within the native heart valve annulus until placed in a desired position. The capsule 301 may then be retracted to remove the force applied to the elongate anchoring arms 283. The elongate anchoring arms 283 may be able to deflect radially outward to pass through the skirt 297 and anchor to the interior surface of the leaflets. If recapture or repositioning of the prosthetic valve 281 is desired, the capsule 301 may be advanced relative to the elongate anchoring arms 283 to retract the arms 283 and the anchoring protrusion 293 radially inward.

FIG. 33E illustrates the elongate anchoring arms 283 fully deployed and passing through the skirt 297 to anchor to the interior surface of the heart valve.

Other configurations of prosthetic valves may be utilized in examples.

FIG. 34 illustrates a schematic cross sectional view of a prosthetic valve 200. The prosthetic valve 200 may include a plurality of prosthetic valve leaflets 16 and may include an anchoring body 202 coupled to the plurality of prosthetic valve leaflets 16. The anchoring body 202 may be configured to anchor to a heart valve.

The anchoring body 202 may include a proximal frame 204 and a distal frame 206. The anchoring body 202 may be configured to form an annular recess 208 between the proximal frame 204 and the distal frame 206 for receiving a portion of the heart valve.

The proximal frame 204 may include a proximal end portion 210 and a distal end portion 212. The proximal frame 204 may extend radially outward from the proximal end portion 210 to form a first shoulder 214 that is positioned proximal of the annular recess 208. The distal frame 206 may include a distal end portion 216 and a proximal end portion 218. The distal frame 206 may extend radially outward from the distal end portion 216 to form a second shoulder 220 that is positioned distal of the annular recess 208.

In examples, an inner frame 222 may be utilized with the prosthetic valve 200 that may support the plurality of prosthetic valve leaflets 16. The inner frame 222, for example, may have a configuration similar to the frame 224 shown in FIG. 45 and may surround a central channel 226 for fluid flow therethrough. The inner frame 222 may include a plurality of struts spaced by openings, which may be a similar configuration as the frame 224 shown in FIG. 45. The inner frame 222 may have a cylindrical shape in examples, similar to the frame 224 shown in FIG. 45, or may have another shape as desired.

The inner frame 222 may have an interior surface 228 that faces inward towards the central channel 226 and an outer surface 230 that faces opposite the interior surface 228. The prosthetic valve leaflets 16 may extend radially inward from the interior surface 228 of the inner frame 222 towards a central axis 232 and may be supported by a support body 54. The inner frame 222 may have a proximal end portion 234 and extend along the central axis 232 to a distal end portion 236.

The proximal frame 204 may include a plurality of struts 238 separated by openings 240 as shown in FIG. 35 for example. In examples, the proximal end portion 210 of the proximal frame 204 may be coupled to the inner frame 222 and may include a coupling portion 210a extending axially that may extend parallel with the inner frame 222. The proximal frame 204 may extend radially outward from the proximal end portion 210 to form the shoulder 214. The shoulder 214 may be positioned with a space 241 between the shoulder 214 and the inner frame 222 in examples.

The proximal frame 204 may extend distally from the shoulder 214 to the distal end portion 212. As shown in FIG. 34, the proximal frame 204 may extend axially from the shoulder 214 to the distal end portion 212, which may be parallel with the inner frame 222 in examples. The proximal frame 204 may extend distally to a distal end 242 of the proximal frame 204, which may be spaced from the inner frame 222 in examples.

The distal frame 206 may include a plurality of struts 244 separated by openings 246 as shown in FIG. 35 for example. In examples, the distal end portion 216 of the distal frame 206 may be coupled to the inner frame 222 and may include a coupling portion 216a extending axially that may extend parallel with the inner frame 222. The distal frame 206 may extend radially outward from the distal end portion 216 to form the shoulder 220. The shoulder 220 may be positioned with a space 248 between the shoulder 220 and the inner frame 222 in examples.

The distal frame 206 may extend proximally from the shoulder 220 to the proximal end portion 218. As shown in FIG. 34, the distal frame 206 may extend axially from the shoulder 220 to the proximal end portion 218, which may be parallel with the inner frame 222 in examples. The distal frame 206 may extend proximally to a proximal end 250 of the distal frame 206, which may be spaced from the inner frame 222 in examples.

The proximal end 250 of the distal frame 206 may be spaced from the distal end 242 of the proximal frame 204 with a gap between the ends 250, 242 as shown in FIG. 34. In examples, the gap may comprise the recess for receiving a portion of the heart valve. In examples, the distal end portion 212 of the proximal frame 204 and the proximal end portion 218 of the distal frame 206 may be angled radially inward to form the recess for receiving a portion of the heart valve.

FIG. 35, for example, illustrates a configuration of the proximal frame 204 and the distal frame 206 in which the distal end portion 212 of the proximal frame 204 and the proximal end portion 218 of the distal frame 206 may be angled radially inward to form the recess 208 for receiving a portion of the heart valve. In examples, the distal end portion 212 of the proximal frame 204 and the proximal end portion 218 of the distal frame 206 may each extend parallel with the inner frame 222 as shown in FIG. 34 for example.

In examples, the proximal frame 204 may be flexible and may be deflectable radially inward, and the distal frame 206 may be flexible and may be deflectable radially inward. The distal end 242 of the proximal frame 204 for example, and the proximal end 250 of the distal frame 206 for example, may be unsupported directly by the inner frame 222 and may be able to deflect in a direction radially inward. In examples, the portion of the proximal frame 204 distal of the shoulder 214, and the portion of the distal frame 206 proximal of the shoulder 220 may be deflectable radially inward. In examples, the proximal end portion 210 and the shoulder 214 of the proximal frame 204, and the distal end portion 216 and the shoulder 220 of the distal frame 206 may each maintain their shape upon the portion of the proximal frame 204 and the portion of the distal frame 206 deflecting radially inward. In examples, the proximal frame 204 may continue to extend radially outward from the proximal end portion 210 to form the first shoulder 214 positioned proximal of the annular recess 208, and the distal frame 206 may continue to extend radially outward from the distal end portion 216 to form the second shoulder 220 positioned distal of the annular recess 208.

In examples, the portion of the proximal frame 204 distal of the shoulder 214, and the portion of the distal frame 206 proximal of the shoulder 220 may be deflectable radially inward to increase the horizontal depth of the annular recess 208 and to allow the heart valve to extend further radially inward relative to the anchoring body 202. The shoulders 214, 220 accordingly may protrude radially outward relative to the annular recess 208 and may serve to stabilize the anchoring body 202 relative to the portion of the heart valve within the annular recess 208. The presence of the proximal frame 204 and the shoulder 214 may resist distal movement of the anchoring body 202 upon the anchoring body 202 having been deployed, and the presence of the distal frame 206 and the shoulder 220 may resist proximal movement of the anchoring body 202 upon the anchoring body 202 having been deployed.

In examples, the proximal frame 204 and the distal frame 206 may clamp the portion of the heart valve positioned within the annular recess 208 between the proximal frame 204 and the distal frame 206. Movement of the anchoring body 202 relative to the heart valve in an axial direction accordingly may be reduced upon deployment.

Referring to FIG. 34, in examples, a sealing skirt 260 may extend along one or more of the proximal frame 204 or the distal frame 206. The sealing skirt 260 may extend on an outer surface of the proximal frame 204 or the distal frame 206, or on an inner surface of the proximal frame 204 or the distal frame 206, or combinations thereof. In examples, the sealing skirt 260 may extend from the proximal end portion 210 of the proximal frame 204 to the distal end portion 216 of the distal frame 206. The sealing skirt 260 may be configured to be positioned within the annular recess 208 in examples, to provide for fluid seal at the annular recess 208. The sealing skirt 260 may provide a fluid seal to reduce fluid flow outside of the central channel 226.

FIG. 35 illustrates a side schematic view of a configuration of the proximal frame 204 and the distal frame 206 upon the inner frame 222. The proximal frame 204 and the distal frame 206 may each include a plurality of struts 238, 244 separated by respective openings 240, 246. In an example in which the proximal frame 204 and distal frame 206 may be deflectable radially inward, the configuration of struts 238, 244 may allow for flexibility and deflection of the respective proximal frame 204 and distal frame 206. Certain features of the prosthetic valve 200 such as the sealing skirt 260, the prosthetic valve leaflets 16, and the struts of the inner frame 222 are not shown in FIG. 35 for clarity.

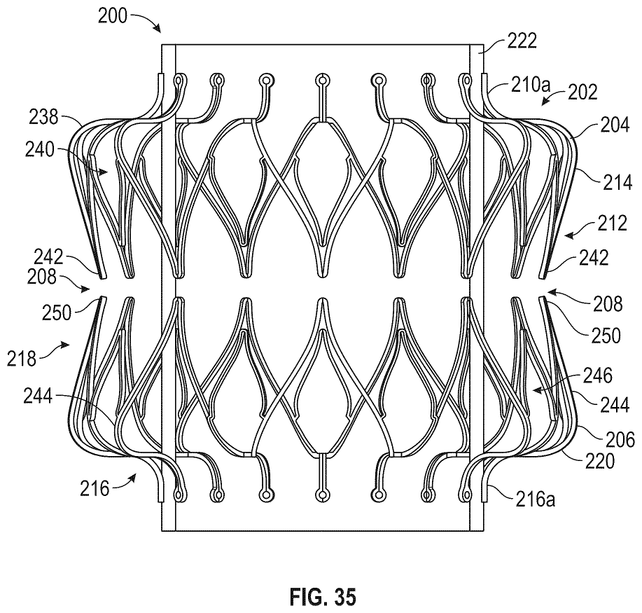

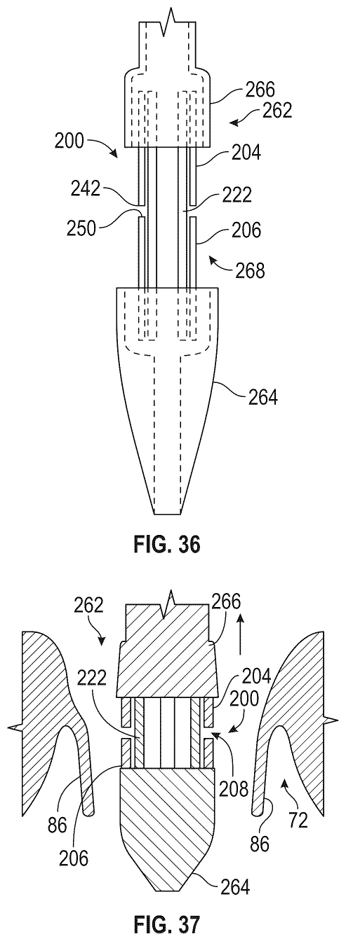

In examples, the prosthetic valve 200 may be configured to move from an undeployed configuration to a deployed configuration. The proximal frame 204, distal frame 206, and the inner frame 222 may each accordingly be configured to move from the undeployed configuration to the deployed configuration. In examples, the frames 204, 206, 222 may be radially compressed in the undeployed configuration from the configurations shown in FIGS. 34 and 35 and may be elongated. The shoulders 214, 220 of the respective proximal frame 204 and distal frame 206 may further be radially compressed. The prosthetic valve 200 may have an elongate cylindrical shape in the undeployed configuration as shown in FIG. 36 for example. The prosthetic valve 200 may be configured to expand to a deployed configuration as shown in FIGS. 34 and 35, in which the length of the prosthetic valve 200 reduces and the diameter of the prosthetic valve 200 increases.

FIG. 36 illustrates the prosthetic valve 200 in an undeployed configuration and positioned within a capsule 262. The reduced diameter of the prosthetic valve 200 may allow for enhanced transvascular and transcatheter entry into the patient's body. The capsule 262 may comprise a capsule of a delivery apparatus 62 as shown in FIG. 5, for example. The capsule 262 may surround an implant retention area for the prosthetic valve 200.

In examples, the capsule 262 may comprise a split capsule that may be configured to have multiple portions release from the prosthetic valve 200 upon deployment. The capsule 262 for example, may include a distal portion 264 and a proximal portion 266 that may be configured to release from the prosthetic valve 200. The distal portion 264 and the proximal portion 266 may each be configured to release from the prosthetic valve 200 separately. The release of the proximal portion 266 may be independent of the release of the distal portion 264 in examples.

The proximal portion 266 of the capsule 262 may be positioned at the proximal frame 204 and the distal portion 264 of the capsule 262 may be positioned at the distal frame 206. The proximal portion 266 and the distal portion 264 may be separate from each other such that a central portion 268 of the capsule 262 leaves a central portion of the prosthetic valve 200 uncovered. The central portion of the prosthetic valve 200 may remain uncovered by the capsule 262 in examples to enhance flexibility of the delivery apparatus and capsule 262 upon passing through the patient's vasculature. In examples, the central portion of the prosthetic valve 200 may be covered by the capsule 262.

The proximal portion 266 of the capsule 262 may operate independently from the distal portion 264 of the capsule 262 in examples and may be configured to release the proximal frame 204 independent from the distal frame 206. Similarly, the distal portion 264 of the capsule 262 may be configured to release the distal frame 206 independent from the proximal frame 204. Such a feature may allow for multi-stage deployment of the prosthetic valve 200 with each frame 204, 206 deployed in a desired sequence. In examples, each portion 266, 264 of the capsule 262 may be configured to simultaneously release the respective portions 264, 266 of the capsule 262. In examples, a split capsule may not be utilized and rather a single body capsule may retract from the prosthetic valve 200 to deploy the prosthetic valve 200 in examples.

FIGS. 37-39 illustrate an exemplary deployment sequence of the prosthetic valve 200. The capsule 262 may be utilized in the deployment sequence. FIG. 37 illustrates the prosthetic valve 200 in the undeployed configuration positioned within the capsule 262 as shown in FIG. 36 for example. The prosthetic valve 200 may be advanced to the desired implantation site in the undeployed configuration.

The prosthetic valve 200 may be aligned axially with respect to the desired implantation site, which may be a heart valve 72. The prosthetic valve 200 may be positioned within the heart valve 72 and may be positioned between the heart valve leaflets 86. The capsule 262 may be advanced or retracted to place the prosthetic valve 200 in the desired orientation relative to the heart valve 72. The position of the capsule 262 and the prosthetic valve 200 may be visualized utilizing medical imaging, which may utilize techniques disclosed herein.

In examples, the prosthetic valve 200 may be positioned such that the annular recess 208 is aligned with the annulus of the heart valve 72. In examples, the prosthetic valve 200 may be positioned with the annular recess 208 aligned with other portions of the heart valve 72 such as the leaflets 86. In examples, the prosthetic valve 200 may be aligned with a combination of the annulus and the leaflets 86.

With the prosthetic valve 200 in a desired position relative to the heart valve 72, one or more of the portions 266, 264 of the capsule 262 may release from the prosthetic valve 200. For example, as shown in FIG. 37, the proximal portion 266 of the capsule 262 may retract proximally to release the proximal frame 204 and allow the proximal frame 204 to expand radially outward.

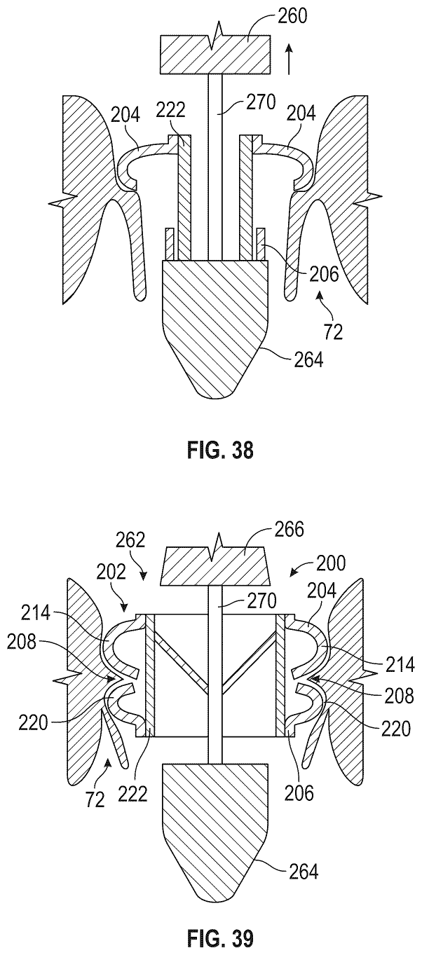

FIG. 38, for example, illustrates the proximal portion 266 of the capsule 262 having retracted proximally and the proximal frame 204 having expanded radially outward. The distal frame 206 may remain compressed and within the distal portion 264 of the capsule 262.

In the configuration shown in FIG. 38, the position of the proximal frame 204 relative to the heart valve 72 may be imaged to determine if the desired alignment between the proximal frame 204 and the heart valve 72 results. In examples, the prosthetic valve 200 may be repositioned with the distal frame 206 remaining in an undeployed configuration within the distal portion 264 of the capsule 262. For example, if a user (e.g., a clinician) determines that the proximal frame 204 is positioned undesirably proximal (e.g., too far atrial), then the prosthetic valve 200 may be repositioned distal. If a user (e.g., a clinician) determines that the proximal frame 204 is positioned undesirably distal (e.g., too far ventricular), then the prosthetic valve 200 may be repositioned proximal.

With the proximal frame 204 in the desired position, the distal frame 206 may be released from the capsule 262. The distal portion 264 of the capsule 262 for example, may be advanced distally to release the distal frame 206.

FIG. 39 illustrates the proximal frame 204 and the distal frame 206 having been released from the capsule 262. A portion of the heart valve 72 is received by the annular recess 208. The proximal frame 204 is positioned proximal of the portion of the heart valve 72 within the annular recess 208 and the distal frame 206 is positioned distal of the portion of the heart valve 72 within the annular recess 208. The anchoring body 202 anchors to the interior surface of the heart valve 72.

The shoulder 214 of the proximal frame 204 protrudes radially outwardly relative to the annular recess 208 to impede distal movement of the prosthetic valve 200 upon being deployed. The shoulder 220 of the distal frame 206 protrudes radially outwardly relative to the annular recess 208 to impede proximal movement of the prosthetic valve 200 upon being deployed.

As shown in FIG. 39, the distal end of the proximal frame 204 and the proximal end of the distal frame 206 may deflect radially inward in examples to increase the horizontal depth of the annular recess 208. Accordingly, a greater amount of the heart valve 72 may be positioned within the annular recess 208 due to the inward deflection of the proximal frame 204 and the distal frame 206.

The proximal frame 204 and the distal frame 206 may be flexible and resilient in an axial direction in examples, to resist axial movement of the prosthetic valve 200. For example, upon a proximal axial force being applied to the prosthetic valve 200, the distal frame 206 may deflect axially to resist the proximal axial force and absorb the proximal axial force. The distal frame 206 may apply a resilient force to maintain the position of the prosthetic valve 200 within the heart valve 72. In a similar manner, upon a distal axial force being applied to the prosthetic valve 200, the proximal frame 204 may deflect axially to resist the distal axial force and absorb the distal axial force. The proximal frame 204 may apply a resilient force to maintain the position of the prosthetic valve 200 within the heart valve 72. The distal frame 206 and proximal frame 204 may operate to maintain the position of the prosthetic valve 200 within the heart valve 72.

The capsule 262 may be withdrawn from the deployed prosthetic valve 200, with the prosthetic valve 200 remaining in position within the heart valve 72. For example, an interior shaft 270 may extend between the distal portion 264 and the proximal portion 266 of the capsule 262 and may be withdrawn proximally to retract the distal portion 264 from the deployed prosthetic valve 200.

In examples, the deployment procedure may be varied from the sequence shown in FIGS. 37-39. For example, the distal portion 264 of the capsule 262 may be released prior to the proximal portion 266 of the capsule 262 being released. In examples, a single body capsule may be utilized to deploy the prosthetic valve 200. Further, in examples, the proximal frame 204 or the distal frame 206 may be configured to not deflect radially inward or deflect axially. In such a configuration, the heart valve 72 may continue to be positioned within the annular recess 208 formed by the proximal frame 204 and the distal frame 206.

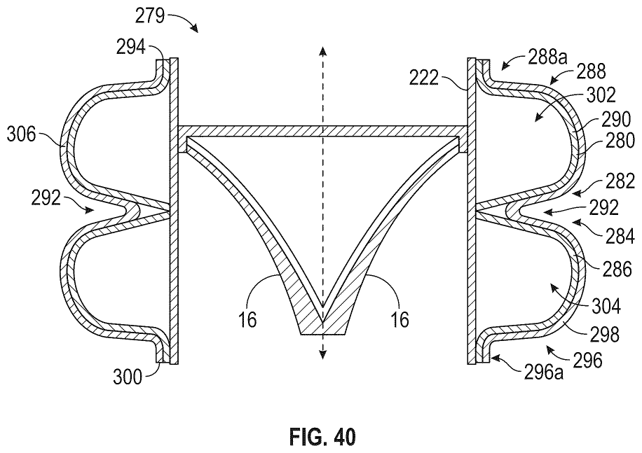

FIG. 40 illustrates an example of a prosthetic valve 279 in which the proximal frame 280 has a distal end portion 282 that is coupled to a proximal end portion 284 of the distal frame 286. In examples, the distal end portion 282 of the proximal frame 280 may be coupled to the inner frame 222, and the proximal end portion 284 of the distal frame 286 may be coupled to the inner frame 222. FIG. 41, for example, illustrates a side schematic view of the distal end portion 282 of the proximal frame 280 coupled to the inner frame 222 and the proximal end portion 284 of the distal frame 286. FIG. 41 illustrates the proximal end portion 284 of the distal frame 286 coupled to the inner frame 222 and the distal end portion 282 of the proximal frame 280.

In the example shown in FIGS. 40 and 41, the proximal end portion 288 of the proximal frame 280 may be configured similarly as with an example shown in FIG. 34 for example. The proximal frame 280 may extend radially outward from the proximal end portion 288 to form a first shoulder 290 positioned proximal of the annular recess 292. The proximal frame 280 may include a coupling portion 288a that may have a proximal end 294 that extends axially.

The distal end portion 296 of the distal frame 286 may be configured similarly as with an example shown in FIG. 34 for example. The distal frame 286 may extend radially outward from the distal end portion 296 to form a second shoulder 298 positioned distal of the annular recess 292. The distal frame 286 may include a coupling portion 296a that may have a distal end 300 that extends axially.

A space 302 may be positioned between the inner frame 222 and the proximal frame 280. The space 302 may have an annular shape and may be positioned proximal of the annular recess 292. A space 304 may be positioned between the inner frame 222 and the distal frame 286. The space 304 may have an annular shape and may be positioned distal of the annular recess 292.

The coupling of the proximal frame 280 to the distal frame 286 may increase the stability of the frames 280, 286 relative to each other. Further, in an example in which the distal end portion 282 of the proximal frame 280 and the proximal end portion 284 of the distal frame 286 are coupled to the inner frame 222, the horizontal depth of the annular recess 292 may be increased. The stability of the proximal frame 280 and the distal frame 286 relative to the inner frame 222 may be increased.

In examples, a sealing skirt 306 may be utilized that may extend along one or more of the proximal frame 280 or the distal frame 286, and may be positioned within the annular recess 292 as shown in FIG. 40.

In examples, one or more of the proximal frame 280 or the distal frame 286 may be axially compressible, as discussed with respect to FIG. 39. An axial compression may absorb an axial force applied to the prosthetic valve 279.

The prosthetic valve 279 may be deployed in a similar manner as the prosthetic valve 200. The proximal frame 280 may be deployed independently of the distal frame 286 to a desired implantation site. A portion of a heart valve may be received by the annular recess 292.

FIG. 41 illustrates a side schematic view of the proximal frame 280 and distal frame 286 positioned upon the inner frame 222. In examples, the distal end portion 282 of the proximal frame 280 may be coupled directly to the proximal end portion 284 of the distal frame 286, or one or more coupling bodies 307 may be utilized as desired. The coupling bodies 307 may be utilized to couple the distal end portion 282 of the proximal frame 280 or the proximal end portion 284 of the distal frame 286 to the inner frame 222 in examples as desired.

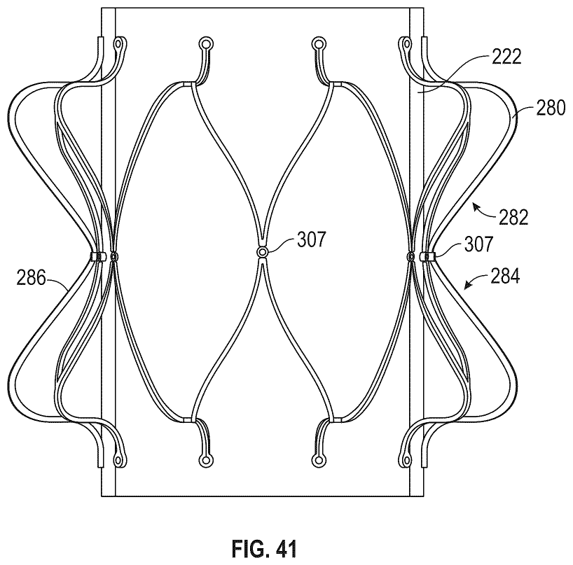

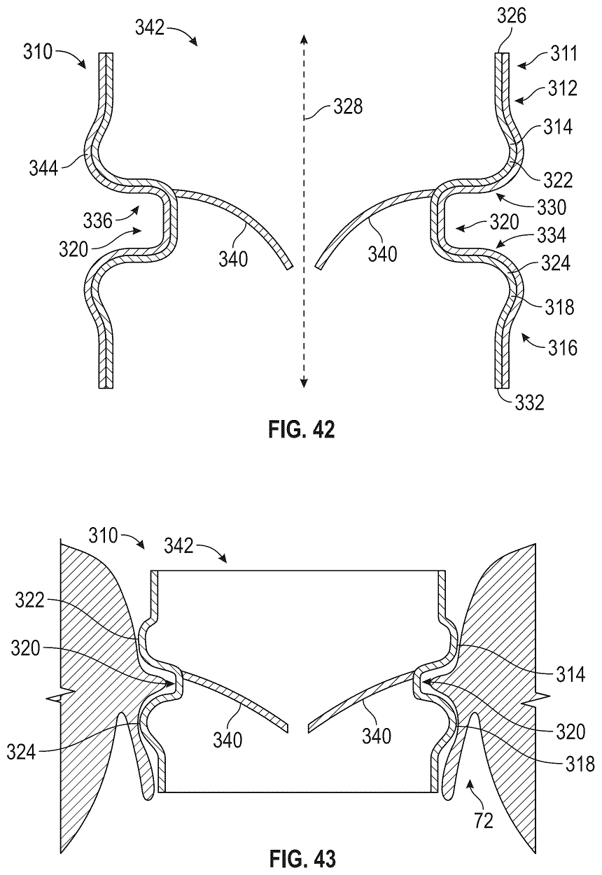

FIG. 42 illustrates an example of a prosthetic valve 310 in which the anchoring body 311 includes a proximal frame 314 and a distal frame 318. The anchoring body 311 may be configured to form an annular recess 320 between the proximal frame 314 and the distal frame 318 for receiving a portion of a heart valve. The proximal end portion 312 of the proximal frame 314 may be uncoupled to an inner frame and extends axially. The distal end portion 316 of the distal frame 318 may be uncoupled to an inner frame and extends axially.

The proximal frame 314 may extend radially outward from the proximal end portion 312 to form a first shoulder 322 positioned proximal of the annular recess 320. The distal frame 318 may extend radially outward from the distal end portion 316 to form a second shoulder 324 positioned distal of the annular recess 320.

The proximal frame 314 may have a proximal end 326 that extends axially and may extend parallel with the central axis 328 of the prosthetic valve 310. The proximal end 326 may form an annular opening of the proximal side of the prosthetic valve 310 and may have a cylindrical shape. The first shoulder 322 may extend radially outward from the proximal end 326. The distal end portion 330 of the proximal frame 314 may extend radially inward from the first shoulder 322 and may form a portion of the annular recess 320.

The distal frame 318 may have a distal end 332 that extends axially and may extend parallel with the central axis 328 of the prosthetic valve 310. The distal end 332 may form an annular opening of the distal side of the prosthetic valve 310 and may have a cylindrical shape. The second shoulder 324 may extend radially outward from the distal end 332. The proximal end portion 334 of the distal frame 318 may extend radially inward from the second shoulder 324 and may form a portion of the annular recess 320.

The distal end portion 330 of the proximal frame 314 and the proximal end portion 334 of the distal frame 318 may be coupled together at the annular recess 320 and may form a central portion 336 of the anchoring body 311. The central portion 336 may have a cylindrical shape in examples and may be positioned radially inward of the proximal end 326 and the distal end 332. In examples, the annular recess 320 may be shaped as a rectangular annulus about the anchoring body 311 due to the shape of the transition between the central portion 336 and the first shoulder 322 and the second shoulder 324. In examples, the annular recess 320 may have other shapes.

In examples, the anchoring body 311 may support the plurality of leaflets 340 within the central channel 342. For example, the plurality of leaflets 340 may extend radially inward from the anchoring body 311 and in examples may be coupled directly to the anchoring body 311. The plurality of leaflets 340 may be coupled to the central portion 336 of the anchoring body 311 in examples, or another position as desired.

In examples, a sealing skirt 344 may be provided that may extend along one or more of the proximal frame 314 or the distal frame 318. The sealing skirt 344 as shown in FIG. 42 for example, may be positioned on an outer surface of the anchoring body 311. In examples, the scaling skirt 344 may be positioned on an inner surface of the anchoring body 311.

The prosthetic valve 310 may be deployed in a similar manner as the prosthetic valve 200, and as represented in FIGS. 37-39. For example, a multi-stage deployment of the prosthetic valve 310 may occur, with the proximal frame 314 deployed independently from the distal frame 318. In examples, a single body capsule may be utilized to deploy the prosthetic valve 310. Other forms of deployment may be utilized in examples.

FIG. 43 illustrates the prosthetic valve 310 deployed to a heart valve 72. A portion of the heart valve 72 may be positioned within the annular recess 320. The proximal frame 314 and distal frame 318 may clamp the heart valve 72 within the annular recess 320. The proximal frame 314 and the distal frame 318 may provide a similar function as the proximal frame 204 and distal frame 206 shown in FIG. 39. The proximal frame 314 and distal frame 318 may axially stabilize the prosthetic valve 310 within the heart valve 72, with the shoulders 322, 324 protruding radially outward from the annular recess 320.

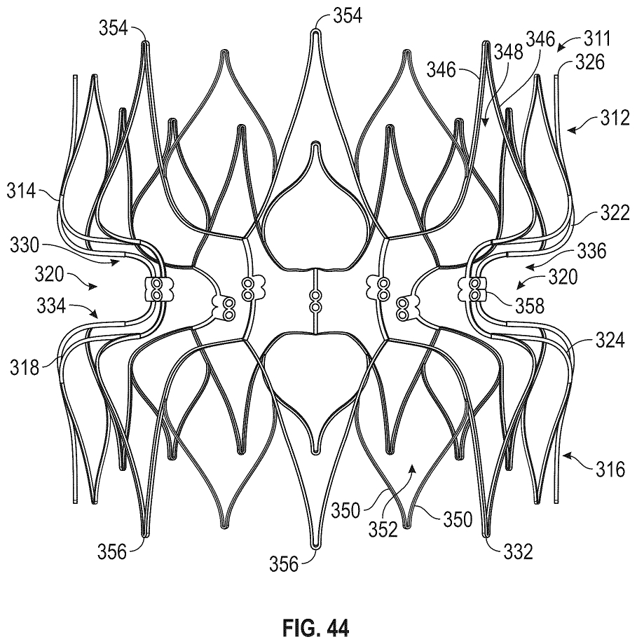

FIG. 44 illustrates a perspective view of the anchoring body 311. The proximal frame 314 may include a plurality of struts 346 separated by openings 348. The distal frame 318 may similarly include a plurality of struts 350 separated by openings 352. The proximal frame 314 may be configured such that the proximal end 326 may include apices 354 of struts 346 that may extend proximally. The distal frame 318 may be configured such that the distal end 332 may include apices 356 of struts 346 that may extend distally.

In examples, coupling bodies 358 may couple the distal end portion 330 of the proximal frame 314 to the proximal end portion 334 of the distal frame 318. In examples, the proximal frame 314 may be coupled directly to the distal frame 318. The configuration of the anchoring body 311 shown in FIG. 44 may be varied in examples.

In examples, an inner frame 224 may be utilized with the anchoring body 311. FIG. 45, for example, illustrates a perspective view of an inner frame 224 that may be utilized according to examples herein. The inner frame 224 may include a plurality of struts 360 separated by openings 362. The inner frame 224 may have a cylindrical shape in examples, or may have another shape as desired.

In examples, the inner frame 224 may include coupling bodies 364 that may be configured to couple to the anchoring body 311. The coupling bodies 364 may couple to the coupling bodies 358 of the anchoring body 311. In examples, the coupling bodies 364 may be coupled to the prosthetic valve leaflets 340 shown in FIGS. 42 and 43 for example. The inner frame 224 may support the prosthetic valve leaflets 340 within the central channel of the inner frame 224.

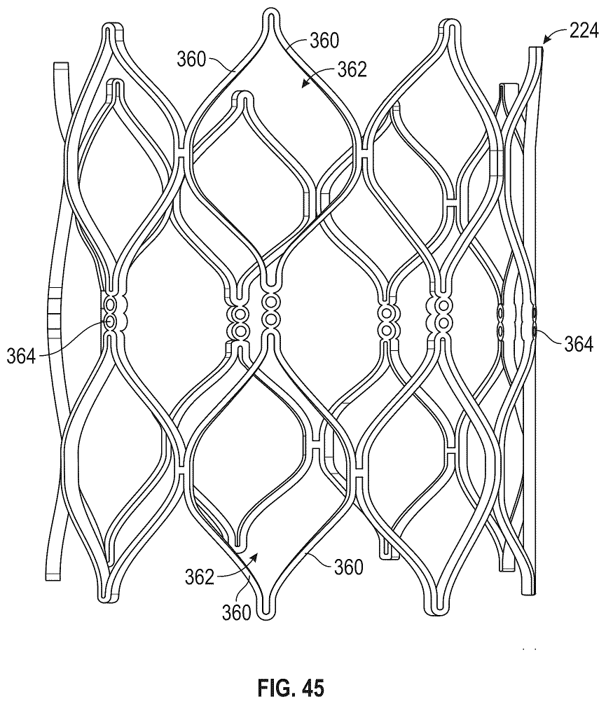

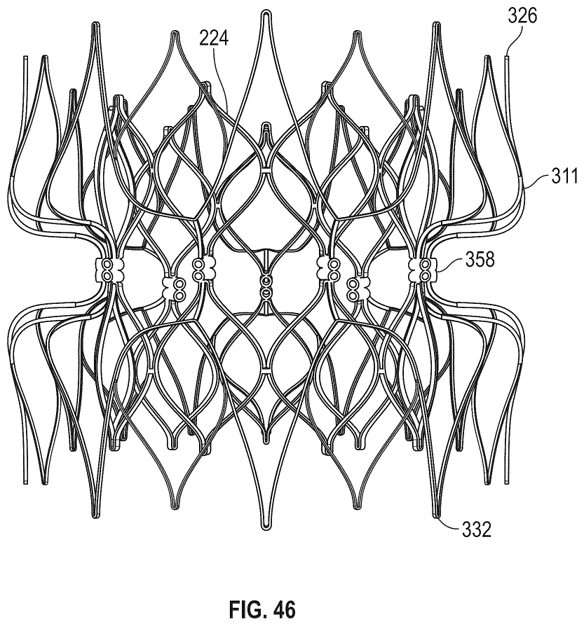

FIG. 46 illustrates a perspective view of the inner frame 224 positioned within the anchoring body 311. The anchoring body 311 is positioned radially outward of the inner frame 224. The distal end 332 of the anchoring body 311 may be spaced from the inner frame 224 and the proximal end 326 of the anchoring body 311 may be spaced from the inner frame 224. The coupling body 358 of the anchoring body 311 may be coupled to the coupling body 364 of the inner frame 224.

The features of the examples of FIGS. 34-46 may be utilized solely or in combination with any other example disclosed herein.

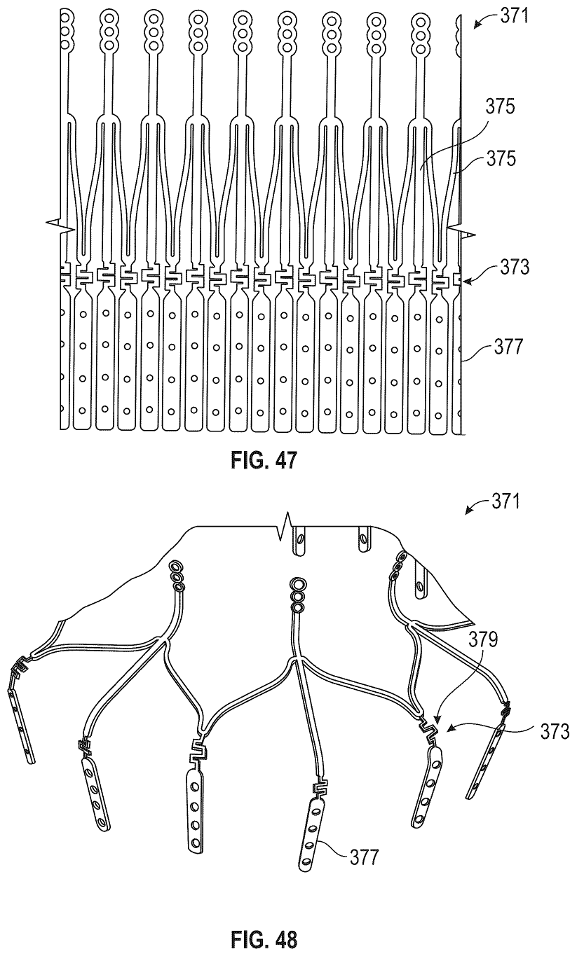

FIG. 47 illustrates a plan view of a frame 371 that may be utilized in examples herein. The frame 371 may comprise a proximal frame or a distal frame as utilized in examples herein. The frame 371 may include a flexible member 373 that may be utilized to allow the frame 371 to deflect. The flexible member 373, for example, may comprise an undulation or “rachis” feature in the frame 371 that may enhance the flexibility of the frame at the position of the flexible member 373. The flexible member 373 may couple to struts 375 of the frame 371 that may support the flexible member 373 and place the flexible member 373 at a desired position.

The flexible member 373 may couple to arms 377 that may comprise straightened plates that may support a skirt or other sealing body.

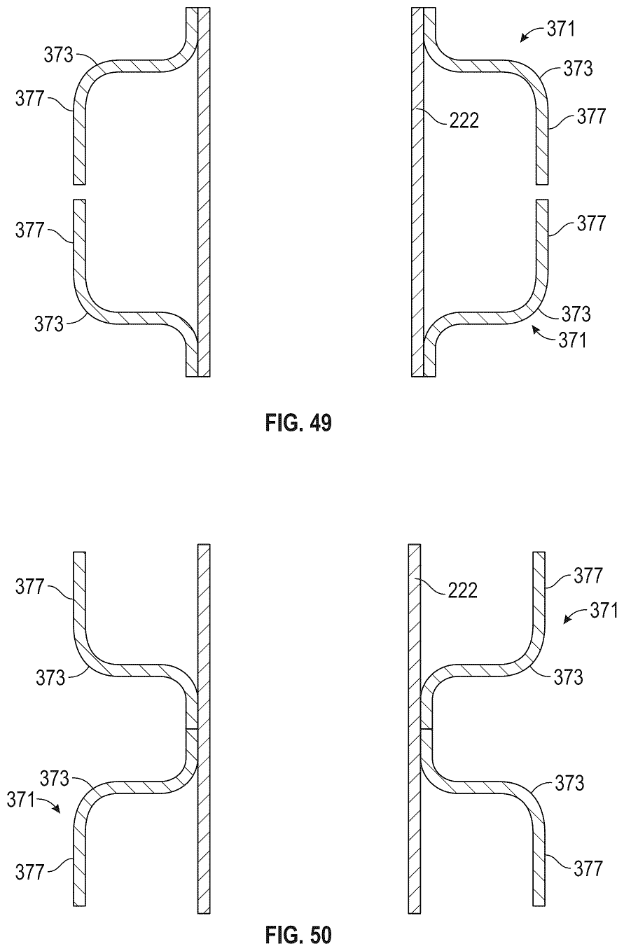

FIG. 48 illustrates a perspective view of the frame 371 formed into a shape including a shoulder 379. The flexible member 373 may be positioned at the shoulder 379 of the frame 371. The arms 377 may extend distally from the flexible member 373. In an example in which the frame 371 is used as a distal frame, the arms 377 may extend proximally from the flexible member 373 (an inverted configuration than shown in FIG. 48). The frame 371 may be utilized in examples herein. For example, referring to FIG. 49, the frame 371 may be utilized in a configuration as shown in FIG. 34. Further, referring to FIG. 50, the frame 371 may be utilized in a configuration as shown in FIG. 46. The shoulders disclosed herein may include the flexible member. One or more of a proximal frame or a distal frame may include a flexible member configured to allow a respective one of the proximal frame or the distal frame to deflect.

The flexible member 373 may be utilized to allow the frame 371 to conform to shapes of heart valve annuli that may be non-uniformly circular. For example, the frame 371 may be configured to conform to oval or “D” shaped annular shapes, among other forms of non-uniformly circular annuli. The flexible member 373 may enhance the compliance of the frame 371 to improve sealing with the heart valve annulus.

The examples of FIGS. 34-50 may include frames configured to self-expand to deploy to a deployment site such as a heart valve annulus. In examples, however, an inflatable body, such as a balloon, may be utilized to expand at least a portion of a prosthetic valve. For example, referring to the examples of FIGS. 34-50, the outer anchoring body may comprise a self-expanding frame. The inner body or inner frame may comprise a body configured to be expanded via an expansion mechanism or mechanical actuation. Using this arrangement, a two stage or multi-stage deployment accordingly may be advantageously used. More specifically, the outer anchoring body may be allowed to self-expand and an inflatable balloon may then be utilized to expand the inner frame. The outer anchoring body may be allowed to self-expand by retracting an outer sheath or by removing any other type of constraining device, such as, for example, a suture or ring.

In a sequence as shown in FIGS. 37-39, for example, the proximal and distal outer anchoring frames may be sequentially released and allowed to self-expand. The inner frame may then be expanded using an inflatable body, such as a balloon, which may be mounted on a distal end portion of the delivery catheter. Such multi-stage deployment may improve the control over the release of the implant and may provide other benefits including an enhanced strength of expansion due to the force provided by the inflatable body. In one advantage, the initial expansion of the outer frame portion (or portions) allows for partial anchoring of the prosthetic valve before the inner frame is expanded. This can significantly enhance the user's ability to precisely position the prosthetic valve before full deployment. This hybrid combination also provides the anchoring advantages of a self-expanding outer frame with the reliability advantages of a more rigid balloon-expandable inner frame. A more rigid inner frame maintains a circular profile and provides a stronger and more predictable structure for the tissue (e.g., pericardial tissue) valve.

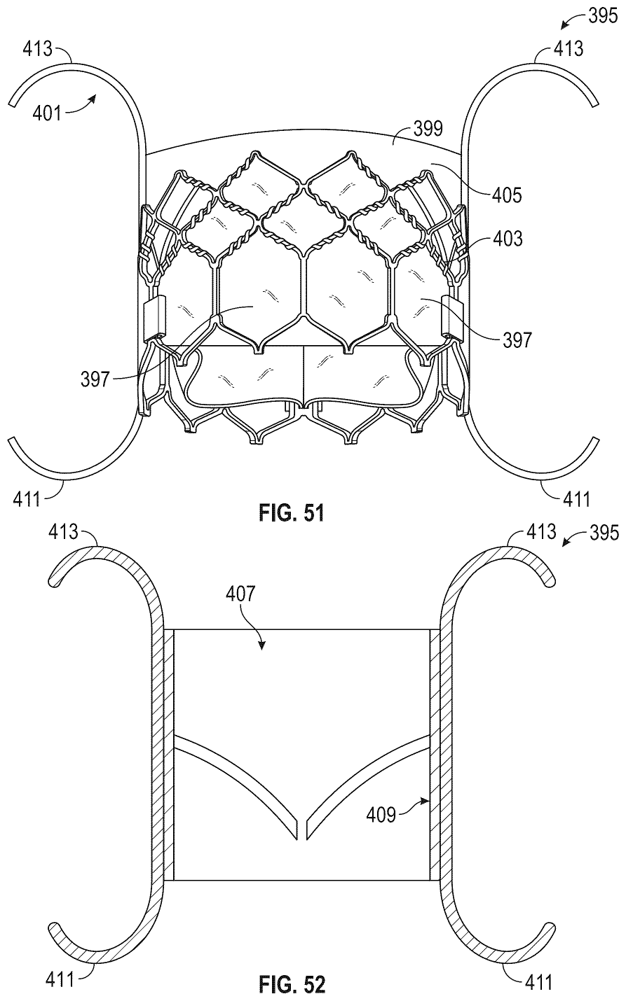

FIG. 51 further illustrates an example including a prosthetic valve 395 having a plurality of prosthetic valve leaflets 397 and an inner valve body 399 supporting the plurality of prosthetic valve leaflets 397 and configured to be expanded with an inflatable body, such as a balloon. The prosthetic valve 395 may include a self-expanding outer valve body 401 that may be configured to extend radially outward from the inner valve body 399.

The inner valve body 399 may include a frame 403 and in examples may include a sealing body 405 such as a skirt that may be utilized to seal fluid flow around a flow channel 407 (marked in FIG. 52) that the inner valve body 399 may surround. The prosthetic valve leaflets 397 may be positioned within the flow channel 407. The flow channel 407 may be configured to receive the inflatable body. The frame 403 may include a plurality of struts and may be configured to expand radially outward from an undeployed configuration to a deployed configuration.

The frame 403 may be configured to expand such that a length of the frame 403 decreases as a diameter of the frame 403 increases upon the frame expanding radially outward with the inflatable body. An inflatable body, such as a balloon, may be positioned within the flow channel 407 and expanded to provide a radially outward force against an interior surface 409 of the frame 403 to expand the frame 403. The frame 403 may be configured to expand radially outward with the inflatable body.

The frame 403 may have a cylindrical shape and may provide a cylindrical shape for the flow channel 407. In examples, other shapes of frames 403 may be utilized as desired.

The frame 403 may comprise a relatively stiff body and may be less flexible than the self-expanding outer valve body 401 in examples. The frame 403 may be made of a metal material such as cobalt chromium, or other forms of metals, and may retain its shape upon being expanded by an inflatable body.

The self-expanding outer valve body 401 may have a variety of forms. As shown in FIGS. 51-54, the self-expanding outer valve body 401 may comprise one or more anchors for anchoring to the heart valve, which may comprise at least one distal or ventricular anchor 411 or may comprise at least one proximal or atrial anchor 413 or may comprise a combination of distal anchors 411 and proximal anchors 413. The proximal anchors 413 may be positioned on an inflow side of a heart valve. FIG. 51, for example, illustrates a combination of distal anchors 411 and proximal anchors 413. Each of the anchors 411, 413 may extend radially outward from the inner valve body 399. The distal anchors 411 may be spaced circumferentially from each other and the proximal anchors 413 may be spaced circumferentially from each other. The distal anchors 411, in examples, may be configured to hook over a distal tip of a native heart valve leaflet as desired.

The self-expanding outer valve body 401 may be configured to self-expand upon being released by a retaining body. The retaining body may comprise a capsule of a delivery system or may have another form such as sutures or another body. In examples, the retaining body may be configured to release upon a force being applied by the inflatable body. For example, the inflatable body may apply a force radially outward to the inner valve body 399 that may break or otherwise release the retaining body and may allow the outer valve body 401 to self-expand.

In examples, the self-expanding outer valve body 401 may be more compliant than the inner valve body 399. The self-expanding outer valve body 401 may be more flexible and as such able to have greater conformity to an irregular or otherwise non-uniformly circular shape of a native valve annulus. The self-expanding outer valve body 401 may be able to fill and conform to a shape of a native valve annulus having an oval shape, or a “D” shape, or another non-uniformly circular shape of a native valve annulus.

Further, the self-expanding outer valve body 401 may be able to conform to and seal an annulus affected by annular calcification. Annular calcification (or “MAC” when present with the mitral valve) may produce an irregular shape of a heart valve annulus that may render it difficult for a cylindrical inner valve body 399 to anchor to and scal with. As such, the self-expanding outer valve body 401 may more easily anchor to a heart valve affected with calcification or otherwise having an irregular shape.

The inner valve body 399 may be expandable via an inflatable body to provide a radially outward force that may press against calcification, to improve anchoring to the annulus affected by the calcification. The expansion of the inner valve body 399 may provide a force of expansion and the compliance of the self-expanding outer valve body 401 may provide improved anchoring and sealing with an annulus affected with calcification or otherwise having an irregular shape.

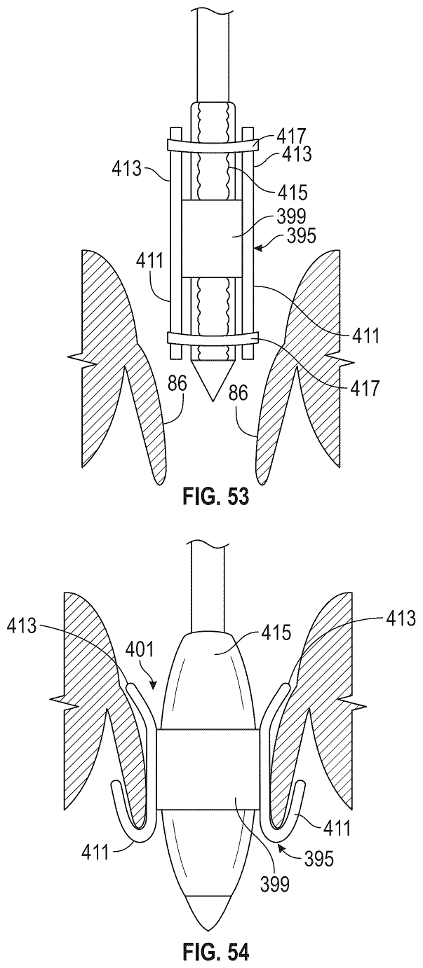

FIGS. 53 and 54, for example, illustrate an exemplary deployment of the prosthetic valve 395. The prosthetic valve 395 in FIG. 53 may be positioned upon an inflatable body 415 that may be part of a delivery apparatus. The inflatable body 415, for example, may pass through the flow channel 407 shown in FIG. 52. The inflatable body 415 may be in a deflated state.

The prosthetic valve 395 may be in an undeployed configuration and the anchors 411, 413 may be straightened and may extend axially with the prosthetic valve 395. Retaining bodies 417 such as bands may extend over the anchors 411, 413 to prevent the self-expansion of the anchors 411, 413 to the deployed configuration shown in FIGS. 51 and 52 for example. The retaining bodies 417 may couple to tethers of the delivery apparatus that prevent the retaining bodies 417 from becoming free within the patient's body. Other forms of retaining bodies such as sutures, or a capsule of the delivery apparatus may retain the anchors 411, 413 in examples.

With the prosthetic valve 395 in the desired position, the inflatable body 415 may be inflated to expand the inner valve body 399. FIG. 54, for example, illustrates the inflatable body 415 expanded via liquid or another substance to increase the diameter of the inflatable body 415. The force of the inflatable body 415 may break the retaining bodies 417, which may tether to a portion of the delivery apparatus for retrieval. The release of the retaining bodies 417 may allow the self-expanding outer valve body 401 to expand and may conform to the shape of the heart valve annulus. The self-expanding outer valve body 401 may be allowed to deflect to conform to a shape of an annulus of a heart valve, which may include any calcification of the heart valve. The distal anchors 411 may anchor in position, and the proximal anchors 413 may anchor in position. The inflatable body 415 may apply a force radially outward to press against the heart valve and press against any calcification that may be present. The inflatable body 415 and delivery system may be removed to leave the prosthetic valve 395 implanted.

The resulting implanted valve may have a uniformly circular flow channel 407, which may be desirable to enhance fluid flow through the valve, yet may have a non-uniformly circular shape of the self-expanding outer valve body 401.

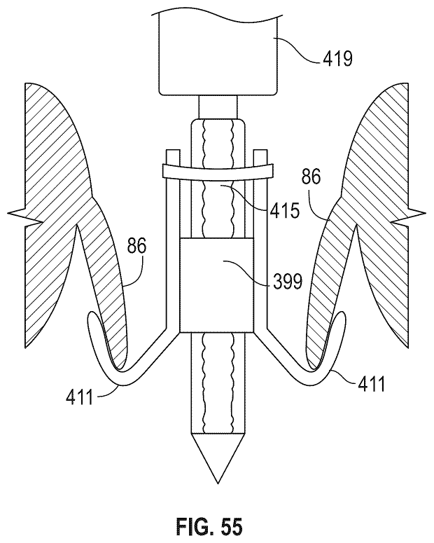

FIG. 55 illustrates a form of deployment in which all or a portion of the self-expanding outer valve body 401 may be partially or fully deployed prior to expansion of the inflatable body 415. As shown in FIG. 55, the distal anchors 411, or another portion of the self-expanding outer valve body 401 may be released. Distal anchors 411, for example, may anchor to heart valve leaflets 86 prior to expansion of the inflatable body 415. A capsule 419, for example, may be retracted to allow the distal anchors 411 to deploy and another form of retaining body may hold the proximal anchors until the inflatable body is expanded. A clinician accordingly may determine if anchoring or sealing has occurred prior to expansion of the inflatable body 415. The clinician may adjust the position of the outer valve body 401 if desired, prior to expansion of the inner valve body 399. With the self-expanding outer valve body 401 in a desired position, the inflatable body 415 and the inner valve body 399 may be expanded with a radially outward force.

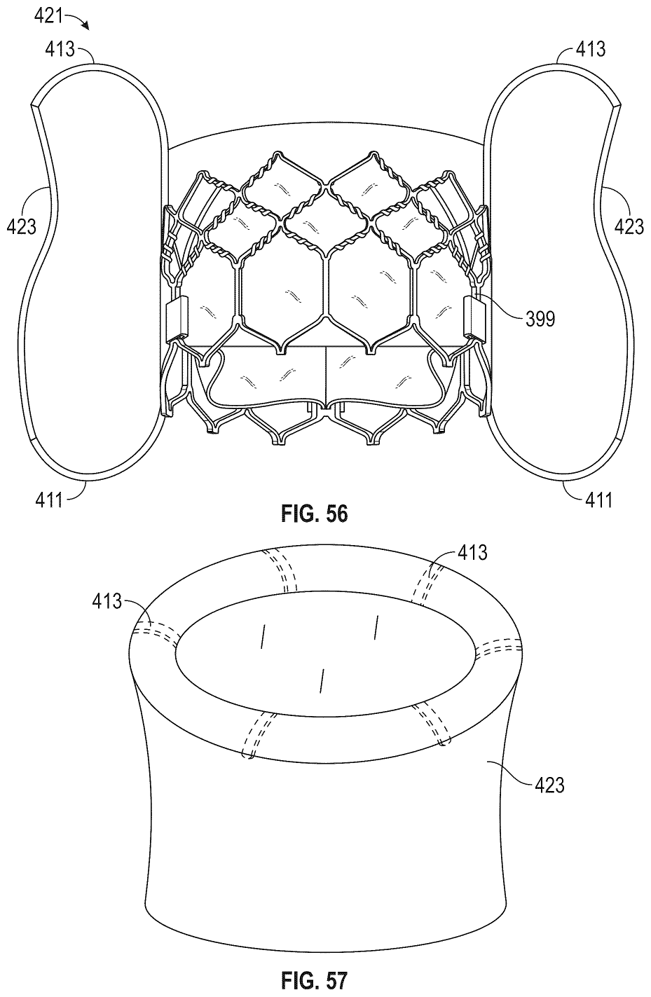

In examples, other configurations of self-expanding outer valve bodies 401 may be utilized. FIGS. 56 and 57, for example, illustrate an example of a prosthetic valve 421 in which a scaling body, such as a sealing skirt 423, may extend from the proximal anchors 413 to the distal anchors 411. The sealing body may be for sealing with the heart valve. The sealing skirt 423 may be flexible and configured to deflect to fit within the valve annulus. The sealing skirt 423 may form a sheath extending circumferentially about the inner valve body 399 and may extend over the upper end of the proximal anchors 413 as shown in FIG. 57, and over the lower end of the distal anchors 411. The sealing skirt 423 may be stretchable to conform to a shape of an annulus.

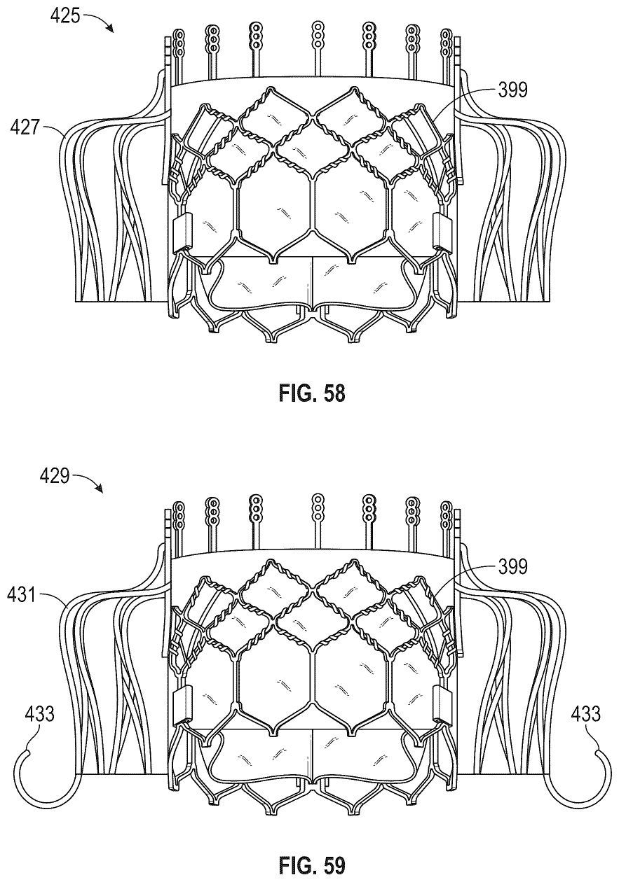

FIG. 58 illustrates an example of a prosthetic valve 425 in which the self-expanding outer valve body 427 comprises an outer frame. The outer frame may be a self-expanding frame configured to expand radially outward. The outer frame may be configured to be compliant to deflect to conform to a shape of an annulus of a heart valve. An inner frame may be less flexible than the self-expanding outer frame.

FIG. 59 illustrates an example of a prosthetic valve 429 in which the self-expanding outer valve body 431 comprises an outer frame including distal anchors 433. The outer frame may be configured to be compliant to conform to a shape of an annulus. The distal anchors 433 may be configured to hook around distal tips of leaflets of a heart valve or may engage calcification of a heart valve annulus.

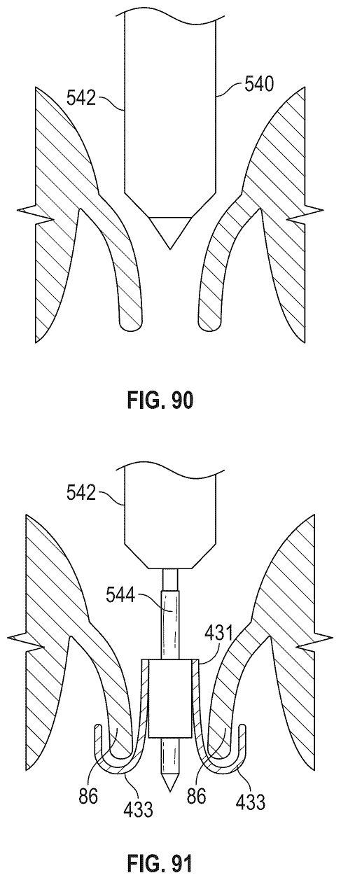

FIGS. 90-94 illustrate an exemplary deployment of the prosthetic valve 429. Referring to FIG. 90, the prosthetic valve 429 may be coupled to a delivery apparatus 540 that may approach an implantation site. The prosthetic valve 429 may be positioned within the delivery apparatus 540 (e.g., within a capsule of the delivery apparatus 540) or may otherwise be coupled to the delivery apparatus 540. The delivery apparatus 540 may include a retaining body in the form of a capsule 542, or another form of retaining body may be utilized. A capsule may extend over any example of prosthetic valve disclosed herein. The capsule may extend over an inflatable body in examples.

Referring to FIG. 91, the capsule 542 may be retracted to allow the self-expanding outer valve body 431 to expand. The distal anchors 433 may hook around the distal tips of leaflets 86 or may otherwise be positioned. A clinician may determine if a desired position of the self-expanding outer valve body 431 and distal anchors 433 has occurred. The position of the prosthetic valve 429 may be varied as desired.

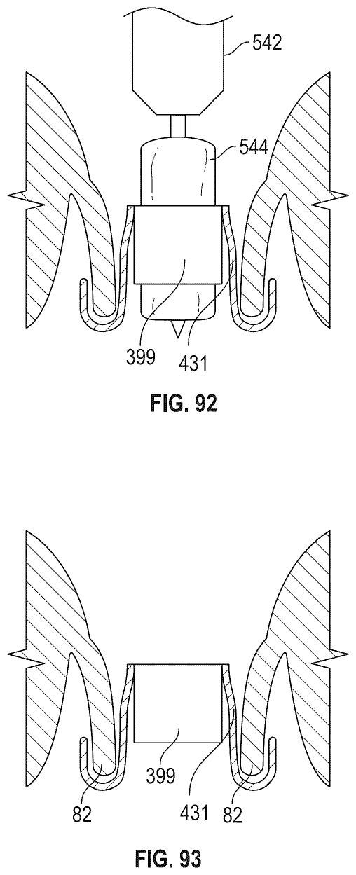

Referring to FIG. 92, at a desired time the inflatable body 544 may be inflated to expand the inner valve body 399. The force of the inflatable body 544 against the interior surface of the inner valve body 399 may expand the inner valve body 399. Further expansion of the self-expanding outer valve body 431 may also occur. The inflatable body 544 and delivery apparatus 540 may be withdrawn to leave the prosthetic valve 429 implanted as shown in FIG. 93.

Other forms of prosthetic valves disclosed herein may comprise valves that include self-expanding features and expansion via actuation such as an inflatable body (e.g., a balloon). For example, any configuration of prosthetic valve shown in FIGS. 34-50 or otherwise disclosed herein may have such a configuration.

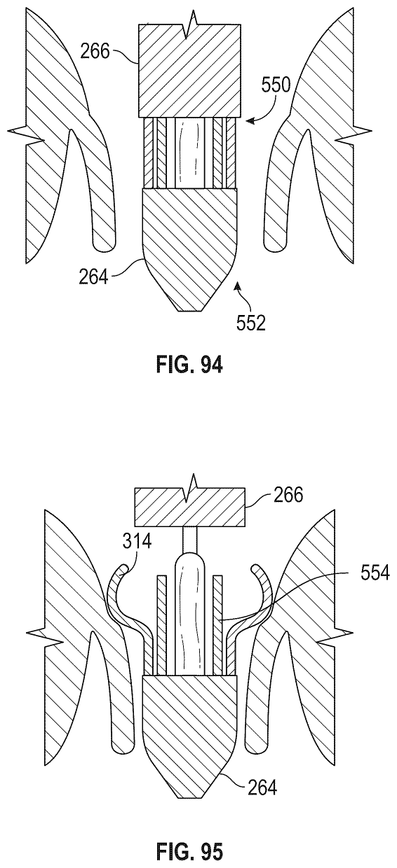

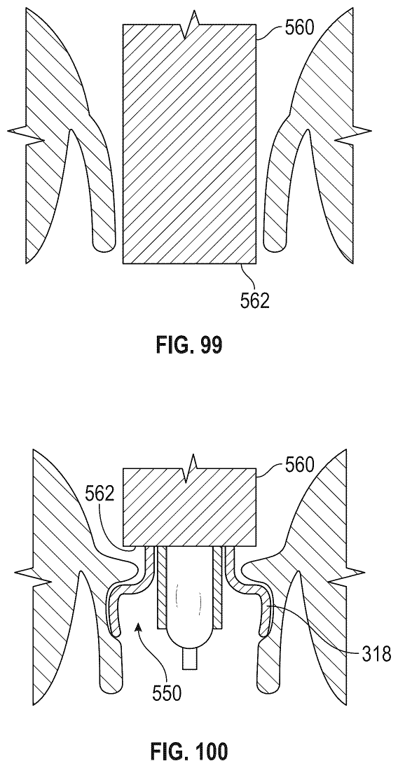

FIGS. 94-98, for example, illustrate an exemplary deployment of a configuration of prosthetic valve shown in FIG. 46, in which the inner valve body or inner frame 224 is expandable via actuation such as an inflatable body (e.g., a balloon). For example, referring to FIG. 94, such a prosthetic valve 550 may be coupled to a delivery apparatus 552 that may approach an implantation site. The prosthetic valve 550 may be positioned within the delivery apparatus 552 (e.g., within a capsule of the delivery apparatus 552) or may otherwise be coupled to the delivery apparatus 552. The delivery apparatus 552 may include a retaining body in the form of a capsule, or another form of retaining body may be utilized. A split capsule design (for example, as shown in FIGS. 36-39) may be utilized or a unitary capsule may be utilized (as represented in FIGS. 99-102 for example).

The delivery apparatus 552 may be placed in a desired position and a portion of the prosthetic valve 550 may be allowed to expand. For example, as shown in FIG. 95, a proximal portion 266 of the capsule may be retracted to allow the proximal frame 314 of the anchoring body to expand. The proximal frame 314 may be self-expanding. The proximal frame 314 may be placed in a desired position relative to the heart valve and particularly relative to the annulus of the heart valve.

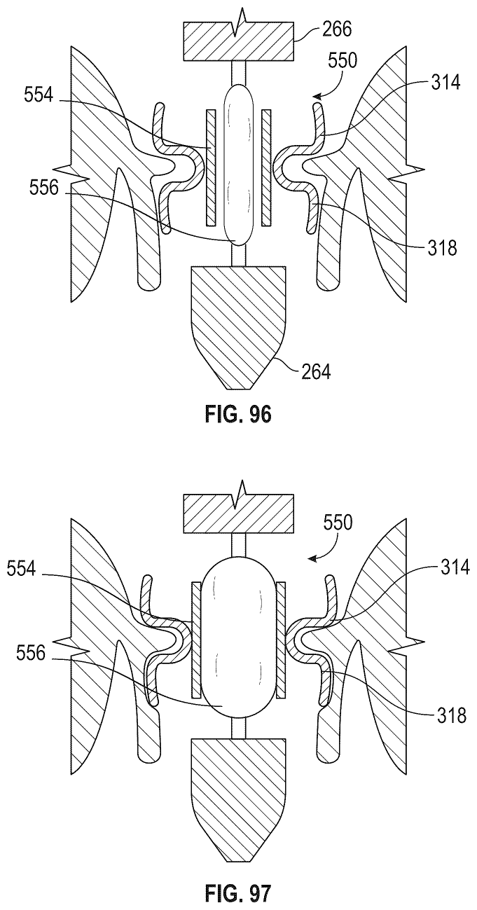

Upon a determination that the proximal frame 314 is in a desired position, the distal frame 318 may be allowed to expand. For example, as shown in FIG. 96, the distal portion 264 of the capsule may be advanced distally to allow the distal frame 318 to expand. The prosthetic valve 550 may not yet be fully expanded because the inner valve body or inner frame 554 (corresponding to the inner valve body or inner frame 224 shown in FIG. 46) has not yet been expanded. The inner frame 554 may be positioned upon the inflatable body 556 in such a configuration. As such, additional movements of the prosthetic valve 550 may be made to place the prosthetic valve 550 in the desired position (e.g., axial movements or lateral movements as desired). The proximal frame 314 and distal frame 318 may capture a portion of the heart valve (e.g., a portion of the annulus) between the frames 314, 318 at this configuration.

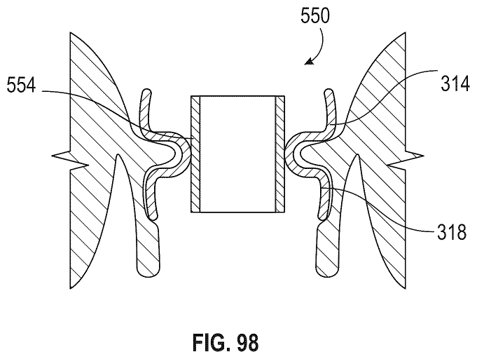

Referring to FIG. 97, at a desired time the inflatable body 556 may be inflated to expand the inner frame 554. The force of the inflatable body 556 against the interior surface of the inner frame 554 may expand the inner frame 554. Further expansion of the self-expanding outer valve body (including the proximal frame 314 and distal frame 318) may also occur. The inflatable body 556 and delivery apparatus 552 may be withdrawn to leave the prosthetic valve 550 implanted as shown in FIG. 98.

The deployment sequence may utilize a single capsule or unitary capsule in examples. For example, referring to FIG. 99, a delivery apparatus may utilize a capsule 560 that may be configured to deploy the prosthetic valve 550 from a distal end portion 562 of the capsule 560. The capsule 560 may be retracted proximally to release the prosthetic valve 550 from the capsule 560.

For example, referring to FIG. 100, the capsule 560 has retracted to partially deploy the distal frame 318 of the prosthetic valve 550. The distal frame 318 may be placed in a desired position relative to the heart valve and particularly relative to the annulus of the heart valve.

Upon a determination that the distal frame 318 is in a desired position, the proximal frame 314 may be allowed to expand. For example, as shown in FIG. 101, the distal end portion 562 of the capsule 560 may be retracted proximally to allow the proximal frame 314 to expand. The prosthetic valve 550 may not yet be fully expanded because the inner valve body or inner frame 554 (corresponding to the inner valve body or inner frame 224 show in FIG. 46) has not yet been expanded. The inner frame 554 may be positioned upon the inflatable body 556 in such a configuration. As such, additional movements of the prosthetic valve 550 may be made to place the prosthetic valve 550 in the desired position (e.g., axial movements or lateral movements as desired). The proximal frame 314 and distal frame 318 may capture a portion of the heart valve (e.g., a portion of the annulus) between the frames 314, 318 at this configuration.

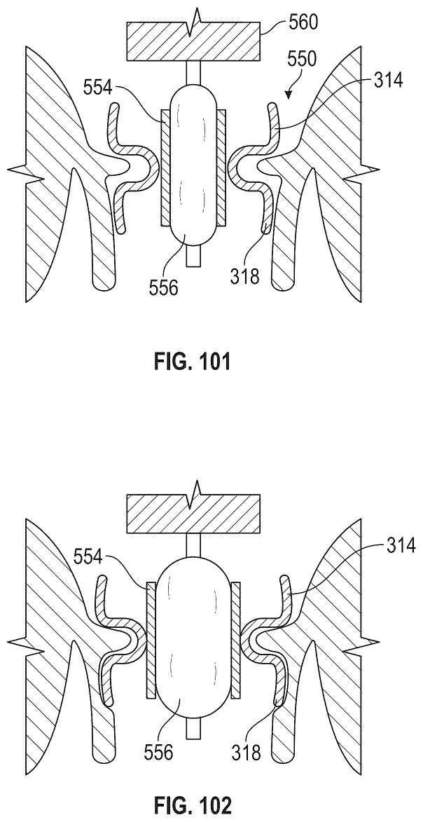

Referring to FIG. 102, at a desired time the inflatable body 556 may be inflated to expand the inner valve body 554. The force of the inflatable body 556 against the interior surface of the inner valve body 554 may expand the inner valve body 554. Further expansion of the self-expanding outer valve body (including the proximal frame 314 and distal frame 318) may also occur. The inflatable body 556 and delivery apparatus may be withdrawn to leave the prosthetic valve 550 implanted in a similar configuration as shown in FIG. 98.

Other forms of prosthetic valves shown in FIGS. 34-50 or otherwise disclosed herein may be deployed in a similar manner.

The self-expanding bodies and frames may be made of a shape memory material in examples, such as nitinol. Other shape memory materials may be utilized as desired. Valve frames and elongate anchor arms may be made from a shape memory material, for example. Other components may be made from a shape memory material in examples.

Various other modification of the examples herein may be provided. A combination of an inner valve body that is expandable with an inflatable body, and a self-expanding outer valve body may be utilized solely or in combination with any example disclosed herein.

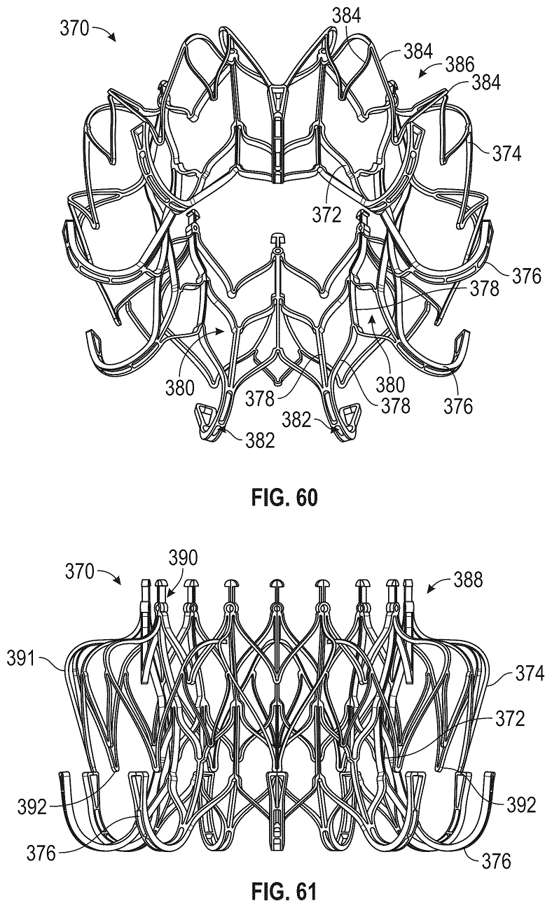

FIGS. 60-61 illustrates a frame of a prosthetic valve 370 including an inner frame 372 and an outer frame 374 positioned radially outward of the inner frame 372. The inner frame 372 may support a plurality of prosthetic valve leaflets and may include a plurality of anchors 376 for anchoring the prosthetic valve 370 to a heart valve.

The inner frame 372 may include a plurality of struts 378 separated by a plurality of openings 380. The inner frame 372 may be configured to move from an uncompressed configuration to a compressed or crimped configuration upon a radially inward force being applied to the inner frame 372. Upon such compression, a diameter of the inner frame 372 may decrease and a length of the inner frame 372 may increase. The spacing between the struts 378 formed by the plurality of openings 380 may allow such compression of the inner frame 372.

In examples, the anchors 376 may extend distally from the inner frame 372 and may be configured to hook around leaflets of a heart valve to anchor to the heart valve. The anchors 376 may be configured to be positioned radially outward of heart valve leaflets to anchor the prosthetic valve 370 to the heart valve.

The anchors 376, in examples, may include cut outs 382 that may be utilized to increase flexibility of the anchors 376. The cut outs 382 may extend along a length of the anchors 376.

The outer frame 374 may include a plurality of struts 384 separated by a plurality of openings 386. Referring to FIG. 61, the outer frame 374 may include a proximal portion 388 that is coupled to a proximal portion 390 of the inner frame 372. The outer frame 374 may extend radially outward to form a shoulder 391 and may extend to distal apices 392 of the outer frame 374. The outer frame 374 in examples may be covered with a sealing skirt and may be utilized to seal with a heart valve.

In examples, the prosthetic valve 370 may be configured to be compressed radially inward to crimp the prosthetic valve 370. The radially inward compression may reduce the diameter of the outer frame 374 and the inner frame 372, and may result in an elongation of the outer frame 374 and inner frame 372.

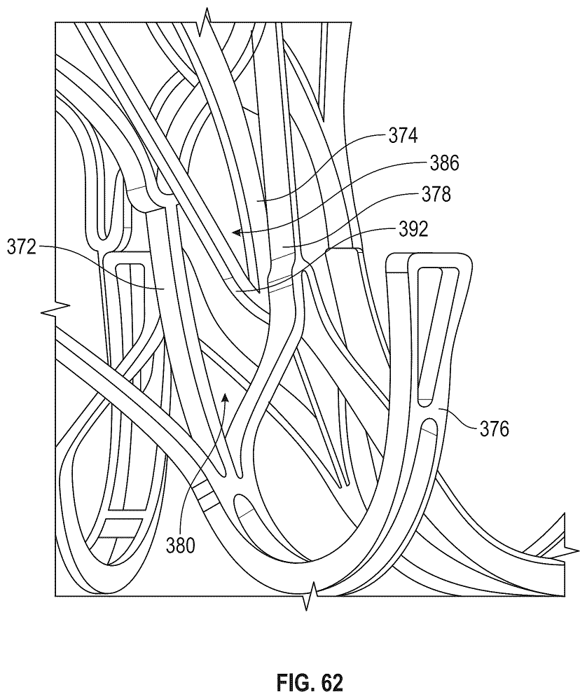

An issue may arise with a radial compression of the outer frame 374. Referring to FIG. 62, a portion of the outer frame 374 may interlace with the inner frame 372 upon radial compression of the outer frame 374. A portion of the outer frame 374 may pass through an opening 380 of the inner frame 372 and may be held by the inner frame 372. For example, as shown in FIG. 62, one or more of the apices 392 of the outer frame 374 may pass into an opening 380 of the inner frame 372 and may be held behind a strut 378 of the inner frame 372. Other interlacing between the outer frame 374 and the inner frame 372 may occur, for example, with the anchors 376 of the inner frame 372 or other portions of the inner frame 372. Interlacing of the outer frame 374 with the inner frame 372 may be undesirable because upon deployment, the outer frame 374 may not fully separate from the inner frame 372 and may not deploy properly. For example, in an example in which an outer frame 374 is utilized for sealing, an improper seal may be provided with the heart valve due to the interlacing of the outer frame 374 with the inner frame 372.

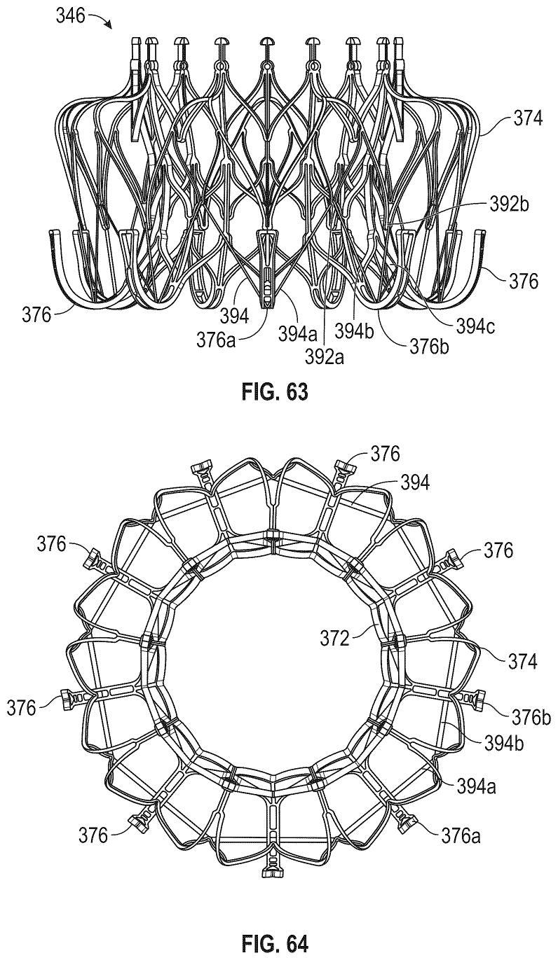

FIG. 63 illustrates an example of the present disclosure including one or more tethers 394 coupling the outer frame 374 to the inner frame 372 and configured to impede movement of the outer frame 374 radially inward to reduce interlacing of the outer frame 374 with the inner frame 372.

Each of the one or more tethers 394, for example, may include a first end portion coupled to the outer frame 374 and a second end portion coupled to the inner frame 372. In examples, as shown in FIG. 63, the second end portion of the one or more tethers 394 may be coupled to one or more of the plurality of anchors 376. In examples, the first end portion of the one or more tethers 394 may be coupled to one or more of the apices 392 of the outer frame 374. The one or more tethers 394 in examples may be held taut between the outer frame 374 and the inner frame 372 in a configuration shown in FIG. 63 for example.

The one or more tethers 394 may extend circumferentially about the inner frame 372 in examples, and may alternate between a coupling to the inner frame 372 and a coupling to the outer frame 374. For example, as shown in FIG. 63, a first tether 394a may extend circumferentially and proximally from an anchor 376a to an apex 392a of the outer frame 374. The first tether 394a may extend diagonally. A second tether 394b may extend circumferentially and distally from the apex 392a to the anchor 376b. The second tether 394b may extend diagonally. A third tether 394c may extend proximally from the anchor 376b to the apex 392b of the outer frame 374. In examples, a fourth tether may be provided that may extend distally from the apex 392b to the anchor 376b. The pattern of tethers may repeat circumferentially about the inner frame 372, as shown in FIGS. 63 and 64. In examples, a single tether may be routed between the inner frame 372 and the outer frame 374, or in examples multiple tethers may be utilized. The routing of the one or more tethers 394 may be varied in examples.

The one or more tethers 394 may be configured to impede movement of the outer frame 374 radially inward to reduce interlacing of the outer frame 374 with the inner frame 372 during a crimping operation applied to the prosthetic valve 396. As the outer frame 374 is compressed radially inward, the one or more tethers 394 may reduce the radially inward movement of the outer frame 374 by applying a tension against the outer frame 374. As such, the outer frame 374 may have a reduced possibility of interlacing with the inner frame 372.

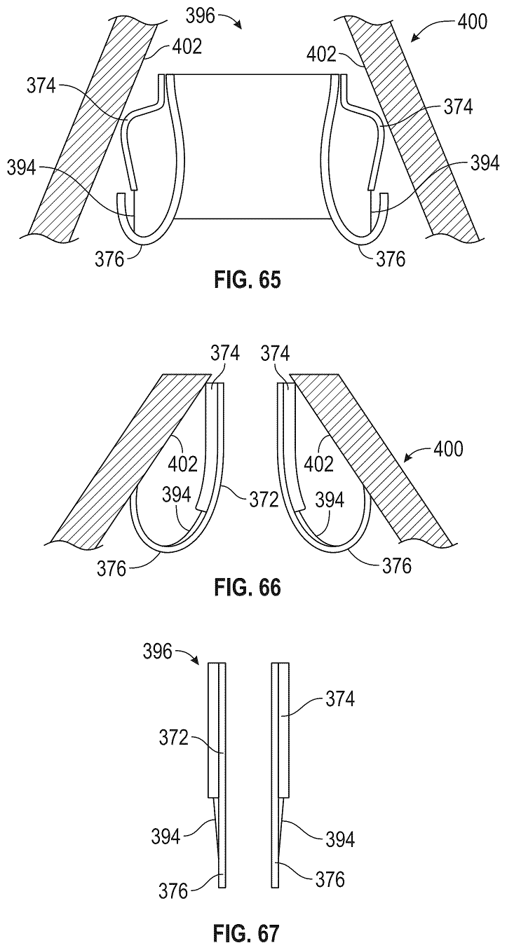

FIGS. 65 and 66, for example, illustrate a crimping operation that may be applied to the prosthetic valve 396. A crimping operation may include compressing the prosthetic valve 396 radially inward. In examples, one or more surfaces may be utilized to compress the prosthetic valve 396 radially inward. The one or more surfaces may be part of a crimping device 400 that may be utilized to crimp the prosthetic valve 396. The one or more surfaces in examples may be movable surfaces that apply a compressive force to the prosthetic valve 396 by moving radially inward relative to each other. In examples, the one or more surfaces may comprise tapered surfaces 402 that may be angled to cause the prosthetic valve 396 to be compressed as the prosthetic valve 396 is slid relative to the tapered surfaces 402. For example, as shown in FIG. 65, the prosthetic valve 396 may be slid in the direction towards the narrowing of the tapered surfaces 402. The tapered surfaces 402 may press radially inward against the prosthetic valve 396 to crimp the prosthetic valve 396.

As the outer frame 374 is compressed radially inward, the tethers 394 may impede the radially inward movement of the outer frame 374 to reduce interlacing. As shown in FIG. 66, for example, as the outer frame 374 moves radially inward towards the inner frame 372, the tethers 394 may apply a tension to reduce the possibility of the outer frame 374 interlacing with the inner frame 372. The tethers 394 in examples may reduce a twisting of the outer frame 374 that may result in interlacing of the outer frame 374 with the inner frame 372.

A crimping operation may proceed with the prosthetic valve 396 continuing to be compressed by the tapered surfaces 402. The anchors 376 may be moved to a longitudinal configuration due to the compression of the prosthetic valve 396. FIG. 67, for example, illustrates the prosthetic valve 396 having been compressed in the crimping operation with the diameter of the prosthetic valve 396 having been reduced and outer frame 374 and inner frame 372 both elongating. The tethers 394 may continue to apply a tension that reduces interlacing of the outer frame 374 with the inner frame 372.

Upon deployment, the outer frame 374 and inner frame 372 may both expand radially outward to the configuration shown in FIG. 41 for example.

The features of the prosthetic valve of FIGS. 60-67 may be utilized with any example of prosthetic valve disclosed herein. For example, the features of the prosthetic valve of FIGS. 60-67 may be utilized with any of the prosthetic valves shown in FIGS. 34-50, among other valves disclosed herein. The features of the prosthetic valve of FIGS. 60-67 may be utilized, for example, to impede movement of an anchoring body of any of FIGS. 60-67 radially inward to reduce interlacing of the anchoring body with an inner frame. Other uses of the features of the prosthetic valve of FIGS. 60-67 may be provided.

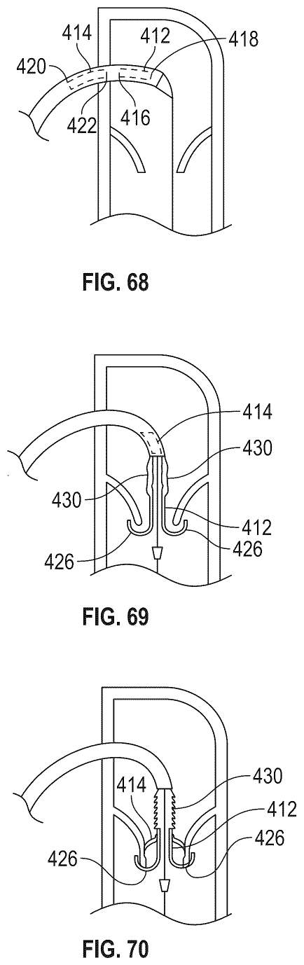

FIGS. 68-73 illustrate an example of a prosthetic valve 410 including an inner frame 412 that supports a plurality of prosthetic valve leaflets and an outer frame 414 that is configured to be positioned radially outward of the inner frame 412 and configured to be assembled to the inner frame 412 within a patient's body.

The inner frame 412 and outer frame 414, for example, may be unassembled with respect to each other and held in an undeployed configuration upon delivery towards an implantation site. FIG. 68 illustrates such a configuration, with the inner frame 412 and outer frame 414 both positioned in an undeployed configuration within a delivery apparatus. The delivery apparatus may be configured similarly as the delivery apparatus 62 shown in FIG. 5 for example.

The inner frame 412 may be held in a compressed configuration, with the outer frame 414 being longitudinally adjacent to the inner frame 412 while held within the delivery apparatus. The inner frame 412 for example, may include a proximal end 416 and a distal end 418. The outer frame 414 may include a proximal end 420 and a distal end 422. The distal end 422 of the outer frame 414 may be positioned adjacent to the proximal end 416 of the inner frame 412 when both the outer frame 414 and the inner frame 412 are both in the undeployed configuration.

Upon deployment, the inner frame 412 may be released from the delivery apparatus. FIG. 69, for example, illustrates the inner frame 412 having been released. The inner frame 412 may expand radially outward in examples, or may remain in a compressed configuration for expansion via another mechanism such as a balloon expandable frame or a mechanically expandable frame. In examples, the inner frame 412 may be configured similarly as examples of inner frames disclosed herein, which may include an inner frame 372 shown in FIGS. 60 and 61 for example, among others.

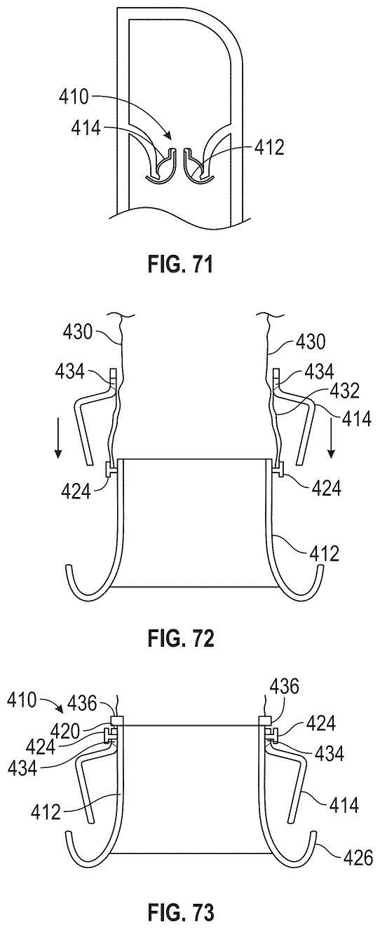

In examples, the inner frame 412 may include engagement bodies 424 (marked in FIG. 72) that may be utilized to secure the inner frame 412 to the outer frame 414. The engagement bodies 424 may comprise couplers or other bodies for engagement between the inner frame 412 and outer frame 414. The engagement bodies 424 may be configured to secure the inner frame 412 to the outer frame 414 upon contact between the inner frame 412 and outer frame 414. In examples, the engagement bodies 424 may comprise pins, apertures, snaps, magnetic couplers, clips, or other forms of bodies for engagement between the inner frame 412 and outer frame 414. The engagement bodies 424 may have a shape that mates with a shape of a corresponding engagement body on the outer frame 414, and in examples, may be configured to lock upon contact to reduce the possibility of the inner frame 412 releasing from the outer frame 414 undesirably.

In examples, the inner frame 412 may include anchors 426 that may be utilized to anchor the inner frame 412 and the prosthetic valve 410 to a desired implantation site. The anchors 426 in examples may be configured to hook around leaflets of a heart valve, similar to the anchors 376 shown in FIGS. 60 and 61, or may have another configuration as desired.

In examples, one or more tethers 430 may be utilized that may extend between the inner frame 412 and the outer frame 414. The tethers 430 may be utilized to guide the inner frame 412 relative to the outer frame 414 and may draw the outer frame 414 to the inner frame 412 within the patient's body. In examples, other forms of guide mechanisms may be utilized such as rails, sheaths, magnets, or other mechanism, to guide the inner frame 412 relative to the outer frame 414. In FIGS. 69 and 72, the one or more tethers 430 may comprise a cord looped with one end 432 of the cord coupled to the outer frame 414 and another end of the cord accessible by a user. The user may access the cord outside of the patient's body if desired. Referring to FIG. 72, the cord may be looped around the inner frame 412 such that pulling an end of the cord may draw the outer frame 414 towards the inner frame 412.

The outer frame 414 may be configured similarly as the outer frame 374 shown in FIGS. 60 and 61, or may have another configuration as desired. Similar to the inner frame 412, the outer frame 414 may include engagement bodies 434 (marked in FIG. 72) that may be utilized to secure the inner frame 412 to the outer frame 414. The engagement bodies 434 may be configured in a manner disclosed with respect to the engagement bodies 424. In examples, the engagement bodies 434 may have a shape that mates with a shape of a corresponding engagement body 424 on the inner frame 412, and in examples, may be configured to lock upon contact to reduce the possibility of the inner frame 412 releasing from the outer frame 414 undesirably.

Referring to FIG. 69, the outer frame 414 may be deployed from the delivery apparatus and the tethers 430 may be utilized to draw the outer frame 414 to the inner frame 412 within the patient's body. FIG. 72, for example, illustrates the outer frame 414 being drawn to the inner frame 412.

Referring to FIG. 70, the outer frame 414 may continue to be drawn to the inner frame 412 until the outer frame 414 is assembled to the inner frame 412. The outer frame 414 may be assembled to the inner frame 412, with the outer frame 414 being positioned radially outward of the inner frame 412. The proximal end of the inner frame 412 may be positioned adjacent to the proximal end of the outer frame 414, in a configuration shown in FIG. 73 for example.

The outer frame 414 may be drawn to the inner frame 412 via tensioning of the one or more tethers 430. The tethers 430 may be tensioned to cause the engagement bodies 424, 434 to engage with each other and secure the inner frame 412 to the outer frame 414. FIGS. 72 and 73 illustrate the tethers 430 having been tensioned and the engagement bodies 424, 434 coupled to each other.

In examples, as shown in FIG. 70, a tether 430 may extend from the coupled outer frame 414 and inner frame 412. The tethers 430 may be cut with a cutting device to reduce the extent of the tether 430 extending from the prosthetic valve 410. Referring to FIG. 73, in examples, one or more locks 436 may be provided that may secure the inner frame to the outer frame within the patient's body. The locks 436 may couple to the tethers 430 in examples and may maintain a tension in the tether 430 that may secure the outer frame 414 to the inner frame 412. The locks 436 in examples may include a cutting device that may cut a portion of the tether extending from the prosthetic valve 410.

With the outer frame 414 assembled to the inner frame 412, the delivery apparatus may be withdrawn from the implantation site. The assembled prosthetic valve 410 may remain implanted within the patient's body as shown in FIG. 71 for example.

The prosthetic valve 410 may beneficially allow for a reduced diameter of the prosthetic valve 410 upon passage through the patient's body. The reduced diameter may reduce the diameter of the delivery apparatus utilized to deliver the prosthetic valve 410 to an implantation site and thus may reduce the size of the entry into a patient's body. The prosthetic valve 410 may be deployed in a multi-stage sequence as shown in FIGS. 68-71, with the frames being assembled within the patient's body.

In examples, the configuration of the frames may vary from the configuration shown in FIGS. 68-73. For example, the frames may be configured similarly as any other frame disclosed herein, among other forms of frames. Further, in examples, the apparatuses utilized for deployment may vary from the apparatuses shown in FIGS. 68-71. The methods utilized for deployment and a sequence of deployment may vary from the sequence shown in FIGS. 68-71. For example, an outer frame may be deployed prior to an inner frame in examples. Further, the position and configuration of the frames in the undeployed configuration and the deployed configuration may vary in examples.

The features of FIGS. 68-73 may be utilized with any example of prosthetic valve disclosed herein.

In examples, a prosthetic valve may be provided that may be configured to be deployed to a heart valve of a patient's heart. At least a portion of the prosthetic valve may have a dimension that has been reduced with electropolishing.

For example, referring to FIG. 74, a frame 440 may be provided for a prosthetic valve. The frame 440 is shown in FIG. 74 in a flattened configuration, yet may be provided in a cylindrical or other rounded configuration in examples. In examples, the frame 440 may be provided in a flattened configuration in the method disclosed herein. A frame that may be utilized may comprise any frame disclosed herein. Any frame of a prosthetic valve disclosed herein may have a dimension that has been reduced with electropolishing.

The frame 440 may comprise a plurality of struts 441a, b, c that may be joined at junctures. The struts 441a, b, c, may form a lattice structure for the frame 440 separated by a plurality of openings 442a, b, c. The struts 441a, b, c may be cut or laser cut from a body that may be unitary, such as a continuous cylinder 444 as shown in FIG. 79 for example. Such a cylinder 444 may be provided and then a cut pattern may be applied to form the configuration of the frame 440 shown in FIG. 74 for example, or any other configuration of frame desired.

The frame 440 may comprise a proximal portion or a valve frame 449 and may include a distal portion or one or more anchors 451 coupled to the valve frame 449. The one or more anchors may be for anchoring the prosthetic valve to a heart valve. The plurality of anchors 451 may be configured similarly to other examples of anchors disclosed herein, and may be configured to curve proximally to anchor to a native heart valve leaflet or other portion of a native heart valve. The anchors 451 may be configured to extend over a tip of a native heart valve leaflet for example.

The anchors 451 are shown in an elongated configuration in FIG. 74, yet may deflect proximally as shown in examples of anchors disclosed herein. The anchors 451 may be configured to extend in a proximal direction from a distal portion of the valve frame 449. The anchors 451 may be configured to be positioned radially outward of a native heart valve leaflet or may be positioned radially inward of a native heart valve leaflet for anchoring. Configurations of anchors for being positioned radially outward of a native heart valve leaflet or being positioned radially inward of a native heart valve leaflet are disclosed herein.

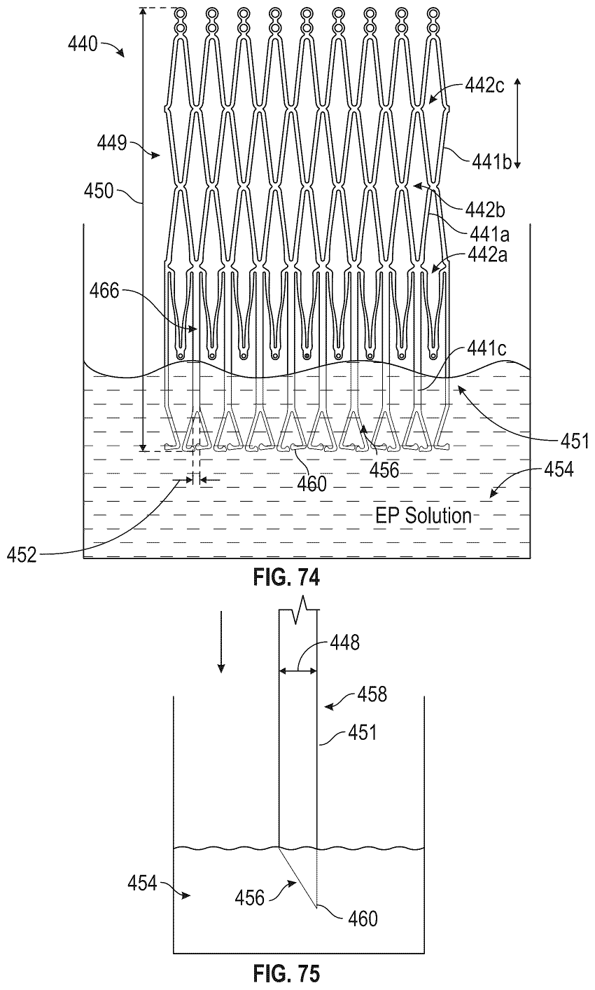

The body, such as the continuous cylinder 444, that the frame 440 is cut from may have a uniform radial thickness 446 (as marked in FIG. 79). The frame 440 is cut from the body to result in the frame 440 having a uniform radial thickness 448 (marked in FIG. 75). The dimensions of the frame 440 may include the radial thickness 448 (marked in FIG. 75), and a length 450 (marked in FIG. 74), and a circumferential width 452 (marked in FIG. 74). The anchors 451 may each have a radial thickness 448 (marked in FIG. 75) and a circumferential width 452 (marked in FIG. 74).

In examples, a dimension of the frame 440 may desirably be reduced. Such a reduction may increase a flexibility of a portion of the frame 440 or provide other beneficial results. In examples, a dimension of one or more of the anchors 451 may be reduced with electropolishing. The reduction may increase a flexibility of the one or more anchors 451 or provide other beneficial results.

The dimension to be reduced may comprise a radial thickness 448 of the frame 440 or anchors 451. The radial thickness 448 may be reduced to increase flexibility of the portion of the frame 440 or anchors 451 or provide other beneficial results. The radial thickness may be difficult to reduce mechanically due to the manufacturing process of the frame 440. For example, the configuration of the frame 440 may be cut from material having a uniform thickness, and a cutting process alone may be difficult to reduce the radial thickness. A frame 440 as shown in FIG. 74, for example, may be cut to reduce a length 450 or circumferential width 452 of the frame 440, yet reducing the radial thickness may provide difficulties in a cutting process.

An electropolishing process may be utilized to reduce the dimension of the frame 440, which may comprise the radial thickness 448 of the frame 440. Electropolishing, for example, may comprise an electrochemical process that may remove material from a surface of a body. The electropolishing may occur in an electropolishing bath 454 that may include an electrolyte, with the body comprising an anode and a cathode being provided in the bath 454 to remove material from the body upon a current being provided. The electropolishing may reduce material in a dimension of the body, which may comprise a radial thickness of the body.

The electropolishing may occur at a variety of locations upon the frame 440 as desired. For example, referring to FIG. 74, one or more of the anchors 451 may be provided in the electropolishing bath 454 to remove material from the one or more anchors 451.

In examples, one or more of the anchors 451 may be inserted or dipped into the electropolishing bath 454 in a manner that may control the rate at which material is removed from the one or more anchors 451. For example, referring to FIG. 75, the one or more anchors 451 may be inserted into the bath 454 with a tip portion 456 inserted prior to an intermediate portion 458 of the anchor 451. The tip portion 456 may be inserted first such that the tip end 460 of the anchor 451 has been in the bath 454 longer than a more proximal portion of the anchor 451. Accordingly, a taper of the anchor 451 may result based on the amount of time that the anchor 451 has been within the bath 454, with the portion of the anchor 451 that has been in the bath 454 longer has a lesser thickness 448 than the portion of the anchor 451 that has been in the bath 454 for less time. The radial thickness of the one or more anchors 451 may be tapered with the electropolishing. The rate of insertion into the bath 454 and the time that the anchor 451 has spent in the bath may be controlled to provide a desired reduction of material and radial thickness 448 of the anchor 451.

Notably, other dimensions of the anchor 451 may be reduced in the bath 454. For example, the circumferential width 452 (marked in FIG. 74) may be reduced and a length may be reduced with the radial thickness 448 of the anchor 451.

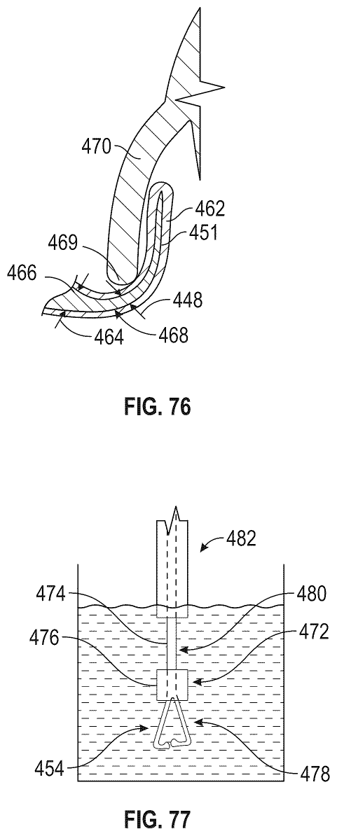

A reduced radial thickness 448 of the anchor 451 may increase a flexibility of the anchor 451. For example, referring to FIG. 76, a side cross sectional view of the anchor 451 is shown, with the anchor 451 having a cover 462 positioned thereon. The cover 462 may provide a cushion upon the anchor 451. The anchor 451 may be configured to extend over a distal tip of a native valve leaflet with the reduced radial thickness 448 of the anchor 451 increasing a flexibility of the anchor 451. The radial thickness 448 may be reduced relative to a radial thickness 464 of a proximal portion 466 of the anchor 451. In examples, the radial thickness 448 may be reduced at a bend portion 468 of the anchor 451 that may be positioned at a distal tip 469 of the native valve leaflet 470. The reduced radial thickness 448 at the bend portion 468 may increase a flexibility of the anchor 451 in directions radially inward and outward from the prosthetic valve. A reduced possibility of conduction disturbance may be provided by the anchor 451. Less fatigue strain may be provided and the anchor 451 may be more durable in examples.

In examples, the radial thickness of an anchor may increase and decrease along a length of an anchor 451. Such a result may occur in an electropolishing process. For example, referring to FIG. 77, a portion 472 of an anchor 474 may be covered with a material that insulates the portion 472 from an electropolishing process. A portion of a body may be covered to reduce the electropolishing of that portion of the body while other portions of the body are electropolished. As such, the portion 472 may lack a removal of material in an electropolishing process, or may have material removed at a lesser rate than other portions of the anchor 474. The covering may comprise an insulating body 476 in examples or may have another form.

The portion 472 may comprise a first intermediate portion 472 of the anchor 474, which may be positioned proximal of the tip portion 478 of the anchor 474. A second intermediate portion 480 of the anchor 474 may be proximal of the first intermediate portion and exposed to the bath 454 and accordingly reduction of material may occur at the second intermediate portion 480. A proximal portion 482 of the anchor 474 may be covered with an insulating body and thus may lack a removal of material in an electropolishing process, or may have material removed at a lesser rate than other portions of the anchor 474. The insulating bodies may be removed following the electropolishing process.

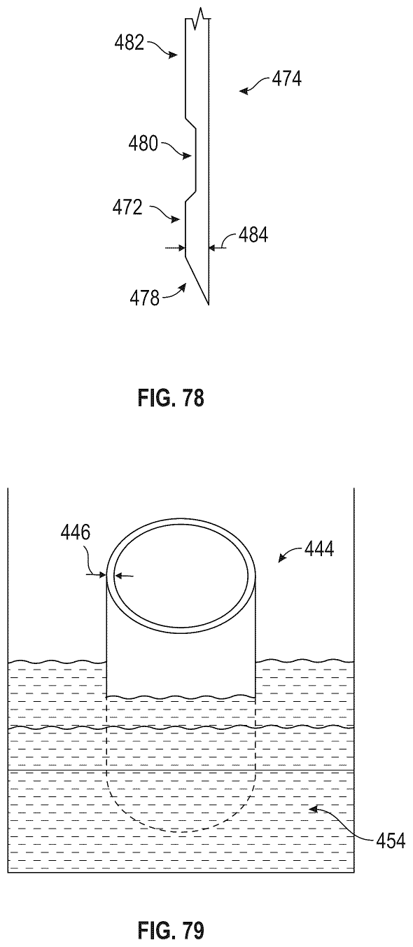

Referring to FIG. 78, the resulting anchor 474 may include a first intermediate portion 472 that may have a greater radial thickness 484 than the tip portion 478. The second intermediate portion 480 may have a lesser radial thickness 484 than the first intermediate portion 472. The second intermediate portion 480 may have a lesser radial thickness 484 than the proximal portion 482. In examples, the second intermediate portion 480 may correspond to a bend portion of the anchor 474, which may be positioned for the first intermediate portion 472 and the tip portion 478 to pivot about.

In examples, other portions of the frame 440 may undergo an electropolishing process to have a dimension of the frame 440 reduced. For example, the valve body 449 may have a dimension (e.g., radial thickness, circumferential width, or length) reduced in an electropolishing process. At least one of the plurality of struts of the frame 440 may have a dimension reduced with electropolishing. In examples, combinations of anchors and other portions of a frame 440 may be reduced in an electropolishing process.

In examples, a body, for example, a continuous or uncut cylinder 444 may have a dimension reduced in an electropolishing process. For example, referring to FIG. 79, a continuous or uncut cylinder 444 may be inserted into the bath 454. The continuous or uncut cylinder 444 may comprise a tube that is then cut to produce a frame as shown in FIG. 74 for example. In a configuration as shown in FIG. 79, the outer surface of the cylinder 444 may be exposed to the bath 454 such that the radial thickness 446 of the cylinder 444 may be reduced. In examples, the portions of the cylinder 444 that correspond to the anchors 451 or other portions of the frame 440 shown in FIG. 74 may be reduced in a desired manner. A method may include electropolishing at least a portion of a cylinder to reduce a dimension of the portion of the cylinder.

The cylinder 444 may then be cut following the electropolishing to produce a frame 440 as shown in FIG. 74, with the corresponding portions of the frame 440 having a reduced dimensionality (e.g., a reduced radial thickness of the anchors 451 or other portion of the frame 440 as desired). The cylinder 444 may be cut to include the one or more anchors for the prosthetic valve following the electropolishing. A prosthetic valve may be formed utilized the body, which may form the frame or other portions of the prosthetic valve.

In examples, other methods may be utilized to reduce a dimensionality of a portion of a prosthetic valve. For example, individual components may be electropolished to reduce dimensionality as desired. One anchor may have a greater reduction of dimensionality than another anchor, to produce a varied result as desired.

The features of FIGS. 74-79 may be utilized with any example of prosthetic valve disclosed herein.

In examples, a prosthetic valve may be provided that may be configured to be deployed to a heart valve of a patient's heart, with at least a portion of the prosthetic valve being configured to include one or more drugs for interaction with a patient's body.

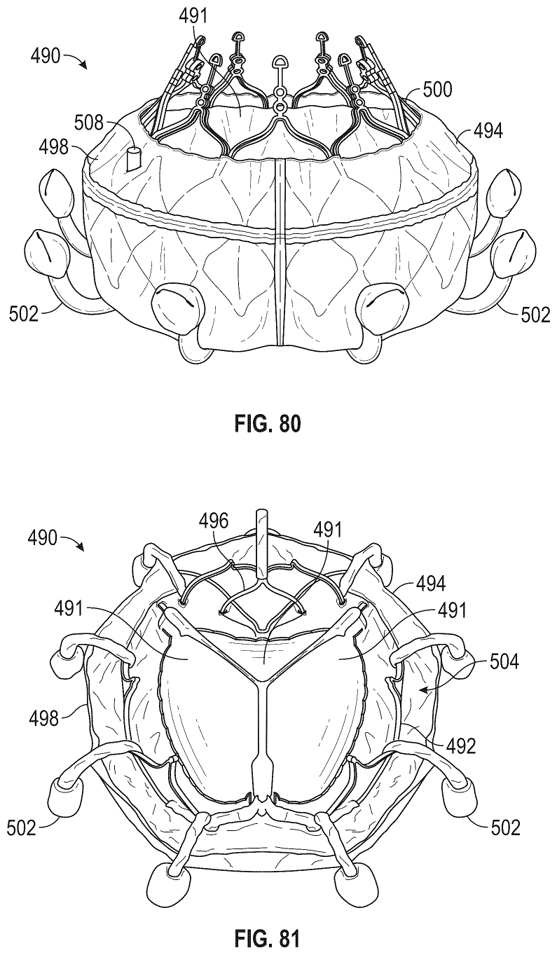

Referring to FIG. 80, a prosthetic valve may include a plurality of components, any of which may be provided with one or more drugs for interaction with a patient's body. For example, a prosthetic valve 490 may include a plurality of prosthetic valve leaflets 491, an inner valve body 492 (marked in FIG. 81), and may include a scaling body 494. The inner valve body 492 may include a frame 496 that may support the plurality of prosthetic valve leaflets 491.

In examples, the sealing body 494 may be configured to form a seal with at least a portion of a heart valve of a heart. The sealing body 494 may include a skirt 498 that may be configured to seal against a portion of a native heart valve. The sealing body 494 may include a frame 500 that may support the skirt 498. The frame 500 may comprise an outer frame in an example in which the inner valve body 492 comprises a frame 496.

In examples, one or more anchors 502 may extend radially outward from the prosthetic valve 490. The one or more anchors 502 may be configured to anchor the prosthetic valve 490 to a native heart valve. The one or more anchors 502 may be configured to curve proximally to anchor to a native heart valve leaflet or other portion of a native heart valve.

A prosthetic valve as disclosed herein may include a greater number or fewer number of components as desired. The configuration of the prosthetic valve utilized herein may be varied as desired.

One or more drugs may be included with the prosthetic valve in a variety of manners. For example, the one or more drugs may be coupled to the prosthetic valve. The one or more drugs, for example, may be embedded, impregnated, coated, or otherwise coupled to at least a portion of the prosthetic valve. A skirt or frame strut may be embedded, impregnated, coated, or otherwise coupled to one or more drugs. In examples, a pocket may be provided as a reservoir for one or more of the drugs. Other methods of including one or more drugs with the prosthetic valve may be utilized.

In examples, the one or more drugs may be included with the prosthetic valve to produce a localized effect. For example, the drugs may provide an interaction with the patient's body that may be local to the prosthetic valve. Such interactions may include reducing thrombosis formation on a portion of the prosthetic valve. Such interactions may include encouraging tissue ingrowth with a portion of the prosthetic valve. Such interactions may include reducing infection at the implantation site of the prosthetic valve.

One or more drugs may be utilized to reduce blood clot, reduce inflammation, and reduce body rejection of implants. Such drugs may be included in frames or skirts or other components of a prosthetic valve. Exemplary drugs that may be utilized may comprise Everolimus, Ridaforolimus, Umirolimus, Sirolimus, Amphilimus, Zotarolimus, or Everolimus. An antithrombosis drug may be utilized. Drugs configured to encourage tissue ingrowth may be utilized.

One or more drugs may be configured to treat a malady such as cancer or heart failure. Beta blockers or diuretics may be utilized. Exemplary drugs that may be utilized may comprise Bisoprolol, Metoprolol succinate, Carvedilol, and Carvedilol CR (Coreg CR) Toprol XL, among others. Exemplary drugs that may be utilized may comprise Furosemide, Bumetanide, Torsemide, Chlorothiazide, Amiloride, Hydrochlorothiazide or HCTZ, Indapamide, Metolazone, and Triamterene, among others.

Other forms of drugs may be utilized as desired.

The prosthetic valve may retain the one or more drugs and release the one or more drugs into the patient's body in examples. For example, the one or more drugs may be released into the bloodstream or to local tissue. The drugs may be bioresorbable. In examples, the one or more drugs may be retained on the prosthetic valve and produce the interaction with the patient's body upon blood or tissue contacting the one or more drugs.

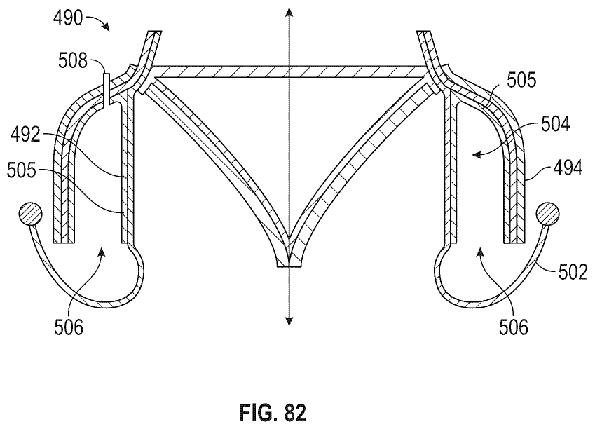

In examples, a pocket may be provided as a reservoir for one or more of the drugs. For example, referring to FIGS. 80-82, a pocket 504 may be provided between an inner valve body 492 and an outer valve body such as a scaling body 494. Referring to FIG. 82, the pocket 504 may be positioned in a space between the inner valve body 492 and the sealing body 494. The pocket 504 may be lined with a liner 505 that may be fluid impermeable in examples.

The pocket 504 may be configured to retain the one or more drugs therein. The one or more drugs may release from the pocket 504 via one or more openings 506, such as the opening 506 between the sealing body 494 and the inner valve body 492.

In examples, the pocket 504 may be prefilled with one or more drugs prior to deployment to an implantation site. Upon deployment of the prosthetic valve 490, the one or more drugs may be positioned within the pocket 504 and configured to interact with the patient's body.

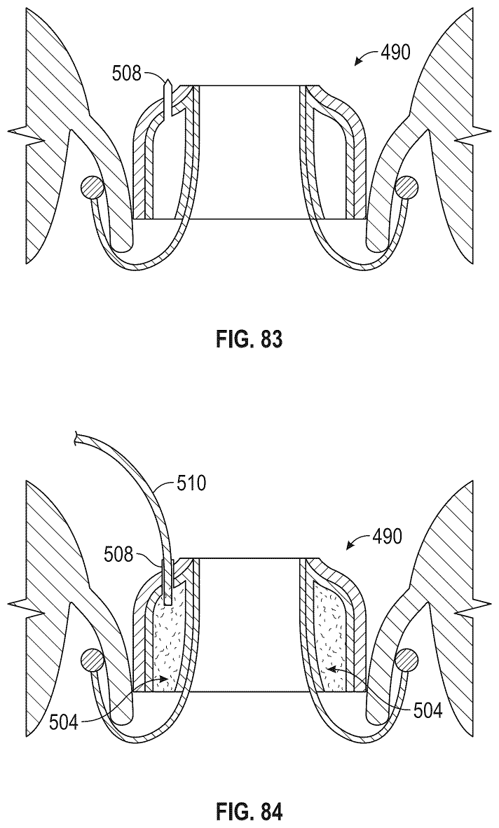

In examples, the pocket 504 may be filled or refilled in vivo via a port 508. The port 508 may be connected to the pocket 504 and configured to fill the pocket 504 with the one or more drugs. For example, referring to FIG. 83, the prosthetic valve 490 may be deployed to an implantation site. The port 508 may be accessible to a dispensing apparatus 510 (marked in FIG. 84) that may be inserted in vivo to fill the pocket 504. The dispensing apparatus 510 may comprise a fill catheter or other form of dispensing apparatus for the one or more drugs. The one or more drugs may be dispensed from the dispensing apparatus 510 through the port 508 to fill the pocket 504.

The dispensing apparatus 510 may fill the pocket 504 at the time of the implantation procedure for the prosthetic valve 490, or may fill the pocket 504 in a subsequent procedure. For example, following implantation of the prosthetic valve 490, the pocket 504 may be refilled with the one or more drugs. Such refilling may be periodic or may be based on a desired interaction of the drugs with the patient's body. For example, if the patient's heart or other part of the patient's body has a malady or prospectively has a malady, then the one or more drugs may fill the pocket 504 or may be interchanged with different drugs as desired.

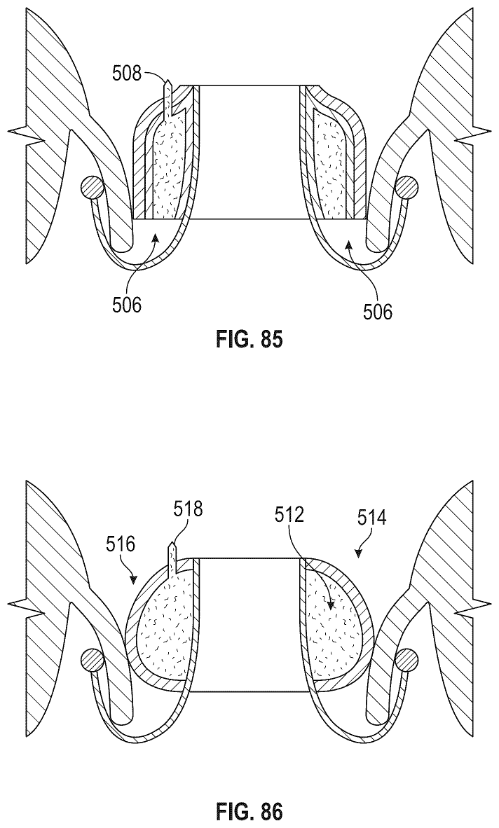

The dispensing apparatus 510 may be withdrawn from the prosthetic valve 490 following the pocket 504 being filled. Referring to FIG. 85, the port 508 may be configured to close and retain the one or more drugs within the pocket 504 or may be configured to release the drugs into the patient's body as desired. An opening 506 may remain open for the pocket 504 to allow the one or more drugs to release into the patient's body.

In examples, a pocket 512 may be provided that may be configured to inflate with the one or more drugs upon the pocket 512 being filled. For example, referring to FIG. 86, the prosthetic valve 514 may lack an outer frame or may lack rigidity for an outer surface 516 of the prosthetic valve 514. The outer surface 516 may be configured to be expandable in diameter upon the pocket 512 being filled with the one or more drugs. As such, a seal may be provided by the outer surface 516 against an interior surface of the heart valve due to the filling of the pocket 512. An amount of the drugs filling the pocket 512 may determine the diameter of the outer surface 516. The diameter of the outer surface 516 may be set by varying the amount of the drugs that fill the pocket 512. In examples, a port 518 may be utilized to fill the pocket 512.

The outer surface 516 of the prosthetic valve 514 may be configured to be permeable to the one or more drugs. As such, the one or more drugs may interact with the patient's body through the outer surface 516 of the prosthetic valve 514. In examples, other forms of release of the one or more drugs may be provided (e.g., one or more openings or via the port 518). In examples, the pocket 512 may be configured to be refilled with the one or more drugs via the port 518.

The one or more drugs may comprise a fluid, or may be in a solid configuration. For example, one or more beads or other solid shapes may be provided that comprise the drugs. The beads may be configured to fill a pocket as shown in FIG. 85 or 86 or may be otherwise utilized.

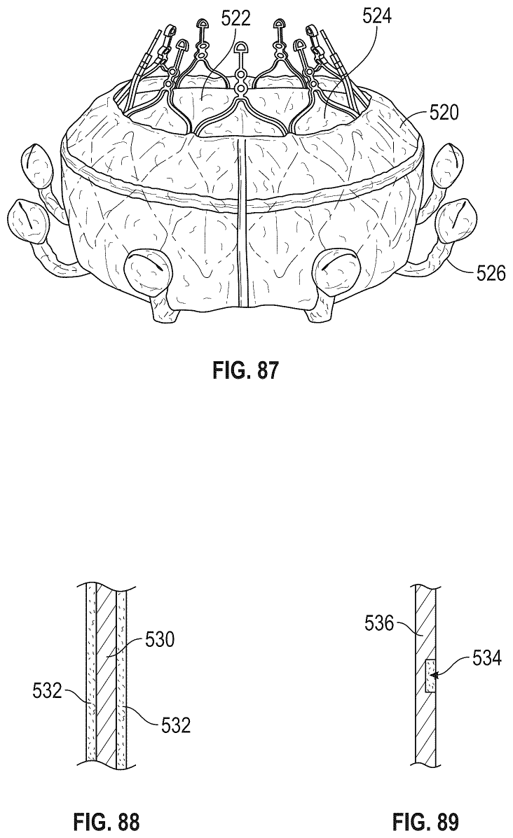

Other configurations of prosthetic valves may be utilized to include the one or more drugs. For example, referring to FIG. 87, a surface of one or more of an outer body or scaling body 520, an inner valve body 522, prosthetic valve leaflets 524, or anchors 526 may include the one or more drugs. At least a portion of a skirt may include one or more drugs. The skirt may comprise a sealing skirt for forming a seal with at least a portion of a heart valve. At least a portion of one or more anchors 526 may include the one or more drugs. At least a portion of one or more sutures of the prosthetic valve may include the one or more drugs. The one or more drugs may be embedded, impregnated, coated, or otherwise coupled to such components. An outer surface of one or more of such components may include one or more drugs.

In examples, different portions of a prosthetic valve may include different drugs to provide different interactions with a patient's body. For example, as shown in FIG. 87, an outer surface of the outer body or sealing body 520 may include a drug that encourages tissue ingrowth. Such a drug may improve a seal with a native implantation site. An inner valve body 522 or inner facing surface of such an inner valve body 522, may include a drug that reduces thrombosis, to reduce the possibility of clotting, pannus formation, or other adverse effects from forming around the prosthetic valve leaflets and the flow channel for the prosthetic valve leaflets. In examples, at least a portion of one of the prosthetic valve leaflets may include one or more drugs.

At least a portion of a frame may include one or more drugs. The frame may be embedded, impregnated, coated, or otherwise coupled to one or more drugs. For example, FIG. 88 illustrates a cross sectional view of a frame strut 530 with a coating 532 of a drug upon the frame strut 530. Any portion of a frame or anchor may include such a coating.

In examples, a portion of a frame may include one or more recesses 534 for receiving one or more drugs. FIG. 89, for example, illustrates a laser cut recess 534 in a strut 536 for receiving the one or more drugs. The drugs may interact with the patient's body by being released into the body or providing an interaction with a portion of the patient's body upon contact of the drugs with that portion of the body.

The features of FIGS. 80-89 may be utilized with any example of prosthetic valve disclosed herein.

The examples of prosthetic valves may be utilized in a mitral valve as disclosed herein, or may be utilized in other deployment locations such as a native tricuspid valve, or other deployment locations. The examples of prosthetic valves disclosed herein may be utilized for replacing a mitral valve, or may be utilized for replacing a tricuspid valve, or other forms of valves.

Various modifications of the examples disclosed herein may be provided. Features of examples may be modified, substituted, excluded, or combined across examples as desired. Combinations of features across examples may be provided as desired. Combinations of features may be provided across examples with other features of such examples being excluded if desired.

The various examples of sealing skirts disclosed herein may have a variety of forms, including cloth skirts, foam skirts, or braided skirts as desired. Various materials may be utilized as desired.

The implants disclosed herein may include prosthetic heart valves or other forms of implants, such as stents or filters, or diagnostic devices, among others. The implants may be expandable implants configured to move from a compressed or undeployed state to an expanded or deployed state. The implants may be compressible implants configured to be compressed inward to have a reduced outer profile and to move the implant to the compressed or undeployed state.

Various forms of delivery apparatuses may be utilized with the examples disclosed herein. The delivery apparatuses as disclosed herein may be utilized for aortic, mitral, tricuspid, and pulmonary replacement and repair as well. The delivery apparatuses may comprise delivery apparatuses for delivery of other forms of implants, such as stents or filters, or diagnostic devices, among others.

The implants and the systems disclosed herein may be used in transcatheter mitral or tricuspid implantation, as well as aortic valve implantation (TAVI) or replacement of other native heart valves (e.g., pulmonary valves). The delivery apparatuses and the systems disclosed herein may be utilized for transarterial access, including transfemoral access, to a patient's heart. The delivery apparatuses and systems may be utilized in transcatheter percutaneous procedures, including transarterial procedures, which may be transfemoral or transjugular. Transapical procedures, among others, may also be utilized. Other procedures may be utilized as desired.

In addition, the methods herein are not limited to the methods specifically described and may include methods of utilizing the systems and apparatuses disclosed herein. The steps of the methods may be modified, excluded, or added to, with systems, apparatuses, and methods disclosed herein. The examples disclosed herein may comprise systems for implantation within a human body in examples.

For purposes of this description, certain aspects, advantages, and novel features of the examples of this disclosure are described herein. The disclosed methods, apparatuses, and systems should not be construed as limiting in any way. Instead, the present disclosure is directed toward all novel and nonobvious features and aspects of the various disclosed examples, along and in various combinations and sub-combinations with one another. The methods, apparatuses, and systems are not limited to any specific aspect or feature or combination thereof, nor do the disclosed examples require that any one or more specific advantages be present or problems be solved. Features, elements, or combinations of one example can be combined into other examples herein.
Example 1: A prosthetic valve for implantation within a heart valve, the prosthetic valve comprising: a plurality of prosthetic valve leaflets; a valve frame configured to support the plurality of prosthetic valve leaflets; and a plurality of elongate anchor arms each having a first end portion coupled to the valve frame and each configured to extend radially outward from the valve frame to a second end portion that is configured to anchor to an interior surface of the heart valve.
Example 2: The prosthetic valve of any example herein, in particular Example 1, wherein each of the plurality of elongate anchor arms hooks.
Example 3: The prosthetic valve of any example herein, in particular Example 1 or Example 2, wherein the valve frame includes a proximal end portion and a distal end portion, and the plurality of elongate anchor arms extend radially outward from the distal end portion of the valve frame.
Example 4: The prosthetic valve of any example herein, in particular Example 3, wherein each of the first end portions of the plurality of elongate anchor arms are coupled to the distal end portion of the valve frame.
Example 5: The prosthetic valve of any example herein, in particular Examples 1-4, wherein each of the plurality of elongate anchor arms includes a bend portion between the first end portion and the second end portion.
Example 6: The prosthetic valve of any example herein, in particular Example 5, wherein the first end portion is configured to extend in a first direction, and the bend portion is configured to direct the second end portion to extend in a second direction that is opposed to the first direction.
Example 7: The prosthetic valve of any example herein, in particular Example 6, wherein the first direction is a distal direction and the second direction is a proximal direction.
Example 8: The prosthetic valve of any example herein, in particular Examples 5-7, wherein the bend portion has a convex shape directed distally.
Example 9: The prosthetic valve of any example herein, in particular Examples 5-8, wherein the bend portion includes one or more cuts for enhancing flexibility of the respective one of the elongate anchor arms.
Example 10: The prosthetic valve of any example herein, in particular Examples 1-9, wherein the second end portions extend straight.
Example 11: The prosthetic valve of any example herein, in particular Examples 1-10, wherein the plurality of elongate anchor arms are configured to move from an undeployed configuration to a deployed configuration, the plurality of elongate anchor arms extending longitudinally from the first end portion to the second end portion in the undeployed configuration, the plurality of elongate anchor arms deflecting radially outward from the undeployed configuration to the deployed configuration.
Example 12: The prosthetic valve of any example herein, in particular Example 11, wherein the valve frame includes a proximal end portion and a distal end portion, and each of the first end portions of the plurality of elongate anchor arms are coupled to the distal end portion of the valve frame in the undeployed configuration.
Example 13: The prosthetic valve of any example herein, in particular Example 11 or Example 12, wherein each of the plurality of elongate anchor arms includes a bend portion between the first end portion and the second end portion, and the plurality of elongate anchor arms are configured to rotate about the bend portion to move from the undeployed configuration to the deployed configuration.
Example 14: The prosthetic valve of any example herein, in particular Example 13, wherein each of the plurality of elongate anchor arms extends distally in the undeployed configuration and is configured to rotate about the bend portion to extend proximally in the deployed configuration.
Example 15: The prosthetic valve of any example herein, in particular Examples 1-14, wherein each of the plurality of elongate anchor arms are circumferentially spaced from each other.
Example 16: The prosthetic valve of any example herein, in particular Examples 1-15, wherein the plurality of elongate anchors arms is configured to form a ring around the valve frame, with an annular gap positioned between the ring and the valve frame.
Example 17: The prosthetic valve of any example herein, in particular Examples 1-16, wherein a sealing skirt extends along the plurality of elongate anchor arms.
Example 18: The prosthetic valve of any example herein, in particular Examples 1-17, wherein each of the plurality of elongate anchor arms includes one or more anchoring protrusions for engaging the interior surface of the heart valve.
Example 19: The prosthetic valve of any example herein, in particular Example 18, wherein at least one of the one or more anchoring protrusions is angled proximally.
Example 20: The prosthetic valve of any example herein, in particular Example 18 or Example 19, further comprising one or more covering bodies for covering at least one of the one or more anchoring protrusions.
Example 21: The prosthetic valve of any example herein, in particular Example 20, wherein the one or more covering bodies include a plurality of sheaths each extending along a respective one of the plurality of elongate anchor arms.
Example 22: The prosthetic valve of any example herein, in particular Example 20 or Example 21, wherein the one or more covering bodies include a skirt that each of the plurality of elongate anchor arms are positioned within.
Example 23: The prosthetic valve of any example herein, in particular Example 22, wherein the one or more anchoring protrusions are configured to pass through the skirt.
Example 24: The prosthetic valve of any example herein, in particular Example 22 or Example 23, wherein the plurality of elongate anchor arms is configured to rotate within the skirt to move from an undeployed configuration to a deployed configuration.
Example 25: The prosthetic valve of any example herein, in particular Examples 1-24, wherein the valve frame includes a proximal end portion and a distal end portion, and each of the first end portions of the plurality of elongate anchor arms are coupled to the proximal end portion of the valve frame, and each of the plurality of elongate anchor arms extend distally from the first end portion to the second end portion of the respective elongate anchor arm.
Example 26: The prosthetic valve of any example herein, in particular Example 25, further comprising a skirt extending around the second end portions of the plurality of elongate anchor arms.
Example 27: The prosthetic valve of any example herein, in particular Example 26, further comprising a plurality of arms extending radially outward from the distal end portion of the valve frame and supporting the skirt, and wherein each of the second end portions include one or more anchoring protrusions configured to pass through the skirt to engage the interior surface of the heart valve.
Example 28: The prosthetic valve of any example herein, in particular Examples 1-27, wherein the plurality of elongate anchor arms comprise a first plurality of elongate anchor arms, and the prosthetic valve further comprises a second plurality of elongate anchor arms each configured to hook over a distal tip of a leaflet of the heart valve.
Example 29: The prosthetic valve of any example herein, in particular Example 28, wherein at least one of the second plurality of elongate anchor arms is positioned between two of the first plurality of elongate anchor arms.
Example 30: The prosthetic valve of any example herein, in particular Examples 1-29, wherein the prosthetic valve is configured for implantation within a mitral heart valve or a tricuspid heart valve.
Example 31: A method comprising: deploying a prosthetic valve within a heart valve, the prosthetic valve including: a plurality of prosthetic valve leaflets, a valve frame configured to support the plurality of prosthetic valve leaflets, and a plurality of elongate anchor arms each having a first end portion coupled to the valve frame and each configured to extend radially outward from the valve frame to a second end portion that is configured to anchor to an interior surface of the heart valve.
Example 32: The method of any example herein, in particular Example 31, wherein each of the plurality of elongate anchor arms hooks.
Example 33: The method of any example herein, in particular Example 31 or Example 32, wherein the plurality of elongate anchor arms are configured to move from an undeployed configuration to a deployed configuration, the plurality of elongate anchor arms extending longitudinally from the first end portion to the second end portion in the undeployed configuration, the plurality of elongate anchor arms deflecting radially outward from the undeployed configuration to the deployed configuration.
Example 34: The method of any example herein, in particular Example 33, wherein the valve frame includes a proximal end portion and a distal end portion, and each of the first end portions of the plurality of elongate anchor arms are coupled to the distal end portion of the valve frame in the undeployed configuration.
Example 35: The method of any example herein, in particular Example 33 or Example 34, wherein each of the plurality of elongate anchor arms includes a bend portion between the first end portion and the second end portion, and the plurality of elongate anchor arms are configured to rotate about the bend portion to move from the undeployed configuration to the deployed configuration.
Example 36: The method of any example herein, in particular Example 35, wherein each of the plurality of elongate anchor arms extends distally in the undeployed configuration and is configured to rotate about the bend portion to extend proximally in the deployed configuration.
Example 37: The method of any example herein, in particular Examples 31-36, wherein the plurality of elongate anchors arms is configured to form a ring around the valve frame, with an annular gap positioned between the ring and the valve frame.
Example 38: The method of any example herein, in particular Examples 31-37, wherein a sealing skirt extends along the plurality of elongate anchor arms.
Example 39: The method of any example herein, in particular Examples 31-38, wherein each of the plurality of elongate anchor arms include one or more anchoring protrusions for engaging the interior surface of the heart valve.
Example 40: The method of any example herein, in particular Example 39, further comprising one or more covering bodies for covering at least one of the one or more anchoring protrusions.
Example 41: The method of any example herein, in particular Example 40, wherein the one or more covering bodies include a plurality of sheaths each extending along a respective one of the plurality of elongate anchor arms.
Example 42: The method of any example herein, in particular Example 40 or Example 41, wherein the one or more covering bodies include a skirt that each of the plurality of elongate anchor arms are positioned within.
Example 43: The method of any example herein, in particular Example 42, wherein the one or more anchoring protrusions are configured to pass through the skirt.
Example 44: The method of any example herein, in particular Example 42 or Example 43, wherein the plurality of elongate anchor arms is configured to rotate within the skirt to move from an undeployed configuration to a deployed configuration.
Example 45: The method of any example herein, in particular Examples 31-44, wherein the valve frame includes a proximal end portion and a distal end portion, and each of the first end portions of the plurality of elongate anchor arms are coupled to the proximal end portion of the valve frame, and each of the plurality of elongate anchor arms extend distally from the first end portion to the second end portion of the respective elongate anchor arm.
Example 46: The method of any example herein, in particular Example 45, further comprising a skirt extending around the second end portions of the plurality of elongate anchor arms.
Example 47: The method of any example herein, in particular Example 46, further comprising a plurality of arms extending radially outward from the distal end portion of the valve frame and supporting the skirt, and wherein each of the second end portions include one or more anchoring protrusions configured to pass through the skirt to engage the interior surface of the heart valve.
Example 48: The method of any example herein, in particular Examples 31-47, wherein the plurality of elongate anchor arms comprise a first plurality of elongate anchor arms, and the prosthetic valve further comprises a second plurality of elongate anchor arms each configured to hook over a distal tip of a leaflet of the heart valve.
Example 49: The method of any example herein, in particular Example 48, wherein at least one of the second plurality of elongate anchor arms is positioned between two of the first plurality of elongate anchor arms.
Example 50: The method of any example herein, in particular Examples 31-49, wherein the heart valve is a mitral heart valve or a tricuspid heart valve.
Example 51: A prosthetic valve for implantation within a heart valve, the prosthetic valve comprising: a plurality of prosthetic valve leaflets; a valve frame configured to support the plurality of prosthetic valve leaflets; a plurality of anchors each configured to extend radially outward from the valve frame and be positioned radially outward of the heart valve to anchor to the heart valve; and a plurality of sealing bodies configured to extend radially outward from the valve frame, each positioned between two of the plurality of anchors and configured to be positioned radially inward of the heart valve to seal fluid flow.
Example 52: The prosthetic valve of any example herein, in particular Example 51, wherein each of the plurality of sealing bodies comprises a protrusion configured to extend radially outward from the valve frame.
Example 53: The prosthetic valve of any example herein, in particular Example 51 or Example 52, wherein each of the plurality of sealing bodies comprises a scaling strut.
Example 54: The prosthetic valve of any example herein, in particular Examples 51-53, wherein the plurality of anchors alternates with the plurality of sealing bodies circumferentially about the valve frame.
Example 55: The prosthetic valve of any example herein, in particular Examples 51-54, wherein a sealing skirt extends along the plurality of sealing bodies.
Example 56: The prosthetic valve of any example herein, in particular Example 55, wherein the sealing skirt is configured to form a disk extending radially outward from the valve frame.
Example 57: The prosthetic valve of any example herein, in particular Examples 51-56, wherein the plurality of anchors and the plurality of sealing bodies are configured to clamp a portion of the heart valve between the plurality of anchors and the plurality of sealing bodies.
Example 58: The prosthetic valve of any example herein, in particular Examples 51-57, further comprising one or more tethers configured to couple the plurality of sealing bodies to the valve frame and configured to vary a height of the plurality of sealing bodies.
Example 59: The prosthetic valve of any example herein, in particular Example 58, wherein a height of the valve frame is configured to vary, and the one or more tethers are configured to reduce a height of the plurality of sealing bodies upon the height of the valve frame decreasing.
Example 60: The prosthetic valve of any example herein, in particular Examples 51-59, wherein the plurality of anchors comprises a plurality of elongate anchor arms.
Example 61: The prosthetic valve of any example herein, in particular Example 60, wherein the valve frame includes a proximal end portion and a distal end portion, and the plurality of elongate anchor arms and the plurality of sealing bodies each extend radially outward from the distal end portion of the valve frame.
Example 62: The prosthetic valve of any example herein, in particular Example 61, wherein first end portions of the plurality of elongate anchor arms and first end portions of the plurality of sealing bodies are each coupled to the distal end portion of the valve frame.
Example 63: The prosthetic valve of any example herein, in particular Examples 60-62, wherein each of the plurality of elongate anchor arms includes a bend portion.
Example 64: The prosthetic valve of any example herein, in particular Example 63, wherein the first end portions of the plurality of elongate anchor arms extend in a first direction, and the bend portion is configured to direct a second end portion of the plurality of elongate anchor arms to extend in a second direction that is opposed to the first direction.
Example 65: The prosthetic valve of any example herein, in particular Example 63 or Example 64, wherein the bend portion has a convex shape directed distally.
Example 66: The prosthetic valve of any example herein, in particular Examples 60-65, wherein the plurality of elongate anchor arms are configured to move from an undeployed configuration to a deployed configuration, the plurality of elongate anchor arms having a first end portion and a second end portion, and the plurality of elongate anchor arms extend longitudinally from the first end portion to the second end portion in the undeployed configuration, the plurality of elongate anchor arms deflecting radially outward from the undeployed configuration to the deployed configuration.
Example 67: The prosthetic valve of any example herein, in particular Example 66, wherein the valve frame includes a proximal end portion and a distal end portion, and each of the first end portions of the plurality of elongate anchor arms are coupled to the distal end portion of the valve frame in the undeployed configuration.
Example 68: The prosthetic valve of any example herein, in particular Example 66 or Example 67, wherein each of the plurality of elongate anchor arms includes a bend portion between the first end portion and the second end portion, and the plurality of elongate anchor arms are configured to rotate about the bend portion to move from the undeployed configuration to the deployed configuration.
Example 69: The prosthetic valve of any example herein, in particular Example 68, wherein each of the plurality of elongate anchor arms extends distally in the undeployed configuration and is configured to rotate about the bend portion to extend proximally in the deployed configuration.
Example 70: The prosthetic valve of any example herein, in particular Examples 51-69, wherein the prosthetic valve is configured for implantation within a mitral heart valve or a tricuspid heart valve.
Example 71: A method comprising: deploying a prosthetic valve within a heart valve, the prosthetic valve including: a plurality of prosthetic valve leaflets; a valve frame configured to support the plurality of prosthetic valve leaflets; a plurality of anchors each configured to extend radially outward from the valve frame and be positioned radially outward of the heart valve to anchor to the heart valve, and a plurality of sealing bodies configured to extend radially outward from the valve frame, each positioned between two of the plurality of anchors and configured to be positioned radially inward of the heart valve to seal fluid flow.
Example 72: The method of any example herein, in particular Example 71, wherein each of the plurality of sealing bodies comprises a protrusion configured to extend radially outward from the valve frame.
Example 73: The method of any example herein, in particular Example 71 or Example 72, wherein each of the plurality of sealing bodies comprises a sealing strut.
Example 74: The method of any example herein, in particular Examples 71-73, wherein the plurality of anchors alternates with the plurality of sealing bodies circumferentially about the valve frame.
Example 75: The method of any example herein, in particular Examples 71-74, wherein a sealing skirt extends along the plurality of sealing bodies.
Example 76: The method of any example herein, in particular Example 75, wherein the sealing skirt is configured to form a disk extending radially outward from the valve frame.
Example 77: The method of any example herein, in particular Examples 71-76, wherein the plurality of anchors and the plurality of sealing bodies are configured to clamp a portion of the heart valve between the plurality of anchors and the plurality of sealing bodies.
Example 78: The method of any example herein, in particular Examples 71-77, wherein the valve frame includes a proximal end portion and a distal end portion, and the plurality of anchors and the plurality of sealing bodies each extend radially outward from the distal end portion of the valve frame.
Example 79: The method of any example herein, in particular Example 78, wherein first end portions of the plurality of anchors and first end portions of the plurality of sealing bodies are each coupled to the distal end portion of the valve frame.
Example 80: The method of any example herein, in particular Examples 71-79, wherein the prosthetic valve is configured for implantation within a mitral heart valve or a tricuspid heart valve.
Example 81: A prosthetic valve for implantation within a heart valve, the prosthetic valve comprising: a plurality of prosthetic valve leaflets; and an anchoring body coupled to the plurality of prosthetic valve leaflets and configured to anchor to the heart valve, the anchoring body including: a proximal frame and a distal frame and configured to form an annular recess between the proximal frame and the distal frame for receiving a portion of the heart valve, the proximal frame extending radially outward from a proximal end portion to form a first shoulder positioned proximal of the annular recess, and the distal frame extending radially outward from a distal end portion to form a second shoulder positioned distal of the annular recess.
Example 82: The prosthetic valve of any example herein, in particular Example 81, wherein the proximal frame includes a distal end portion and the distal frame includes a proximal end portion coupled to the distal end portion of the proximal frame.
Example 83: The prosthetic valve of any example herein, in particular Example 81 or Example 82, wherein the proximal frame includes a proximal end extending axially, and the distal frame includes a distal end extending axially.
Example 84: The prosthetic valve of any example herein, in particular Examples 81-83, wherein the anchoring body is positioned radially outward of an inner frame configured to support the plurality of prosthetic valve leaflets.
Example 85: The prosthetic valve of any example herein, in particular Example 84, wherein the inner frame has a cylindrical shape.
Example 86: The prosthetic valve of any example herein, in particular Example 84 or Example 85, wherein the proximal frame is configured to form a space between the first shoulder and the inner frame, and the distal frame is configured to form a space between the second shoulder and the inner frame.
Example 87: The prosthetic valve of any example herein, in particular Examples 84-86, wherein the proximal frame includes a distal end portion coupled to the inner frame, and the distal frame includes a proximal end portion coupled to the inner frame.
Example 88: The prosthetic valve of any example herein, in particular Examples 84-87, wherein the proximal frame includes a proximal end spaced from the inner frame, and the distal frame includes a distal end spaced from the inner frame.
Example 89: The prosthetic valve of any example herein, in particular Examples 84-86, wherein the proximal end portion of the proximal frame is coupled to the inner frame, and the distal end portion of the distal frame is coupled to the inner frame.
Example 90: The prosthetic valve of any example herein, in particular Examples 84-86 or 89, wherein a distal end of the proximal frame is spaced from the inner frame, and a proximal end of the distal frame is spaced from the inner frame.
Example 91: The prosthetic valve of any example herein, in particular Example 90, wherein the distal end of the proximal frame is deflectable in a direction radially inward and the proximal end of the distal frame is deflectable in a direction radially inward.
Example 92: The prosthetic valve of any example herein, in particular Examples 81-91, wherein the proximal frame is configured to move from an undeployed configuration to a deployed configuration and the distal frame is configured to move from an undeployed configuration to a deployed configuration independent from the proximal frame.
Example 93: The prosthetic valve of any example herein, in particular Examples 81-92, wherein the proximal frame includes a plurality of struts separated by openings and the distal frame includes a plurality of struts separated by openings.
Example 94: The prosthetic valve of any example herein, in particular Examples 81-93, wherein the proximal frame is configured to elongate upon being radially compressed inward and the distal frame is configured to elongate upon being radially compressed inward.
Example 95: The prosthetic valve of any example herein, in particular Examples 81-94, wherein the anchoring body is configured to clamp the portion of the heart valve between the proximal frame and the distal frame.
Example 96: The prosthetic valve of any example herein, in particular Examples 81-95, wherein the proximal frame is configured to resist distal movement of the prosthetic valve relative to the heart valve, and the distal frame is configured to resist proximal movement of the prosthetic valve relative to the heart valve.
Example 97: The prosthetic valve of any example herein, in particular Examples 81-96, further comprising a sealing skirt extending along one or more of the proximal frame or the distal frame.
Example 98: The prosthetic valve of any example herein, in particular Example 97, wherein at least a portion of the sealing skirt is configured to be positioned within the annular recess.
Example 99: The prosthetic valve of any example herein, in particular Example 97 or Example 98, wherein the sealing skirt extends from the proximal end portion of the proximal frame to the distal end portion of the distal frame.
Example 100: The prosthetic valve of any example herein, in particular Examples 81-99, wherein one or more of the proximal frame or the distal frame includes a flexible member configured to allow a respective one of the proximal frame or the distal frame to deflect.
Example 101: The prosthetic valve of any example herein, in particular Example 100, wherein the flexible member comprises an undulation in the respective one of the proximal frame or the distal frame.
Example 102: The prosthetic valve of any example herein, in particular Example 100 or Example 101, wherein the first shoulder includes the flexible member or the second shoulder includes the flexible member.
Example 103: The prosthetic valve of any example herein, in particular Examples 100-102, wherein the proximal frame includes an elongate arm extending distally from the flexible member or the distal frame includes an elongate arm extending proximally from the flexible member.
Example 104: The prosthetic valve of any example herein, in particular Examples 81-103, wherein the anchoring body is positioned radially outward of an inner frame configured to support the plurality of prosthetic valve leaflets, and the anchoring body is self-expanding and the inner frame is configured to be expanded with an inflatable body.
Example 105: The prosthetic valve of any example herein, in particular Examples 81-104, wherein the prosthetic valve is configured for implantation within a mitral heart valve or a tricuspid heart valve.
Example 106: A method comprising: deploying a prosthetic valve within a heart valve, the prosthetic valve including: a plurality of prosthetic valve leaflets, and an anchoring body coupled to the plurality of prosthetic valve leaflets and configured to anchor to the heart valve, the anchoring body including: a proximal frame and a distal frame and configured to form an annular recess between the proximal frame and the distal frame for receiving a portion of the heart valve, the proximal frame extending radially outward from a proximal end portion to form a first shoulder positioned proximal of the annular recess, and the distal frame extending radially outward from a distal end portion to form a second shoulder positioned distal of the annular recess.
Example 107: The method of any example herein, in particular Example 106, further comprising deploying the distal frame independently from the proximal frame.
Example 108: The method of any example herein, in particular Example 107, further comprising recapturing one or more of the distal frame or the proximal frame.
Example 109: The method of any example herein, in particular Examples 106-108, further comprising: advancing a distal portion of a capsule of a delivery apparatus relative to the distal frame to deploy the distal frame; and retracting a proximal portion of the capsule of the delivery apparatus relative to the proximal frame to deploy the proximal frame.
Example 110: The method of any example herein, in particular Examples 106-109, further comprising positioning the portion of the heart valve within the annular recess.
Example 111: The method of any example herein, in particular Examples 106-110, wherein the anchoring body is positioned radially outward of an inner frame configured to support the plurality of prosthetic valve leaflets.
Example 112: The method of any example herein, in particular Example 111, wherein the proximal frame is configured to form a space between the first shoulder and the inner frame, and the distal frame is configured to form a space between the second shoulder and the inner frame.
Example 113: The method of any example herein, in particular Examples 106-112, wherein the proximal frame is configured to resist distal movement of the prosthetic valve relative to the heart valve, and the distal frame is configured to resist proximal movement of the prosthetic valve relative to the heart valve.
Example 114: The method of any example herein, in particular Examples 106-113, further comprising a scaling skirt extending along one or more of the proximal frame or the distal frame.
Example 115: The method of any example herein, in particular Examples 106-114, wherein one or more of the proximal frame or the distal frame includes a flexible member configured to allow a respective one of the proximal frame or the distal frame to deflect.
Example 116: The method of any example herein, in particular Example 115, wherein the flexible member comprises an undulation in the respective one of the proximal frame or the distal frame.
Example 117: The method of any example herein, in particular Example 115 or Example 116, wherein the first shoulder includes the flexible member or the second shoulder includes the flexible member.
Example 118: The method of any example herein, in particular Examples 115-117, wherein the proximal frame includes an elongate arm extending distally from the flexible member or the distal frame includes an elongate arm extending proximally from the flexible member.
Example 119: The method of any example herein, in particular Examples 106-118, wherein the anchoring body is positioned radially outward of an inner frame configured to support the plurality of prosthetic valve leaflets, and the method further comprises: allowing the anchoring body to self-expand; and expanding the inner frame with an inflatable body.
Example 120: The method of any example herein, in particular Examples 106-119, wherein the prosthetic valve is configured for implantation within a mitral heart valve or a tricuspid heart valve.
Example 121: A prosthetic valve for implantation within a heart valve, the prosthetic valve comprising: a plurality of prosthetic valve leaflets; an inner frame supporting the plurality of prosthetic valve leaflets and including a plurality of anchors for anchoring the prosthetic valve to the heart valve; an outer frame configured to be positioned radially outward of the inner frame; and one or more tethers coupling the outer frame to the inner frame and configured to impede movement of the outer frame radially inward to reduce interlacing of the outer frame with the inner frame.
Example 122: The prosthetic valve of any example herein, in particular Example 121, wherein each of the one or more tethers includes a first end portion coupled to the outer frame and a second end portion coupled to the inner frame.
Example 123: The prosthetic valve of any example herein, in particular Example 122, wherein the second end portion is coupled to one or more of the plurality of anchors.
Example 124: The prosthetic valve of any example herein, in particular Example 123, wherein the second end portion is coupled to a bend portion of the one or more of the plurality of anchors.
Example 125: The prosthetic valve of any example herein, in particular Examples 122-124, wherein the first end portion is coupled to one or more apices of the outer frame.
Example 126: The prosthetic valve of any example herein, in particular Examples 121-125, wherein the one or more tethers extend circumferentially about the inner frame and alternate between a coupling to the inner frame and a coupling to the outer frame.
Example 127: The prosthetic valve of any example herein, in particular Examples 121-126, wherein the inner frame includes a plurality of struts separated by openings, and the one or more tethers are configured to reduce the outer frame from passing into one or more of the openings.
Example 128: The prosthetic valve of any example herein, in particular Examples 121-127, wherein the one or more tethers are configured to reduce twisting of the outer frame in a circumferential direction.
Example 129: The prosthetic valve of any example herein, in particular Examples 121-128, further comprising a sealing skirt coupled to the outer frame.
Example 130: The prosthetic valve of any example herein, in particular Examples 121-129, wherein the prosthetic valve is configured for implantation within a mitral heart valve or a tricuspid heart valve.
Example 131: A method comprising: deploying a prosthetic valve within a heart valve, the prosthetic valve including: a plurality of prosthetic valve leaflets; an inner frame supporting the plurality of prosthetic valve leaflets and including a plurality of anchors for anchoring the prosthetic valve to the heart valve; an outer frame configured to be positioned radially outward of the inner frame; and one or more tethers coupling the outer frame to the inner frame and configured to impede movement of the outer frame radially inward to reduce interlacing of the outer frame with the inner frame.
Example 132: The method of any example herein, in particular Example 131, wherein each of the one or more tethers includes a first end portion coupled to the outer frame and a second end portion coupled to the inner frame.
Example 133: The method of any example herein, in particular Example 132, wherein the second end portion is coupled to one or more of the plurality of anchors.
Example 134: The method of any example herein, in particular Example 133, wherein the second end portion is coupled to a bend portion of the one or more of the plurality of anchors.
Example 135: The method of any example herein, in particular Examples 132-134, wherein the first end portion is coupled to one or more apices of the outer frame.
Example 136: The method of any example herein, in particular Examples 131-135, wherein the one or more tethers extend circumferentially about the inner frame and alternate between a coupling to the inner frame and a coupling to the outer frame.
Example 137: The method of any example herein, in particular Examples 131-136, wherein the inner frame includes a plurality of struts separated by openings, and the one or more tethers are configured to reduce the outer frame from passing into one or more of the openings.
Example 138: The method of any example herein, in particular Examples 131-137, wherein the one or more tethers are configured to reduce twisting of the outer frame in a circumferential direction.
Example 139: The method of any example herein, in particular Examples 131-138, further comprising a scaling skirt coupled to the outer frame.
Example 140: The method of any example herein, in particular Examples 131-139, wherein the prosthetic valve is configured for implantation within a mitral heart valve or a tricuspid heart valve.
Example 141: A method comprising: compressing a prosthetic valve radially inward to crimp the prosthetic valve, the prosthetic valve including: a plurality of prosthetic valve leaflets, an inner frame supporting the plurality of prosthetic valve leaflets and including a plurality of anchors for anchoring the prosthetic valve to the heart valve, an outer frame configured to be positioned radially outward of the inner frame, and one or more tethers coupling the outer frame to the inner frame and configured to impede movement of the outer frame radially inward to reduce interlacing of the outer frame with the inner frame during the crimping of the prosthetic valve.
Example 142: The method of any example herein, in particular Example 141, further comprising clongating the outer frame and the inner frame while compressing the prosthetic valve.
Example 143: The method of any example herein, in particular Example 142, further comprising moving the plurality of anchors to a longitudinal configuration while compressing the prosthetic valve.
Example 144: The method of any example herein, in particular Examples 141-143, wherein each of the one or more tethers includes a first end portion coupled to the outer frame and a second end portion coupled to the inner frame.
Example 145: The method of any example herein, in particular Example 144, wherein the second end portion is coupled to one or more of the plurality of anchors.
Example 146: The method of any example herein, in particular Example 145, wherein the second end portion is coupled to a bend portion of the one or more of the plurality of anchors.
Example 147: The method of any example herein, in particular Examples 144-146, wherein the first end portion is coupled to one or more apices of the outer frame.
Example 148: The method of any example herein, in particular Examples 141-147, wherein the one or more tethers extend circumferentially about the inner frame and alternate between a coupling to the inner frame and a coupling to the outer frame.
Example 149: The method of any example herein, in particular Examples 141-148, wherein the inner frame includes a plurality of struts separated by openings, and the one or more tethers are configured to reduce the outer frame from passing into one or more of the openings.
Example 150: The method of any example herein, in particular Examples 141-149, wherein the one or more tethers are configured to reduce twisting of the outer frame in a circumferential direction.
Example 151: A prosthetic valve for implantation within a heart valve, the prosthetic valve comprising: a plurality of prosthetic valve leaflets; an inner frame supporting the plurality of prosthetic valve leaflets; and an outer frame configured to be positioned radially outward of the inner frame and configured to be assembled to the inner frame within a patient's body.
Example 152: The prosthetic valve of any example herein, in particular Example 151, further comprising one or more tethers extending between the inner frame and the outer frame and configured to draw the outer frame to the inner frame within the patient's body.
Example 153: The prosthetic valve of any example herein, in particular Example 152, further comprising one or more locks configured to couple to the one or more tethers to secure the inner frame to the outer frame within the patient's body.
Example 154: The prosthetic valve of any example herein, in particular Examples 151-153, further comprising one or more engagement bodies on one or more of the inner frame or the outer frame to secure the inner frame to the outer frame within the patient's body.
Example 155: The prosthetic valve of any example herein, in particular Examples 151-154, wherein: the inner frame is configured to expand from an undeployed configuration to a deployed configuration, the inner frame in the undeployed configuration having a smaller diameter than in the deployed configuration; and the outer frame is configured to expand from an undeployed configuration to a deployed configuration, the outer frame in the undeployed configuration having a smaller diameter than in the deployed configuration.
Example 156: The prosthetic valve of any example herein, in particular Example 155, wherein the outer frame is configuration to be positioned longitudinally adjacent to the inner frame when the outer frame and the inner frame are both in the undeployed configuration.
Example 157: The prosthetic valve of any example herein, in particular Example 156, wherein the outer frame is configured to be moved from being positioned longitudinally adjacent to the inner frame to be positioned radially outward of the inner frame.
Example 158: The prosthetic valve of any example herein, in particular Examples 155-157, wherein the inner frame includes a proximal end and a distal end, and the outer frame includes a proximal end and a distal end, and the distal end of the outer frame is configured to be positioned adjacent to the proximal end of the inner frame when the outer frame and the inner frame are both in the undeployed configuration.
Example 159: The prosthetic valve of any example herein, in particular Example 158, wherein the outer frame is configured to be assembled to the inner frame with the proximal end of the inner frame positioned adjacent to the proximal end of the outer frame.
Example 160: The prosthetic valve of any example herein, in particular Example 158 or Example 159, wherein the distal end of the outer frame has a greater diameter than the proximal end of the outer frame when the outer frame is in the deployed configuration.
Example 161: The prosthetic valve of any example herein, in particular Examples 151-160, wherein the inner frame includes a plurality of anchors extending distally from the inner frame.
Example 162: The prosthetic valve of any example herein, in particular Example 161, wherein the plurality of anchors is configured to extend over native leaflets of the heart valve.
Example 163: The prosthetic valve of any example herein, in particular Examples 151-162, wherein the outer frame is configured to form a seal with the heart valve.
Example 164: The prosthetic valve of any example herein, in particular Example 163, further comprising a sealing skirt coupled to the outer frame.
Example 165: The prosthetic valve of any example herein, in particular Examples 151-164, wherein the prosthetic valve is configured for implantation within a mitral heart valve or a tricuspid heart valve.
Example 166: A method comprising: deploying a prosthetic valve within a heart valve, the prosthetic valve including: a plurality of prosthetic valve leaflets; an inner frame supporting the plurality of prosthetic valve leaflets; and an outer frame configured to be positioned radially outward of the inner frame and configured to be assembled to the inner frame within a patient's body.
Example 167: The method of any example herein, in particular Example 166, further comprising expanding the inner frame and the outer frame within the patient's body sequentially.
Example 168: The method of any example herein, in particular Example 167, further comprising expanding the inner frame within the patient's body prior to expanding the outer frame within the patient's body.
Example 169: The method of any example herein, in particular Examples 166-168, wherein the inner frame is configured to expand from an undeployed configuration to a deployed configuration, the inner frame in the undeployed configuration having a smaller diameter than in the deployed configuration; and the outer frame is configured to expand from an undeployed configuration to a deployed configuration, the outer frame in the undeployed configuration having a smaller diameter than in the deployed configuration.
Example 170: The method of any example herein, in particular Example 169, further comprising positioning the inner frame longitudinally adjacent to the outer frame on a delivery apparatus when the outer frame and the inner frame are both in the undeployed configuration.
Example 171: The method of any example herein, in particular Examples 166-170, further comprising utilizing one or more tethers extending between the inner frame and the outer frame to draw the outer frame to the inner frame within the patient's body.
Example 172: The method of any example herein, in particular Examples 166-171, further comprising utilizing one or more engagement bodies on one or more of the inner frame or the outer frame to secure the inner frame to the outer frame within the patient's body.
Example 173: The method of any example herein, in particular Examples 166-172, wherein the inner frame includes a plurality of anchors extending distally from the inner frame.
Example 174: The method of any example herein, in particular Examples 166-173, wherein the outer frame is configured to form a seal with the heart valve.
Example 175: The method of any example herein, in particular Examples 166-174, wherein the prosthetic valve is configured for implantation within a mitral heart valve or a tricuspid heart valve.
Example 176: A prosthetic valve for implantation within a heart valve, the prosthetic valve comprising: a plurality of prosthetic valve leaflets; an inner valve body supporting the plurality of prosthetic valve leaflets and configured to be expanded with an inflatable body; and a self-expanding outer valve body configured to extend radially outward from the inner valve body.
Example 177: The prosthetic valve of any example herein, in particular Example 176, wherein the inner valve body includes a frame configured to expand radially outward with the inflatable body.
Example 178: The prosthetic valve of any example herein, in particular Example 177, wherein a length of the frame is configured to reduce and a diameter of the frame is configured to increase upon the frame expanding radially outward with the inflatable body.
Example 179: The prosthetic valve of any example herein, in particular Example 177 or Example 178, wherein the frame has a cylindrical shape.
Example 180: The prosthetic valve of any example herein, in particular Examples 176-179, wherein the inner valve body surrounds a flow channel that the plurality of prosthetic valve leaflets is positioned in, and the flow channel is configured to receive the inflatable body.
Example 181: The prosthetic valve of any example herein, in particular Examples 176-180, wherein the self-expanding outer valve body includes one or more anchors for anchoring to the heart valve.
Example 182: The prosthetic valve of any example herein, in particular Example 181, wherein the one or more anchors are configured to hook over a distal tip of a leaflet of the heart valve.
Example 183: The prosthetic valve of any example herein, in particular Example 181 or Example 182, wherein the one or more anchors are configured to be positioned on an inflow side of the heart valve.
Example 184: The prosthetic valve of any example herein, in particular Examples 181-183, wherein the one or more anchors include at least one distal anchor and at least one proximal anchor.
Example 185: The prosthetic valve of any example herein, in particular Examples 176-184, wherein the self-expanding outer valve body includes a sealing body for sealing with the heart valve.
Example 186: The prosthetic valve of any example herein, in particular Examples 176-185, wherein the self-expanding outer valve body includes a self-expanding frame configured to expand radially outward.
Example 187: The prosthetic valve of any example herein, in particular Example 186, wherein the self-expanding frame is made of a shape memory material.
Example 188: The prosthetic valve of any example herein, in particular Example 186 or Example 187, wherein the inner valve body includes a frame that is less flexible than the self-expanding frame.
Example 189: The prosthetic valve of any example herein, in particular Examples 176-188, wherein the self-expanding outer valve body is configured to deflect to conform to a shape of an annulus of the heart valve.
Example 190: The prosthetic valve of any example herein, in particular Examples 176-189, wherein the prosthetic valve is configured for implantation within a mitral heart valve or a tricuspid heart valve.
Example 191: A method comprising: deploying a prosthetic valve within a heart valve, the prosthetic valve including: a plurality of prosthetic valve leaflets, an inner valve body supporting the plurality of prosthetic valve leaflets and configured to be expanded with an inflatable body, and a self-expanding outer valve body configured to extend radially outward from the inner valve body.
Example 192: The method of any example herein, in particular Example 191, wherein the inner valve body surrounds a flow channel that the plurality of prosthetic valve leaflets is positioned in, and the inflatable body is positioned within the flow channel.
Example 193: The method of any example herein, in particular Example 191 or Example 192, wherein the self-expanding outer valve body includes one or more anchors for anchoring to the heart valve.
Example 194: The method of any example herein, in particular Example 193, wherein the one or more anchors are configured to hook over a distal tip of a leaflet of the heart valve.
Example 195: The method of any example herein, in particular Examples 191-194, wherein the self-expanding outer valve body includes a sealing body for sealing with the heart valve.
Example 196: The method of any example herein, in particular Examples 191-195, wherein the self-expanding outer valve body includes a self-expanding frame configured to expand radially outward.
Example 197: The method of any example herein, in particular Examples 191-196, further comprising allowing the self-expanding outer valve body to deflect to conform to a shape of an annulus of the heart valve.
Example 198: The method of any example herein, in particular Example 197, further comprising allowing the self-expanding outer valve body to deflect to conform to calcification of the heart valve.
Example 199: The method of any example herein, in particular Examples 191-198, further comprising inflating the inflatable body to expand the inner valve body.
Example 200: The method of any example herein, in particular Examples 191-199, wherein the heart valve is a mitral heart valve or a tricuspid heart valve.
Example 201: A system comprising: a prosthetic valve configured to be deployed to a heart valve of a patient's heart, at least a portion of the prosthetic valve having a dimension that has been reduced with electropolishing.
Example 202: The system of any example herein, in particular Example 201, wherein the prosthetic valve includes one or more anchors for anchoring the prosthetic valve to the heart valve, and the dimension of the one or more anchors has been reduced with the electropolishing.
Example 203: The system of any example herein, in particular Example 202, wherein each of the one or more anchors has a circumferential width and a radial thickness, and the radial thickness of the one or more anchors has been reduced with the electropolishing.
Example 204: The system of any example herein, in particular Example 203, wherein the radial thickness of the one or more anchors is tapered with the electropolishing.
Example 205: The system of any example herein, in particular Example 203 or Example 204, wherein the one or more anchors has a tip portion, a first intermediate portion positioned adjacent to the tip portion and a second intermediate portion positioned such that the first intermediate portion is between the tip portion and the second intermediate portion, and the second intermediate portion has a lesser radial thickness than the first intermediate portion.
Example 206: The system of any example herein, in particular Examples 202-205, wherein the dimension of the one or more anchors having been reduced increases a flexibility of the one or more anchors.
Example 207: The system of any example herein, in particular Examples 202-206, wherein the prosthetic valve includes a valve frame having a distal portion, and each of the one or more anchors is configured to extend from the distal portion of the valve frame in a proximal direction.
Example 208: The system of any example herein, in particular Example 207, wherein each of the one or more anchors is configured to extend over a tip of a native heart valve leaflet.
Example 209: The system of any example herein, in particular Examples 201-208, wherein the prosthetic valve includes a plurality of prosthetic valve leaflets and a plurality of struts coupled to the plurality of prosthetic valve leaflets, at least one of the plurality of struts having the dimension that has been reduced with the electropolishing.
Example 210: The system of any example herein, in particular Examples 201-209, wherein the prosthetic valve is configured to be deployed to a mitral heart valve or a tricuspid heart valve.
Example 211: A method comprising: electropolishing at least a portion of a body for use with a prosthetic valve, the electropolishing reducing a dimension of at least the portion of the body.
Example 212: The method of any example herein, in particular Example 211, wherein the electropolishing reduces the dimension of one or anchors for the prosthetic valve.
Example 213: The method of any example herein, in particular Example 212, wherein the dimension comprises a radial thickness of the one or more anchors.
Example 214: The method of any example herein, in particular Example 213, wherein the radial thickness of the one or more anchors is tapered with the electropolishing.
Example 215: The method of any example herein, in particular Example 213 or Example 214, wherein the one or more anchors has a tip portion, a first intermediate portion positioned adjacent to the tip portion and a second intermediate portion positioned such that the first intermediate portion is between the tip portion and the second intermediate portion, and the second intermediate portion has a lesser radial thickness than the first intermediate portion.
Example 216: The method of any example herein, in particular Examples 211-215, wherein the body comprises one or more anchors for the prosthetic valve.
Example 217: The method of any example herein, in particular Examples 211-216, wherein the body comprises a cylinder having been cut to include one or more anchors for the prosthetic valve.
Example 218: The method of any example herein, in particular Examples 211-217, wherein the body comprises a cylinder, and the method further comprises: electropolishing at least the portion of the cylinder to reduce the dimension of at least the portion of the cylinder; and cutting the cylinder to include one or more anchors for the prosthetic valve following the electropolishing.
Example 219: The method of any example herein, in particular Examples 211-218, wherein the portion of the body comprises a first portion, and the method further comprises covering a second portion of the body to reduce the electropolishing of the second portion of the body while the first portion is being electropolished.
Example 220: The method of any example herein, in particular Examples 211-219, further comprising producing a prosthetic valve utilizing the body.
Example 221: A system comprising: a prosthetic valve configured to be deployed to a heart valve of a patient's heart, at least a portion of the prosthetic valve being configured to include one or more drugs for interaction with the patient's body.
Example 222: The system of any example herein, in particular Example 221, wherein the prosthetic valve includes a frame, and at least a portion of the frame includes the one or more drugs.
Example 223: The system of any example herein, in particular Example 222, wherein the one or more drugs are coated on the frame.
Example 224: The system of any example herein, in particular Example 222 or Example 223, wherein the frame includes one or more recesses for receiving the one or more drugs.
Example 225: The system of any example herein, in particular Examples 221-224, wherein the prosthetic valve includes a skirt, and at least a portion of the skirt includes the one or more drugs.
Example 226: The system of any example herein, in particular Example 225, wherein the skirt comprises a sealing skirt for forming a seal with at least a portion of the heart valve of the patient's heart.
Example 227: The system of any example herein, in particular Examples 221-226, wherein the prosthetic valve includes one or more anchors for anchoring the prosthetic valve to the heart valve, and at least a portion of the one or more anchors includes the one or more drugs.
Example 228: The system of any example herein, in particular Examples 221-227, wherein the prosthetic valve includes one or more sutures, and at least a portion of the one or more sutures includes the one or more drugs.
Example 229: The system of any example herein, in particular Examples 221-228, wherein the prosthetic valve includes a plurality of prosthetic valve leaflets, and at least a portion of one of the prosthetic valve leaflets includes the one or more drugs.
Example 230: The system of any example herein, in particular Examples 221-229, wherein the prosthetic valve includes an inner valve body and an outer valve body and a pocket between the inner valve body and the outer valve body, the pocket configured to include the one or more drugs.
Example 231: The system of any example herein, in particular Example 230, wherein the pocket is configured to be filled with the one or more drugs in vivo.
Example 232: The system of any example herein, in particular Example 230 or Example 231, wherein the outer valve body comprises a sealing body for forming a seal with at least a portion of the heart valve of the patient's heart, and the inner valve body supports a plurality of prosthetic valve leaflets.
Example 233: The system of any example herein, in particular Examples 221-232, wherein the one or more drugs include an antithrombosis drug.
Example 234: The system of any example herein, in particular Examples 221-233, wherein the one or more drugs are configured to encourage tissue ingrowth with the prosthetic valve.
Example 235: The system of any example herein, in particular Examples 221-234, wherein the one or more drugs are bioresorbable.

Any of the features of any of the examples, including but not limited to any of the first through 235 examples referred to above, is applicable to all other aspects and examples identified herein, including but not limited to any examples of any of the first through 235 examples referred to above. Moreover, any of the features of an example of the various examples, including but not limited to any examples of any of the first through 235 examples referred to above, is independently combinable, partly or wholly with other examples described herein in any way, e.g., one, two, or three or more examples may be combinable in whole or in part. Further, any of the features of the various examples, including but not limited to any examples of any of the first through 235 examples referred to above, may be made optional to other examples. Any example of a method can be performed by a system or apparatus of another example, and any aspect or example of a system or apparatus can be configured to perform a method of another aspect or example, including but not limited to any examples of any of the first through 235 examples referred to above.

In closing, it is to be understood that although aspects of the present specification arc highlighted by referring to specific examples, one skilled in the art will readily appreciate that these disclosed examples are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular examples only and is not intended to limit the scope of systems, apparatuses, and methods as disclosed herein, which is defined solely by the claims. Accordingly, the systems, apparatuses, and methods are not limited to that precisely as shown and described.

Certain examples of systems, apparatuses, and methods are described herein, including the best mode known to the inventors for carrying out the same. Of course, variations on these described examples will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described examples in all possible variations thereof is encompassed by the systems, apparatuses, and methods unless otherwise indicated herein or otherwise clearly contradicted by context.

Groupings of alternative examples, elements, or steps of the systems, apparatuses, and methods are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses an approximation that may vary, yet is capable of performing the desired operation or process discussed herein.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A system comprising:
a prosthetic valve including:
a plurality of prosthetic valve leaflets;
an inner frame for supporting the plurality of prosthetic valve leaflets, the inner frame having a proximal end and a distal end; and
an anchoring body positioned radially outwardly of the inner frame for anchoring to a native heart valve, the anchoring body including a proximal frame portion and a distal frame portion, wherein an annular recess is provided between the proximal frame portion and the distal frame portion for receiving an annulus of a native heart valve;
wherein a proximal end of the proximal frame portion is positioned proximal to the proximal end of the inner frame, and
wherein a distal end of the distal frame portion is positioned distal to the distal end of the inner frame.

2. The system of claim 1, wherein the anchoring body is self-expanding.

3. The system of claim 2, wherein the inner frame is balloon expandable.

4. The system of claim 1, wherein the inner frame has a substantially cylindrical shape and wherein the prosthetic valve leaflets are positioned within a lumen of the inner frame.

5. The system of claim 1, wherein the proximal end of the proximal frame portion is positioned radially outwardly from the proximal end of the inner frame.

6. The system of claim 5, wherein the distal end of the distal frame portion is positioned radially outwardly from the distal end of the inner frame.

7. The system of claim 1, wherein the proximal frame portion and the distal frame portion are independently deployable.

8. The system of claim 1, wherein the anchoring body includes at least one strut having an undulating shape for enhanced flexibility.

9. The system of claim 1, wherein the proximal frame portion extends radially outward to form a first shoulder positioned proximal of the annular recess, and the distal frame portion extends radially outward to form a second shoulder positioned distal of the annular recess and wherein the first and second shoulders are shaped for receiving the annulus of the native heart valve therebetween.

10. The system of claim 1, wherein the prosthetic valve is for replacing a mitral valve or a tricuspid valve.

11. A prosthetic valve for implantation within a native heart valve, the prosthetic valve comprising:
a plurality of prosthetic valve leaflets;
an inner valve body supporting the plurality of prosthetic valve leaflets and configured to be expanded with an inflatable body; and
a self-expanding outer valve body configured to extend radially outward from the inner valve body.

12. The prosthetic valve of claim 11, wherein the inner valve body includes a frame adapted to expand radially outward with the inflatable body, and the self-expanding outer valve body includes a self-expanding frame adapted to expand radially outward.

13. The prosthetic valve of claim 11, wherein the self-expanding outer valve body includes one or more anchors for anchoring to the native heart valve.

14. The prosthetic valve of claim 13, wherein the one or more anchors are configured to hook over a distal tip of a leaflet of the native heart valve.

15. The prosthetic valve of claim 1, wherein the prosthetic valve is for replacing a mitral valve or a tricuspid valve.

16. A prosthetic valve, comprising:
a plurality of prosthetic valve leaflets;
an inner frame for supporting the plurality of prosthetic valve leaflets; and
an anchoring body positioned radially outward of the inner frame and shaped for anchoring to a native heart valve, the anchoring body including:
a proximal frame portion and a distal frame portion, wherein an annular recess is provided between the proximal frame portion and the distal frame portion for receiving tissue of a native heart valve, the proximal frame portion forming a first shoulder positioned proximal of the annular recess, and the distal frame portion forming a second shoulder positioned distal of the annular recess, and wherein the proximal frame portion includes a proximal end spaced from the inner frame, and the distal frame portion includes a distal end spaced from the inner frame.

17. The prosthetic valve of claim 16, further comprising one or more tethers coupling the anchoring body to the inner frame.

18. The prosthetic valve of claim 16, wherein the anchoring body is self-expanding, and the inner frame is configured to be expanded using an inflatable body.

19. The prosthetic valve of claim 16, wherein at least a portion of the prosthetic valve has a dimension that has been reduced with electropolishing.

20. The prosthetic valve of claim 16, wherein at least a portion of the prosthetic valve is coated with a drug.
